SEQUENCE LISTING (8 sequences)

SEQ ID NO: 1 - protein, 331 aa, human
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLTSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 2 - protein, 217 aa, human
LPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDLKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 3 - protein, 217 aa, human
LPDSVDWREKGCVTEVKYQGSCGASWAFSAVGALEAQLKLKTGKLVSLSAQNLVDCSTEKYGNKGCNGGFMTTAFQYIIDNKGIDSDASYPYKAMDLKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 4 - protein, 217 aa, human
LPDSVDWREKGCVTEVKYQGSCGASWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 5 - protein, 217 aa, human
LPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 6 - protein, 331 aa, human
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLTSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDLKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 7 - protein, 331 aa, human
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLTSSLRVPSQWQRNITYKSNPNRILPDSVDWREKGCVTEVKYQGSCGASWAFSAVGALEAQLKLKTGKLVSLSAQNLVDCSTEKYGNKGCNGGFMTTAFQYIIDNKGIDSDASYPYKAMDLKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 8 - protein, 331 aa, human
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLTSSLRVPSQWQRNITYKSNPNRILPDSVDWREKGCVTEVKYQGSCGASWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

(19) United States
(12) Patent Application Publication
(10) Pub. No.: US 2003/0143714 A1
(43) Pub. Date: Jul. 31, 2003

(54) Crystal structure of a mutant of cathepsin S enzyme

(75) Inventors: Marieke B.A.C. Lamers, Cambridge (GB); David H. Williams, Cambridge (GB); Johan P. Turkenburg, York (GB); Roderick E. Hubbard, York (GB)

Correspondence Address: BIRCH STEWART KOLASCH & BIRCH, PO BOX 747, FALLS CHURCH, VA, 22040-0747, US

(73) Assignee: Medivir UK Ltd., Cambridge (GB)

(21) Appl. No.: 10/273,577

(22) Filed: Oct. 18, 2002

Related U.S. Application Data
(60) Provisional application No. 60/330,191, filed on Oct. 19, 2001.

(51) Int. Cl. G06F 19/00; G01N 33/48; G01N 33/50; C12N 9/64

(52) U.S. Cl. 435/226; 702/19

(57) ABSTRACT

The invention relates to the X-ray crystal structure of a cathepsin S mutant. The invention further relates to an apparatus programmed with one or more of the structure coordinates of the cathepsin S binding pockets, wherein said apparatus is capable of displaying a three-dimensional representation of that binding pocket.

### TECHNICAL FIELD OF INVENTION

The present invention relates to crystals of a mutant cathepsin S (catS) enzyme and more particularly to the high resolution structure of a mutant catS obtained by X-ray diffraction. This invention also relates to other mutants of catS. In addition, this invention relates to methods of using the structure coordinates of a mutant catS and other mutant catS to screen and design compounds that bind to the active site and accessory binding site of catS.

### BACKGROUND ART

Cathepsin S is a single-chain cysteinyl proteinase of the papain superfamily, highly stable at neutral or slightly acidic pH, which was first isolated from bovine lymph nodes and spleen (Kirschke et al., 1986; Turnsek et al., 1975). Subsequent investigations have shown that expression of cathepsin S is almost exclusively restricted to cells of lymphoid origin (Kirschke et al., 1989; Qian et al., 1991; Shi et al., 1994). Interest has recently focused on cathepsin S and its role in immune system regulation.

Extracellular protein antigens are transported into antigen presenting cells via endocytosis or phagocytosis. These protein antigens must be digested to small peptides which are then loaded onto the binding groove of major histocompatibility complex (MHC) class II and presented for recognition by CD4+ T lymphocytes (Germain & Margulies, 1993). Invariant chain (Ii) is removed from αβ MHC class II dimers by gradual regulated cleavage of Ii by lysosomal proteinases (Cresswell, 1996; Newcomb & Cresswell, 1993; Roche & Cresswell, 1991) including cathepsin S (Riese et al., 1998; Riese et al., 1996). Indeed, mouse gene knockout experiments have demonstrated that cathepsin S deficiency results in a block in the processing of MHC class II-associated invariant chain Ii leading to markedly delayed MHC class II peptide loading in B lymphocytes and dendritic cells (Nakagawa et al., 1999; Shi et al., 1999). Moreover, administration of the selective irreversible cathepsin S inhibitor LHVS to mice results in accumulation of a Ii breakdown product, attenuation of MHC class II peptide complex formation and inhibition of antigen presentation (Riese et al., 1998). For this reason, cathepsin S is considered a target for autoimmune disease therapy. However, inhibition of the closely related family members cathepsin L and K could lead to changes in skin and hair and bone remodeling (Gowen et al., 1999; Hofbauer & Heufelder, 1999; Nakagawa et al., 1998; Saftig et al., 1998), highlighting the need for inhibitor selectivity.

A homology model for cathepsin S was published in 1997 (Sumpter et al., 1997), but provides little guidance for the modeling of inhibitors which are potent against cathepsin S without also inhibiting other members of the papain superfamily. A 2.5 Å crystal structure of cathepsin S liganded to and distorted by a potent irreversible vinyl sulfone inhibitor, APC 2848 has been published (McGrath et al., 1998). As is apparent from McGrath, however, this structure was based on a single crystal of questionable quality and a significant amount of the structure has been inferred from homologies with cathepsin K. Accordingly this publication is not suitable for accurate modeling of selective cathepsin S inhibitors. It will thus be apparent that the prior art has been unable to prepare cathepsin S crystals unliganded or liganded with reversible inhibitors which would allow the detailed structure of the active site to be elucidated. Thus, x-ray crystallographic analysis of such proteins has not been possible, thereby hampering development of effective drugs.

### SUMMARY OF THE INVENTION

The present invention solves this problem by providing, for the first time, a crystalizable mutant of the catS enzyme.

It is an object of the invention to provide a crystalline catS polypeptide, or a variant thereof, free of any irreversible inhibitor bound thereto, for solving the three-dimensional structure of the catS enzyme and to determine its structure coordinates.

It is an object of the invention to provide a crystalizable composition comprising a Cathepsin S polypeptide, free of any irreversible inhibitor bound thereto, for solving the three-dimensional structure of the catS enzyme and to determine its structure coordinates.

It is a further object of the invention to provide catS mutants characterized by one or more different properties as compared with wild-type catS. These properties include altered surface charge, altered substrate specificity or altered specific activity, including tendency to autodigestion. catS mutants are useful for producing high concentrations of cathepsin S for crystallography and other assays and for identifying those amino acids that are most important for the enzymatic activity of catS. This information, in turn, allows the design of catS inhibitors.

It is also an object of the invention to provide a method that uses the structure coordinates and atomic details of catS, or its mutants or homologues or co-complexes, to design, evaluate computationally, synthesize and use inhibitors of catS that avoid the undesirable physical and pharmacologic properties of the current proteinase inhibitors.

It is also an object of the invention to provide a computer for producing a three-dimensional representation of a molecule or molecular complex, which molecule or molecular complex comprises at least one of the binding pockets of cathepsin S. The computer comprises a computer-readable data storage material encoded with computer-readable data (which comprises the structure coordinates of cathepsin S binding pocket amino acids), a working memory for storing instructions for processing the computer-readable data, a central-processing unit coupled to the working memory and to the computer-readable data storage medium for processing the computer-machine readable data into said three-dimensional representation and a display coupled to the central-processing unit for displaying said three-dimensional representation.

It is also an object of the invention to provide a computer for determining at least a portion of the structure coordinates corresponding to X-ray diffraction data obtained from a molecule of molecular complex of cathepsin S. The Computer comprises a computer-readable data storage medium, a computer-readable data storage medium, a working memory for storing instructions for processing the computer-readable data, a central processing unit coupled to the working memory and to the computer-readable data storage medium for performing a Fourier transform of the machine readable data and for processing the computer-readable data into structure coordinates and a display coupled to the central-processing unit for displaying the structure coordinates of the cathepsin S molecule or molecular complex.

It is a also object of the invention to provide a method for evaluating the potential of a chemical entity to associate with a cathepsin S molecule, molecular complex, homologue or homologue complex that comprises the steps of employing computational means to perform a fitting operation between the chemical entity and at least one cathepsin S binding pocket and analyzing the results to quantify the association between the chemical entity and the binding pocket.

It is also an object of the invention to provide a method for producing an inhibitor of a molecule comprising a cathepsin S-like binding pocket comprising the steps of using the atomic coordinates of the cathepsin S binding pockets to generate a three-dimensional structure of the molecule comprising a cathepsin S-like binding pocket, using the three-dimensional structure to design or select the potential agonist or antagonist and synthesizing the agonist or antagonist.

The invention allows the modeling and provision of cathepsin S inhibitors which do not substantially inhibit other members of the papain superfamily.

The use of the invention will thus lead to methods for the treatment of autoimmune disease by administration to a patient in need thereof an effective amount of an inhibitor of cathepsin S.

It is also an object of the invention to provide a method for identifying a potential inhibitor of a molecule comprising a cathepsin S-like binding pocket comprising the steps of using the atomic coordinates of the cathepsin S binding pockets to generate a three-dimensional structure of the molecule comprising a cathepsin S-like binding pocket, using the three-dimensional structure to design or select the potential inhibitor and synthesizing the inhibitor and contacting the inhibitor with an active cathepsin S enzyme or fragment thereof to determine the ability of the inhibitor to interact with cathepsin S.

### BRIEF DESCRIPTION OF THE DRAWINGS

FIG. 1 represents the Cα trace of papain (9PAP.pdb-purple) and cathepsin L (1CS8.pdb-green), K (1MEM.pdb-red), H (8PCH.pdb-yellow) and S (blue).

FIG. 2 stereo picture of Cα-Cα trace of cathepsin S. The figure was produced using Sybyl 6.6 (Tripos).

FIG. 3 represents a ribbon model of cathepsin S with the catalytic triad Cys25→Ser, His164 and Asn184 shown as ball and sticks color coded by atom type.

FIG. 4 represents the hydrogen bonding network in the active site. The catalytic triad (with the Ser25 mutation) is shown on the right, with three water molecules in the center and Gln19 and Trp186 to the left. Hydrogen bonds are shown as dashed lines. The observed network may provide a mechanism for correct catalytic residue side chain orientation prior to substrate hydrolysis.

FIG. 5 represents the Final Maximum Likelihood-weighted electron density map (2Fₒ-F_{c}) contoured at 1σ above the mean. The catalytic residues for cathepsin K and mutant cathepsin S are represented by ball and stick models with atom-specific colors: oxygen-red, nitrogen-blue, carbon-gray. This figure shows that the constellation of the active site residues is essentially unchanged in the mutant.

FIG. 6 represents a view of the substrate-binding cleft of cathepsin S showing the proposed substrate-binding sites S3 to S1′. The enzyme is shown with a surface colored by electrostatic potential (Gasteiger charges).

FIG. 7 is a 2D representation of the residues present in the non-prime side of the cathepsin S substrate binding site. For substrate specificity comparisons, the equivalent residues in cathepsin K (1MEM.pdb) and cathepsin L (1CS8.pdb) are shown in blue and red, respectively.

FIG. 8 is a block diagram of an exemplary computer system for implementing 3-dimensional modeling of a molecule or molecular complex according to principles of the present invention.

FIG. 9 shows a cross section of a magnetic storage medium.

FIG. 10 shows a cross section of an optically-readable data storage medium.

TABLE 1 lists the data collection and refinement statistics, and the model statistics.

TABLE 2 lists the crystallographic coordinate transformation data.

TABLE 3 lists the atomic structure coordinates for catS as derived by X-ray diffraction from a crystal of the mutant catS. The following abbreviations are used in TABLE 3:

“#” refers to the atom serial number.

“Atom type” refers to the element whose coordinates are measured. The first letter in the column defines the element.

“Res” denotes the amino acid residues' name using the three letter code. The abbreviations used are listed below.

“Chn I.D.” is the chain identifier.

“Res #” provides the residue sequence number.

“X coord.,” “Y coord.” And “Z coord.” represent the values for the S, Y and Z coordinates and crystallographic ally define the atomic position of the element measured.

“Occ” is an occupancy factor that refers to the fraction of the molecules in which each atom occupies the position specified by the coordinates. A value of “1” indicates that each atom has the same conformation, i.e., the same position, in all molecules of the crystal.

“Temp fact” is a thermal factor that measures movement of the atom around its atomic center.

“Atomic #” provides the correct atomic number.

Structure coordinates for catS according to TABLE 3 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the above.

Abbreviations and Definitions

Abbreviations

Amino Acids

A=Ala=Alanine

V=Val=Valine

L=Leu=Leucine

I=Ile=Isoleucine

P=Pro=Proline

F=Phe=Phenylalanine

W=Trp=Tryptophan

M=Met=Methionine

G=Gly=Glycine

S=Ser=Serine

T=Thr=Threonine

C=Cys=Cysteine

Y=Tyr=Tyrosine

N=Asn=Asparagine

Q=Gln=Glutamine

D=Asp=Aspartic Acid

E=Glu=Glutamic Acid

K=Lys=Lysine

R=Arg=Arginine

H=His=Histidine

Definitions

The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine,, arginine, and lysine. Unless specifically indicated, all amino acids referred to in this application are in the L-form.

The term “unnatural amino acids” means amino acids that are not naturally found in proteins. Examples of unnatural amino acids include racemic mixtures of selenocysteine and selenomethionine. In addition, unnatural amino acids include the D or L forms of nor-leucine, γ-carboxyglutamic acid, ornithine, para-nitrophenylalanine, homophenylalanine, para-fluorophenylalanine, 3-amino-2-benzylpropionic acid, homoarginine, and D-phenylalanine.

The term “positively charged amino acid” includes any naturally occurring or unnatural amino acid having a positively charged side chain under normal physiological conditions. Examples of positively charged naturally occurring amino acids are arginine, lysine and histidine, with ornithine representing a non-natural amino acid

The term “negatively charged amino acid” includes any naturally occurring or unnatural amino acid having a negatively charged side chain under normal physiological conditions. Examples of negatively charged naturally occurring amino acids are aspartic acid and glutamic acid.

The term “hydrophobic amino acid” means any amino acid having an uncharged, nonpolar side chain that is relatively insoluble in water. Examples of naturally occurring hydrophobic amino acids are alanine, leucine, isoleucine, valine, proline, phenylalanine, tryptophan and methionine.

The term “hydrophilic amino acid” means any amino acid having an uncharged, polar side chain that is relatively soluble in water. Examples of naturally occurring hydrophilic amino acids are serine, threonine, tyrosine, asparagine, glutamine, and cysteine.

The term “mutant” refers to a nucleic acid that encodes a catS polypeptide, but whose sequence differs from the wild-type catS nucleic acid. Such a mutant may be prepared, for example, by expression of catS cDNA previously altered in its coding sequence by oligonucleotide-directed mutagenesis. The mutant would preferably contain 80%, 85%, 90% or 95% sequence identity with the wild-type catS nucleic acid. More preferably, the mutant would contain 96%, 97%, 98%, 99% or 99.5% identity with the wild-type catS nucleic acid sequence.

The term “variant” refers to a catS polypeptide, i.e characterized by the replacement of at least one amino acid from the wild-type, human catS sequence according to Shi et al. (1994) J Biol Chem 269:11530-6 (SEQ ID NO: 1). Such a variant may be prepared, for example, by expression of catS cDNA previously altered in its coding sequence by oligonucleotide-directed mutagenesis. This polypeptide may or may not display the biological activity of wild-type, human catS, but would contain all or part of the catS active site. The variant would not possess substantial amounts of the catalytic activity of cathepsin B, H, K or L. A variant containing substituted amino acids retains the overall spatial juxtaposition of the binding pockets and their associated key functional residues.

catS mutants may also be generated by site-specific incorporation of unnatural amino acids into catS proteins using the general biosynthetic method of Noren et al. (1989) Science, 244: 182-188. In this method, the codon encoding the amino acid of interest in wild-type catS is replaced by a “blank” nonsense codon, TAG, using oligonucleotide-directed mutagenesis (described in detail, infra). A suppressor tRNA directed against this codon is then chemically aminoacylated in vitro with the desired unnatural amino acid. The aminoacylated tRNA is then added to an in vitro translation system to yield a mutant catS enzyme with the site-specific incorporated unnatural amino acid.

Selenocysteine or selenomethionine may be incorporated into wild-type or mutant catS by expression of catS-encoding cDNAs in auxotrophic E. coli strains (Hendrickson et al. (1990) EMBO J. 9(5): 1665-1672). In this method, the wild-type or mutagenized catS cDNA may be expressed in a host organism on a growth medium depleted of either natural cysteine or methionine (or both) but enriched in selenocysteine or selenomethionine (or both).

The term “altered surface charge” means a change in one or more of the charge units of a mutant polypeptide, at physiological pH, as compared to wild-type catS. This is preferably achieved by mutation of at least one amino acid of wild-type catS to an amino acid comprising a side chain with a different charge at physiological pH than the original wild-type side chain.

The change in surface charge is determined by measuring the isoelectric point (pI) of the polypeptide molecule containing the substituted amino acid and comparing it to the isoelectric point of the wild-type catS molecule. The pI of wild-type human catS is between 8.3 and 8.6 (Bromme et al. (1993) J Biol Chem 268:4832-8).

The term “altered substrate specificity” refers to a change in the ability of a mutant catS to cleave a substrate as compared to wild-type catS. Substrate specificity may be measured using the method described by Bromme et al. (1993) J Biol Chem 268:4832-8.

The “kinetic form” of catS refers to the condition of the enzyme in its free or unbound form or bound to a chemical entity at either its active site or accessory binding site.

A “competitive” inhibitor is one that inhibits catS activity by binding to the same kinetic form of catS as its substrate binds, thus directly competing with the substrate for the active site of catS. Competitive inhibition can be reversed completely by increasing the substrate concentration.

An “uncompetitive” inhibitor is one that inhibits catS by binding to a different kinetic form of the enzyme than does the substrate. Such inhibitors bind to catS already bound with the substrate and not to the free enzyme. Uncompetitive inhibition cannot be reversed completely by increasing the substrate concentration.

A “non-competitive” inhibitor is one that can bind to either the free or substrate bound form of catS.

Those of skill in the art may identify inhibitors as competitive, uncompetitive or non-competitive, by computer fitting enzyme kinetic data using standard equations according to Segel, Enzyme Kinetics, J. Wiley & Sons, (1975). It should also be understood that uncompetitive or non-competitive inhibitors according to this invention may bind to the accessory binding site.

The term “homologue” means a protein having at least 30% amino acid sequence identity with catS or any functional domain of catS.

The term “co-complex” means catS or a mutant or homologue of catS in covalent or non-covalent association with a chemical entity or compound.

The term “associating with” refers to a condition of proximity between a chemical entity or compound, or portions thereof, and a catS molecule or portions thereof. The association may be non-covalent, wherein the juxtaposition is energetically favored by hydrogen bonding or van der Waals or electrostatic interactions or it may be covalent.

The term “β-sheet” refers to the conformation of a polypeptide chain stretched into an extended zig-zig conformation. Portions of polypeptide chains that run “parallel” all run in the same direction. Polypeptide chains that are “antiparallel” run in the opposite direction from the parallel chains. A barrel refers to a sheet which has substantially or completely curled to define an internal volume.

The term “α-helix” refers to a helical, or spiral, configuration of a polypeptide chain in which successive turns of the helix are held together by hydrogen bonds between the amide (peptide) links, the carbonyl group of any given residue being hydrogen-bonded to the imino group of the third residue behind it in the chain.

The term “catalytic triad” refers to the residues contributing to the catalytic mechanism of papain-like enzymes, typically Cys25, Asn184 and His168.

The term “active site” or “active site moiety” refers to any or all of the following sites in catS: the substrate binding site, the catalytic triad and the site where the cleavage of a substrate occurs. The active site is typically characterized by at least amino acid residues 19, 23-26, 62-64, 67-71, 137, 138, 162, 163-165 and 211 using the sequence of the 217 amino acid mature protein (SEQ ID NOs: 2-5).

The term “binding pocket” refers to a binding subsite, or portion of the binding site on the catS molecule.

The “S₁ binding pocket” of the catS active site is defined as the space typically surrounded by amino acid residue Gln19, Gly23 and Cys25. Asn 163 verges on the S₁′, S₁ and S₂ binding pockets.

The “S₂ binding pocket” of the catS active site is typically defined as the space surrounded by amino acid residues Met71, Gly137, Val138, Val162,, Gly165 and Phe211. Asn 163 verges on the S₁′, S₁ and S₂ binding pockets.

The “S₃ binding pocket” of the catS active site is typically defined as the space surrounded by amino acid residues Gly62, Asn63, Lys64, Asn67, Gly68 and Gly69.

The “S₁′ binding pocket” of the catS active site is typically defined as the space surrounded by amino acid residues Ala140, Phe 145 and Trp186. Asn 163 verges on the S₁′, S₁ and S₂ binding pockets.

The term “structure coordinates” refers to mathematical coordinates derived from mathematical equations related to the patterns obtained on diffraction of a monochromatic beam of X-rays by the atoms (scattering centers) of a catS molecule in crystal form. The diffraction data are used to calculate an electron density map of the repeating unit of the crystal. The electron density maps are used to establish the positions of the individual atoms within the unit cell of the crystal.

The term “heavy atom derivatization” refers to the method of producing a chemically modified form of a crystal of catS. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the enzyme. Blundel, T. L. and N. L. Johnson, Protein Crystallography, Academic Press (1976).

Those of skill in the art understand that a set of structure coordinates determined by X-ray crystallography is not without standard error. For the purpose of this invention, any set of structure coordinates for catS or catS homologues or catS mutants that have a root mean square deviation of protein backbone atoms (N, Cα, C and 0) of less than 1.5 Å when superimposed, using backbone atoms, on the structure coordinates listed in TABLE 3 shall be considered identical.

The term “unit cell” refers to a basic parallelipiped shaped block. The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal.

The term “molecular replacement” refers to a method that involves generating a-preliminary model of an catS crystal whose structure coordinates are unknown, by orienting and positioning a molecule whose structure coordinates are known (e.g., catS coordinates from TABLE 3) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, In Methods in Enzymology, 115, pp. 55-77 (1985); M. G. Rossmann, ed., “The Molecular Replacement Method”, Int. Sci. Rev. Ser., No. 13, Gordon & Breach, New York, (1972). Using the structure coordinates of catS provided by this invention, molecular replacement may be used to determine the structure coordinates of a crystalline mutant or homologue of catS or of a different crystal form of catS.

The term “peptidomimetic inhibitors” refers to an inhibitor, typically a compound resembling the peptide substrate of the catS enzyme, but derivatized by replacement of side chains, C-terminus, N-terminus or peptide bonds, to enhance binding within the active site.

### DETAILED DESCRIPTION OF THE INVENTION

The present invention relates to crystalline cathepsin S enzyme (catS), the structure of catS as determined by X-ray crystallography, the use of that structure to solve the structure of catS homologues and of other crystal forms of catS, mutants and co-complexes of catS and the use of the catS structure and that of its homologues, mutants and co-complexes to design inhibitors of catS.

A. The Structure of catS

The present invention provides, for the first time, crystals of a human catS mutant as well as the structure of catS as determined therefrom. The sequence of the mature wild-type catS enzyme (SEQ ID NO: 5) was altered by replacing Cys25 with Ser to generate the mutant (SEQ ID NO: 4). The crystals generated from the mutant have a rod shape and belong to the trigonal space group P3₁21 with a=b=80.0 Å, c=61.5 Å. Assuming one protein molecule in the asymmetric unit, the Matthews coefficient (Matthews (1968) J. Mol. Biol. 33(2):491-7) is 2.3 Å³/Da, corresponding to a solvent content of 46%. Data statistics are given in TABLE 1.

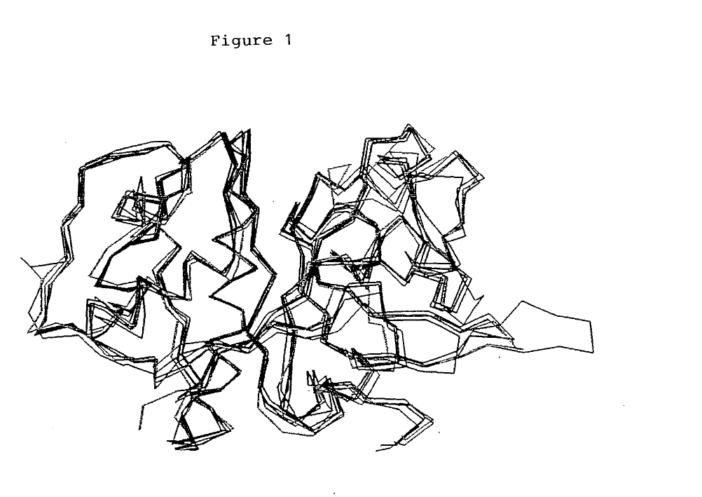

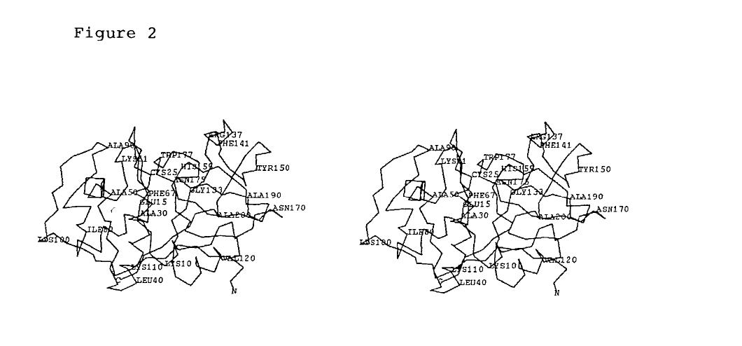

There is one protein molecule per asymmetric unit. The structure of catS is very similar to that of the plant protein papain and the Cα trace for the mutant and those for the papain superfamily are likewise similar (see FIG. 1). A stereo picture of Cα-Cα trace of cathepsin S appears in FIG. 2.

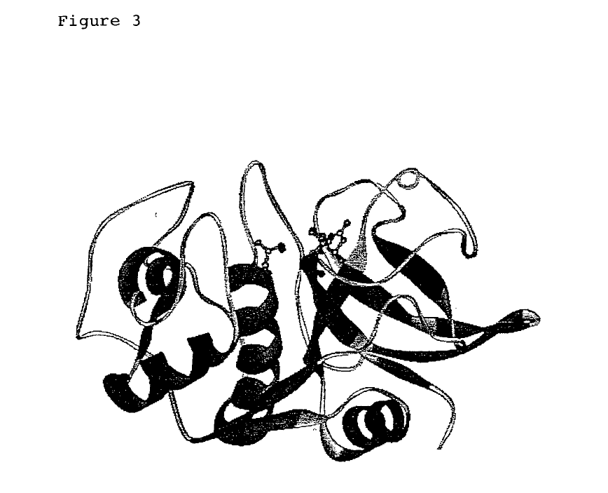

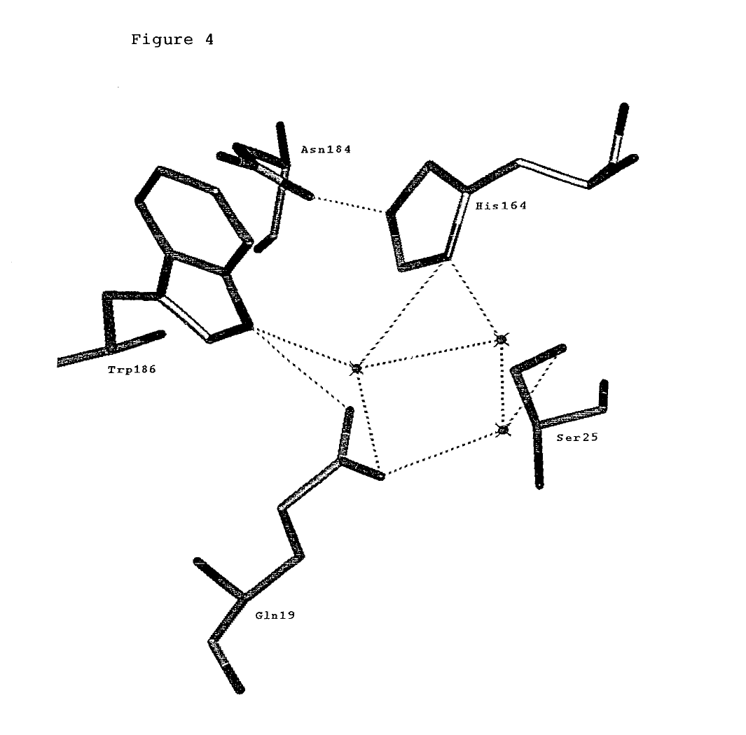

Cathepsin S forms a monomeric structure consisting of two domains. The left domain contains three helices and a hydrophobic core, whereas the right domain consists of a series of antiparallel β-sheets and two α-helices (FIG. 3). Three disulphides, two in the left and one in the right domain, play a role in maintaining the overall structure of the protein. The relative position of the two domains is stabilized by numerous hydrogen bonds between the mainly polar residues lining the two walls of the cleft. In addition, the N-terminus from the left domain crosses over to the right domain and the C-terminus of the right domain in turn crosses over to the left domain, thereby anchoring the two domains. The interface between the two domains forms a deep cleft containing the catalytic triad. The catalytic histidine 164 and the stabilizing asparagine 184 residue of the triad, are part of the polar surface formed by the wall of the right domain, with the left domain contributing the mutated serine 25 at the N-terminus of the main helix in this domain. In the active site a number of well-defined water molecules were identified, which are involved in an intricate hydrogen bonding network (FIG. 4). The mutated Ser 25 residue hydrogen bonds to the backbone amide nitrogen of the catalytic histidine residue and to a water molecule which in turn forms hydrogen bonds to the side chain nitrogen of Gln19 and a second water molecule. This second water molecule hydrogen bonds to one of the side chain nitrogens of the catalytic histidine and to a third water molecule forming hydrogen bonds to the second water and the side chain of Trp186, which closes the network back to the side chain oxygen of Gln19. It is this Gln19 which forms part of the oxyanion hole, a structural feature believed to stabilize the tetrahedral intermediate in the reaction pathway. The hydrogen bond between the catalytic histidine and the Asn 184 is 2.7 Å, in accordance with the distance observed in other catalytic triads. In a number of uncomplexed structures of serine proteases this hydrogen bond was thought not to be present (Matthews et al. (1977) J Biol Chem 252(24): 8875-83). More recent evidence suggests there may be a weak hydrogen bond (Tsukada & Blow (1985) J Mol Biol 184(4): 703-11) as present in this mutant structure. The observed network may provide a mechanism for correct orientation of the catalytic residue side chains prior to substrate hydrolysis.

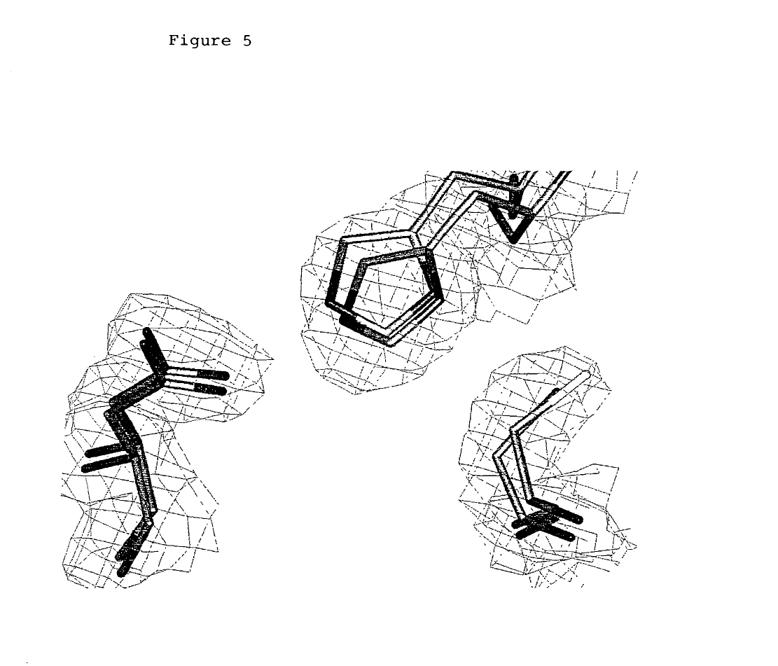

As the two main catalytic residues are part of opposing walls of the catalytic cleft, it is very likely that relative movement of the walls modulates the interaction between the cysteine and histidine moieties in the active enzyme, thus playing a role in the catalytic mechanism. The position of the inactive mutant cathepsin S catalytic triad side chains superimposes with the catalytic triad of the active cathepsin K cysteine residue (compared to the oxygen in the cathepsin S Cys25→Ser serine hydroxyl) that imparts proteolytic activity in the observed structural context (FIG. 5). The pKₐ of the active site cysteine in papain-like proteinases has been measured at 4.5 (Rullmann et al. (1989) J Mol Biol 206(1): 101-18) which is far lower than that expected for serine in the equivalent position. This allows a proton to be pulled from the cysteine sulphidyl by the Asn polarized active site His, whereas a catalytic triad aspartic acid residue is required for the histidine polarization necessary for proton abstraction from a serine residue (Tsukada and Blow (1985) J Mol Biol 184(4): 703-11).

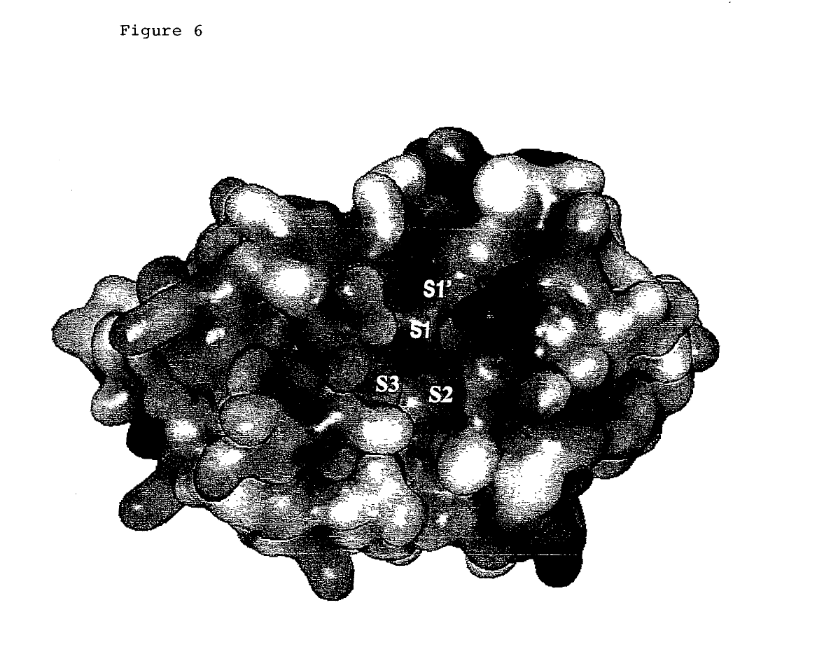

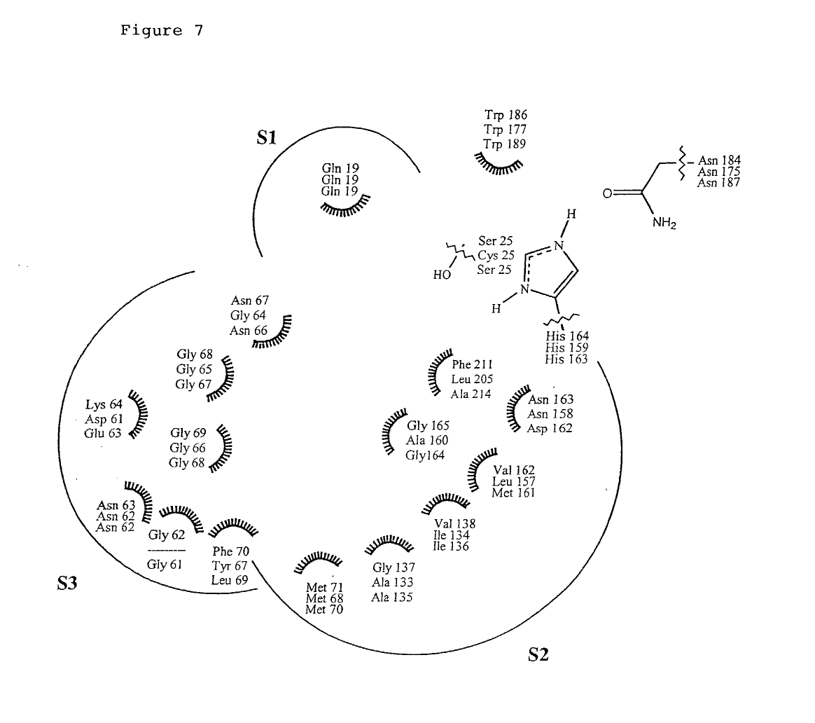

The substrate binding site, formed between the two domains, extends on either side of the catalytic residues, with three binding pockets S3, S2 and S 1 for substrate residues on the amino-terminal side of the scissile bond (Schechter & Berger (1967) Biochem Biophys Res Commun 27(2): 157-62) and a clear binding pocket S₁′ and an extended area S₂′ at the substrate carboxyterminal side (FIG. 6). The previously described cathepsin S structure gave a general description of the peptide-binding cleft, but did not show the cathepsin S-specific loop containing residues 58-61. This region of the enzyme can clearly be seen shaping the back of the S3 pocket and has important implications for the design of selective inhibitors. For example, unlike the open cleft S3 binding region found in cathepsin K (Bossard et al. (1999) Biochemistry 38(48): 15983-902); Marquis et al. (2001) J Med Chem 44(9): 1380-1395); Marquis et al. (2001) J Med Chem 44(5): 725-36), cathepsin S has a small pocket which could be exploited for selectivity in drug design, for instance by the use of a small cyclic capping group at the N-terminus of a peptidomimetic inhibitor, such as those elaborated in WO00/69855. The S3 pocket lined by the residues Gly68, Gly69 at the base with Phe70, Gly62, Asn63 and Lys64 forming the sides and rear of the binding region (FIG. 7).

The relatively large open S2 pocket of cathepsin S, which also contributes to enzyme substrate selectivity, has been extensively studied (Bromme et al. (1994) J Biol Chem 269(48): 30238-42; Bromme et al. (1996) Biochem J 315(Pt 1): 85-9; McGrath et al. (1998) Protein Sci 7(6) 1294-302) and is composed of residues Met71, Gly 137, Val138, Val162, Gly165 and Phe211. This region has been shown to prefer branched hydrophobic side chains with Gly133 appearing to provide more space relative to the alanine residue found in the equivalent position in cathepsin K and L (FIG. 7).

B. Uses of the Structure Coordinates of catS

The present invention permits the use of molecular design techniques to design, select and synthesize chemical entities and compounds, including inhibitory compounds, capable of binding to the active site or accessory binding site of catS, in whole or in part.

One approach enabled by this invention, is to use the structure coordinates of catS to design compounds that bind to the enzyme and alter the physical properties of the compounds in different ways, e.g., solubility. For example, this invention enables the design of compounds that act as competitive inhibitors of the catS enzyme by binding to all or a portion of the active site of catS. This invention also enables the design of compounds that act as uncompetitive inhibitors of the catS enzyme. These inhibitors may bind to all or a portion of the accessory binding site of a catS already bound to its substrate and may be more potent and less non-specific than known competitive inhibitors that compete only for the catS active site. Similarly, non-competitive inhibitors that bind to and inhibit catS whether or not it is bound to another chemical entity may be designed using the structure coordinates of catS of this invention.

A second design approach is to probe a catS crystal with molecules composed of a variety of different chemical entities to determine optimal sites for interaction between candidate catS inhibitors and the enzyme. For example, high resolution X-ray diffraction data collected from crystals saturated with solvent allows the determination of where each type of solvent molecule sticks. Small molecules that bind tightly to those sites can then be designed and synthesized and tested for their catS inhibitor activity (Travis (1993) Science 262: 1374).

This invention also enables the development of compounds that can isomerize to short-lived reaction intermediates in the chemical reaction of a substrate or other compound that binds to catS, with catS. Thus, the time-dependent analysis of structural changes in catS during its interaction with other molecules is enabled. The reaction intermediates of catS can also be deduced from the reaction product in co-complex with catS. Such information is useful to design improved analogues of known catS inhibitors or to design novel classes of inhibitors based on the reaction intermediates of the catS enzyme and catS-inhibitor co-complex. This provides a novel route for designing catS inhibitors with both high specificity and stability.

Another approach made possible and enabled by this invention, is to screen computationally small molecule databases for chemical entities or compounds that can bind in whole, or in part, to the catS enzyme. In this screening, the quality of fit of such entities or compounds to the binding site may be judged either by shape complementarity or by estimated interaction energy. Meng et al. (1992) J. Comp. Chem. 13: 505-524).

Because catS may crystallize in more than one crystal form, the structure coordinates of catS, or portions thereof, as provided by this invention are particularly useful to solve the structure of those other crystal forms of catS. They may also be used to solve the structure of catS mutants, CAYS co-complexes, or of the crystalline form of any other protein with significant amino acid sequence homology to any functional domain of catS.

One method that may be employed for this purpose is molecular replacement. In this method, the unknown crystal structure, whether it is another crystal form of catS, a catS mutant, or a catS co-complex, or the crystal of some other protein with significant amino acid sequence homology to any functional domain of catS, may be determined using the catS structure coordinates of this invention as provided in TABLE 3. This method will provide an accurate structural form for the unknown crystal more quickly and efficiently than attempting to determine such information ab initio.

In addition, in accordance with this invention, catS mutants may be crystallized in co-complex with known catS inhibitors, The crystal structures of a series of such complexes may then be solved by molecular replacement and compared with that of wild-type catS. Potential sites for modification within the various binding sites of the enzyme may thus be identified. This information provides an additional tool for determining the most efficient binding interactions, for example, increased hydrophobic interactions, between catS and a chemical entity or compound.

All of the complexes referred to above may be studied using well-known X-ray diffraction techniques and may be refined versus 2-3 Å resolution X-ray data to an R value of about 0.20 or less using computer software, such as X-PLOR (Yale University, copyright 1992, distributed by Molecular Simulations, Inc.). See, e.g., Blundel & Johnson, supra; Methods in Enzymology, vol. 114 & 115, H. W. Wyckoff et al., eds., Academic Press (1985). This information may thus be used to optimize known classes of catS inhibitors, and more importantly, to design and synthesize novel classes of catS inhibitors.

The structure coordinates of catS mutant provided in this invention also facilitates the identification of related proteins or enzymes analogous to catS in function, structure or both, thereby further leading to novel therapeutic modes for treating or preventing autoimmune diseases.

The design of compounds that bind to or inhibit catS according to this invention generally involves consideration of two factors. First, the compound must be capable of physically and structurally associating with catS. Non-covalent molecular interactions important in the association of catS with its substrate include hydrogen bonding, van der Waals and hydrophobic interactions.

Second, the compound must be able to assume a conformation that allows it to associate with catS. Although certain portions of the compound will not directly participate in this association with catS, those portions may still influence the overall conformation of the molecule. This, in turn, may have a significant impact on potency. Such conformational requirements include the overall three-dimensional structure and orientation of the chemical entity or compound in relation to all or a portion of the binding site, e.g., active site or accessory binding site of catS, or the spacing between functional groups of a compound comprising several chemical entities that directly interact with catS.

The potential inhibitory or binding effect of a chemical compound on catS may be analyzed prior to its actual synthesis and testing by the use of computer modeling techniques. If the theoretical structure of the given compound suggests insufficient interaction and association between it and catS, synthesis and testing of the compound is obviated. However, if computer modeling indicates a strong interaction, the molecule may then be synthesized and tested for its ability to bind to catS and inhibit by using the assay of Bromme et al., supra. In this manner, synthesis of inoperative compounds may be avoided. For example, the amino acids used to define the binding pockets are in effect “critical” to the design of an inhibitor which is selective for cathepsin S and yet substantially unreactive to other enzymes of the papain superfamily. Consequently, computer modeling can predict tight interactions between the putative inhibitor and the pocket residues that do not intrude into their respective volumes.

An inhibitory or other binding compound of catS may be computationally evaluated and designed by means of a series of steps in which chemical entities or fragments are screened and selected for their ability to associate with the individual binding pockets or other areas of catS.

One skilled in the art may use one of several methods to screen chemical entities or fragments for their ability to associate with catS and more particularly with the individual binding pockets of the catS active site or accessory binding site. This process may begin by visual inspection of, for example, the active site on the computer screen based on the catS coordinates in TABLE 3. Selected fragments or chemical entities may then be positioned in a variety of orientations, or docked, within an individual binding pocket of catS as defined supra. Docking may be accomplished using software such as Quanta and Sybyl, followed by energy minimization and molecular dynamics with standard molecular mechanics forcefields, such as CHARMM and AMBER.

For example, the Molecular Similarity application permits comparisons between different structures, different conformations of the same structure, and different parts of the same structure. The procedure used in Molecular Similarity to compare structures is divided into four: 1) load the structures to be compared; 2) define the atom equivalences in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention we will define equivalent atoms as protein backbone atoms (N, Ca, C and O) for all conserved residues between the two structures being compared. We will also consider only rigid fitting operations.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex or binding pocket thereof that has a root mean square deviation of conserved residue backbone atoms (N, Ca, C, O) of less than 1.5 Å, preferably between 1.5 and 0.3 Å, more preferably between 1.0 and 0.3 Å, even more preferably between 0.8, 0.75, 0.6 and 0.5, and 0.3 Å when superimposed on the relevant backbone atoms described by structure coordinates listed in TABLE 3 are considered identical. Most preferably, the root mean square deviation is between 0.45, 0.4 or 0.35 and 0.3 Å.

Therefore, according to one embodiment, the present invention provides a molecule or molecular complex comprising all or any parts of the binding pocket defined by structure coordinates of catS amino acids, i.e., amino acids 19, 23-26, 62-64, 67, 68, 69, 70, 71, 137, 138, 162, 163, 164, 165, 186 and/or 211 according to TABLE 3, or a homologue of said molecule or molecular complex, wherein said homologue comprises a binding pocket that has a root mean square deviation from the backbone atoms of said amino acids of not more than 1.5 Å.

More preferred are molecules or molecular complexes that are defined by the entire set of structure coordinates in TABLE 3 +/− a root mean square deviation from the conserved backbone atoms of those amino acids of not more than 1.5 Å.

In order to use the structure coordinates generated for the catS enzyme, one of its binding pockets or homologues thereof, it is sometimes necessary to convert them into a three-dimensional shape. This may be achieved through the use of commercially available software that is capable of generating three-dimensional graphical representations of molecules or portions thereof from a set of structure coordinates.

Therefore, according to another embodiment, the present invention provides an apparatus and a method for generating and displaying a graphical three-dimensional representation of a molecule or molecular complex comprising all or any parts of a binding pocket defined by structure coordinates of catS amino acids, i.e., amino acids 19, 23-26, 62-64, 67, 68, 69, 70, 71, 137, 138, 162, 163, 164, 165, 186 and/or 211 according to TABLE 3, or a homologue of said molecule or molecular complex, wherein said homologue comprises a binding pocket that has a root mean square deviation from the backbone atoms of said amino acids of not more than 1.5 Å. According to another embodiment, the present invention provides a machine-readable data storage medium, comprising a data storage material encoded with machine readable instructions which are executable by a machine to generate and display a graphical three-dimensional representation of a molecule or molecular complex comprising all or any parts of a binding pocket defined by structure coordinates of catS amino acids, i.e., amino acids 19, 23-26, 62-64, 67, 68, 69, 70, 71, 137, 138, 162, 163, 164, 165, 186 and/or 211 according to TABLE 3, or a homologue of said molecule or molecular complex, wherein said homologue comprises a binding pocket that has a root mean square deviation from the backbone atoms of said amino acids of not more than 1.5 Å.

Even more preferred is a machine-readable data storage medium that stores encoded machine readable instructions that are executable by a machine to generate and display a graphical three-dimensional representation of a molecule or molecular complex that is defined by the structure coordinates of all of the amino acids in TABLE 3 +/− a root mean square deviation from the backbone atoms of those amino acids of not more than 1.5 Å.

According to an alternate embodiment, the machine-readable data storage medium comprises a data storage material encoded with a first set of machine readable data which comprises the Fourier transform of the structure coordinates set forth in TABLE 3, and which, when accessed by a machine programmed with instructions for using said data, can be combined with a second set of machine readable data comprising the X-ray diffraction pattern of a molecule or molecular complex to determine at least a portion of the structure coordinates corresponding to the second set of machine readable data.

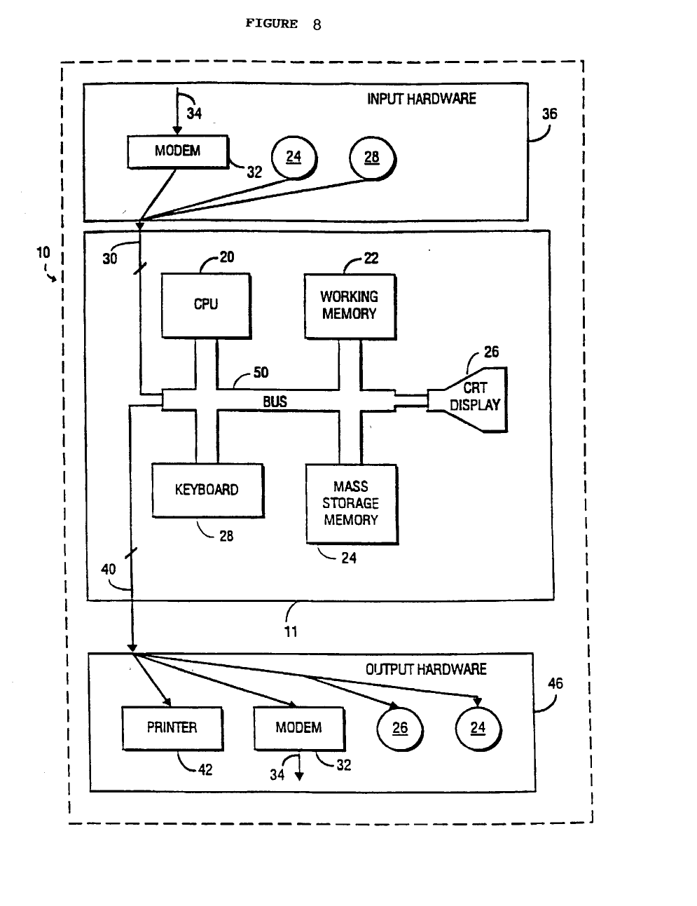

FIG. 8 demonstrates one version of these embodiments. System 10 includes a computer 11 comprising a central processing unit (“CPU”) 20, a working memory 22 which may be, e.g, RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding pocket of this invention using a program such as QUANTA as described herein. Output hardware might also include a printer 42, so that hard copy output may be produced, or a disk drive 24, to store system output for later use.

In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

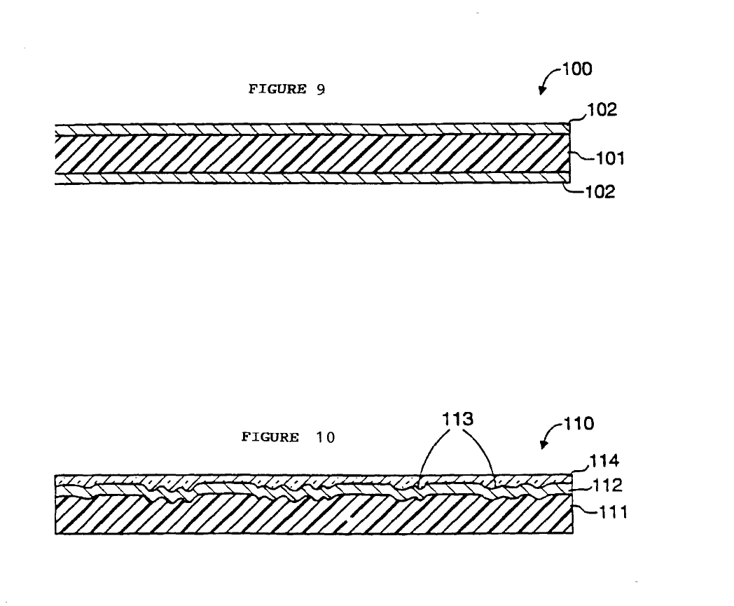

FIG. 9 shows a cross section of a magnetic data storage medium 100 which can be encoded with a machine-readable data that can be carried out by a system such as system 10 of FIG. 8. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24.

The magnetic domains of coating 102 of medium 100 are polarized or oriented so as to encode in manner which may be conventional, machine readable data such as that described herein, for execution by a system such as system 10 of FIG. 8.

FIG. 10 shows a cross section of an optically-readable data storage medium 110 which also can be encoded with such a machine-readable data, or set of instructions, which can be carried out by a system such as system 10 of FIG. 9. Medium 110 can be a conventional compact disk read only memory (CD-ROM) or a rewritable medium such as a magneto-optical disk which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

Thus, in accordance an embodiment of the present invention, data capable of displaying the three dimensional structure of catS and portions thereof and their structurally similar homologues is stored in a machine-readable storage medium, which also stores instructions for using such data that are executable by a machine to display a graphical three-dimensional representation of the structure. Such data may be used for a variety of purposes, such as drug discovery.

For example, the structure encoded by the data may be computationally evaluated for its ability to associate with chemical entities. Chemical entities that associate with catS may inhibit catS, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential of a chemical entity to associate with any of the molecules or molecular complexes set forth above. This method comprises the steps of: a) employing computational means to perform a fitting operation between the chemical entity and a binding pocket of the molecule or molecular complex; and b) analyzing the results of said fitting operation to quantify the association between the chemical entity and the binding pocket. The term “chemical entity”, as used herein, refers to chemical compounds, complexes of at least two chemical compounds, and fragments of such compounds or complexes.

For the first time, the present invention permits the use of molecular design techniques to identify, select and design chemical entities, including inhibitory compounds, capable of binding to catS-like binding pockets.

Applicants' elucidation of the binding sites on catS provides the necessary information for designing new chemical entities and compounds that may interact with at least one catS-like binding pockets, in whole or in part. This elucidation also enables the evaluation of structure-activity data for analogs of MPA or other compounds which bind to catS-like binding pockets.

Throughout this section, discussions about the ability of an entity to bind to, associate with or inhibit a catS-like binding pocket refers to features of the entity alone. Assays to determine if a compound binds to catS are disclosed in EurJ 1997250:745-750, WO00/69855, WO01/9808, WO0119796, and WO0109160 and other patent specifications relating to cathepsin S inhibitors The design of compounds that bind to or inhibit catS-like binding pockets according to this invention generally involves consideration of two factors. First, the entity must be capable of physically and structurally associating with parts or all of the catS-like binding pockets. Non-covalent molecular interactions important in this association include hydrogen bonding, van der Waals interactions, hydrophobic interactions and electrostatic interactions.

Second, the entity must be able to assume a conformation that allows it to associate with the catS-like binding pocket directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the catS-like binding pocket or homologues thereof.

The potential inhibitory or binding effect of a chemical entity on a catS-like binding pocket may be analyzed prior to its actual synthesis and testing by the use of computer modeling techniques. If the theoretical structure of the given entity suggests insufficient interaction and association between it and the catS-like binding pocket, testing of the entity is obviated. However, if computer modeling indicates a strong interaction, the molecule may then be synthesized and tested for its ability to bind to a catS-like binding pocket. This may be achieved by testing the ability of the molecule to inhibit catS using the assays described in the Examples. In this manner, synthesis of inoperative compounds may be avoided.

A potential inhibitor of a catS-like binding pocket may be computationally evaluated by means of a series of steps in which chemical entities or fragments are screened and selected for their ability to associate with the catS binding pockets.

One skilled in the art may use one of several methods to screen chemical entities or fragments for their ability to associate with a catS-like binding pocket. This process may begin by visual inspection of, for example, a catS-like binding pocket on the computer screen based on the catS structure coordinates in TABLE 3 or other coordinates which define a similar shape generated from the machine-readable storage medium. Selected fragments or chemical entities may then be positioned in a variety of orientations, or docked, within that binding pocket as defined supra. Docking may be accomplished using software such as Quanta and Sybyl, followed by energy minimization and molecular dynamics with standard molecular mechanics force fields, such as CHARMM and AMBER.

Specialized computer programs may also assist in the process of selecting fragments or chemical entities. These include:

1. GRID (Goodford (1985)“A Computational Procedure for Determining Energetically Favorable Binding Sites on Biologically Important Macromolecules”, J. Med. Chem 28: 849-857). GRID is available from Oxford University, Oxford, UK.

2. MCSS (Miranker and Karplus (1991) “Functionality Maps of Binding Sites: A Multiple Copy Simultaneous Search Method.” Proteins: Structure. Function and Genetics, 11, pp. 29-34). MCSS is available from Molecular Simulations, Burlington, Mass.

3. AUTODOCK (Goodsell and Olsen (1990) “Automated Docking of Substrates to Proteins by Simulated Annealing”, Proteins: Structure. Function, and Genetics, 8, pp. 195-202). AUTODOCK is available from Scripps Research Institute, La Jolla, Calif.

4. DOCK (Kuntz et al. (1982) “A Geometric Approach to Macromolecule-Ligand Interactions”, J. Mol. Biol. 161: 269-288). DOCK is available from University of California, San Francisco, Calif.

Once suitable chemical entities or fragments have been selected, they can be assembled into a single compound or inhibitor. Assembly may be proceed by visual inspection of the relationship of the fragments to each other on the three-dimensional image displayed on a computer screen in relation to the structure coordinates of catS. This would be followed by manual model building using software such as Quanta or Sybyl.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of catS. This would be followed by manual model building using software such as Quanta or Sybyl [Tripos Associates, St. Louis, Mo.].

Useful programs to aid one of skill in the art in connecting the individual chemical entities or fragments include:

1. CAVEAT (Bartlett et al. (1989) “CAVEAT: A Program to Facilitate the Structure-Dervived Design of Biologically Active Molecules”. In “Molecular Recognition in Chemical and Biological Problems”, Special Pub., Royal Chem. Soc. 78: 182-196). CAVEAT is available from the University of California, Berkeley, Calif.

2. 3D Database systems such as MACCS-3D and ISIS (MDL Information Systems, San Leandro, Calif.). This area is reviewed in Martin (1992) “3D Database Searching in Drug Design”, J. Med. Chem. 35: 2145-2154).

3. HOOK (available from Molecular Simulations, Burlington, Mass.).

4. SPROUT (V. Gillet et al. (1993) “SPROUT: A Program for Structure Generation,” J. Comput. Aided Mol. Design, 7: 127-153.). SPROUT is available from the University of Leeds, UK.

Instead of proceeding to build a catS inhibitor in a step-wise fashion one fragment or chemical entity at a time as described above, inhibitory or other catS binding compounds may be designed as a whole or “de novo” using either an empty active site or optionally including some portion(s) of a known inhibitor(s). These methods include:

1. LUDI (Bohm (1992) “The Computer Program LUDI: A New Method for the De Novo Design of Enzyme Inhibitors”, J. Comp. Aid. Molec. Design 6: 61-78). LUDI is available from Biosym Technologies, San Diego, Calif.

2. LEGEND (Nishibata and Itai (1991) Tetrahedron 47: 8985). LEGEND is available from Molecular Simulations, Burlington, Mass.

3. LeapFrog (available from Tripos Associates, St. Louis, Mo.).

Other molecular modeling techniques may also be employed in accordance with this invention. See, e.g., Cohen, N. C. et al., “Molecular Modeling Software and Methods for Medicinal Chemistry”, J. Med. Chem., 33, pp. 883-894 (1990). See also, Navia and Murcko, (1992)“The Use of Structural Information in Drug Design”, Current Opinions in Structural Biology 2: 202-210.

Once a compound has been designed or selected by the above methods, the efficiency with which that compound may bind to catS may be tested and optimized by computational evaluation. For example, a compound that has been designed or selected to function as an catS-inhibitor must also preferably traverse a volume not overlapping that occupied by the active site when it is bound to the native substrate. An effective catS inhibitor must preferably demonstrate a relatively small difference in energy between its bound and free states (i.e., a small deformation energy of binding). Thus, the most efficient catS inhibitors should preferably be designed with a deformation energy of binding of not greater than about 10 kcal/mole, preferably, not greater than 7 kcal/mole. catS inhibitors may interact with the enzyme in more than one conformation that is similar in overall binding energy. In those cases, the deformation energy of binding is taken to be the difference between the energy of the free compound and the average energy of the conformations observed when the inhibitor binds to the enzyme.

A compound designed or selected as binding to catS may be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the inhibitor and the enzyme when the inhibitor is bound to catS, preferably make a neutral or favorable contribution to the enthalpy of binding.

Specific computer software is available in the art to evaluate compound deformation energy and electrostatic interaction. Examples of programs designed for such uses include: Gaussian 92, revision C—M. J. Frisch, Gaussian, Inc., Pittsburgh, Pa. ©1992; AMBER, version 4.0—P. A. Kollman, University of California at San Francisco, ©1994!; QUANTA/CHARMM—Molecular Simulations, Inc., Burlington, Mass. ©1994; and Insight II/Discover (Biosysm Technologies Inc., San Diego, Calif. ©1994). These programs may be implemented, for instance, using a Silicon Graphics workstation, IRIS 4D/35 or IBM RISC/6000 workstation model 550. Other hardware systems and software packages will be known to those skilled in the art.

Once a catS-binding compound has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to catS by the same computer methods described in detail, above.

Another approach enabled by this invention, is the computational screening of small molecule databases for chemical entities or compounds that can bind in whole, or in part, to a catS binding pocket. In this screening, the quality of fit of such entities to the binding site may be judged either by shape complementarity or by estimated interaction energy (E. C. Meng et al. (1992) J. Comp. Chem. 13: 505-524). Thus, enabled by this invention are compounds that inhibit catS by associating directly with the CAT binding site.

The term “immunosuppressant” refers to a compound or drug that possesses immune response inhibitory activity. Examples of such agents include cyclosporin A, FK506, rapamycin, leflunomide, deoxyspergualin, prednisone, azathioprine, mycophenolate mofetil, OKT3, ATAG and mizoribine.

catS-mediated disease refers to any disease state in which the catS enzyme plays a regulatory role in the metabolic pathway of that disease. Examples of catS-mediated disease include rheumatoid arthritis, asthma, atherosclerosis, COPD and multiple sclerosis. See also WO97/40066 relating to the identification of the role of cathepsin S in MHC processing.

The invention allows the identification and characterization of catS inhibitors, which will typically comprise a catS inhibitor as identified and characterized herein or a pharmaceutically acceptable salt thereof; and a pharmaceutically acceptable carrier, adjuvant or vehicle. Such composition may optionally comprise an additional agent selected from an immunosuppressant, an anti-cancer agent, an anti-viral agent, or an anti-vascular hyperproliferation compound.

C. Mutants of catS

The present invention also enables mutants of catS and the solving of their crystal structure. More particularly, by virtue of the present invention, the location of the active site, accessory binding site and interface of catS based on its crystal structure permits the identification of desirable sites for mutation.

For example, mutation may be directed to a particular site or combination of sites of wild-type catS, i.e., the accessory binding site or only the active site, or a location on the interface site may be chosen for mutagenesis. Similarly, only a location on, at or near the enzyme surface may be replaced, resulting in an altered surface charge of one or more charge units, as compared to the wild-type enzyme. Alternatively, an amino acid residue in catS may be chosen for replacement based on its hydrophilic or hydrophobic characteristics.

Such mutants may be characterized by any one of several different properties as compared with wild-type catS. For example, such mutants may have altered surface charge of one or more charge units, or have an increased stability to subunit dissociation. Or such mutants may have an altered substrate specificity in comparison with, or a higher specific activity than, wild-type ICE.

The mutants of catS prepared by this invention may be prepared in a number of ways. For example, the wild-type sequence of catS may be mutated in those sites identified using this invention as desirable for mutation, by means of oligonucleotide-directed mutagenesis or other conventional methods, e.g. deletion. Alternatively, mutants of catS may be generated by the site specific replacement of a particular amino acid with an unnaturally occurring amino acid. In addition, catS mutants may be generated through replacement of an amino acid residue, or a particular cysteine or methionine residue, with selenocysteine or selenomethionine. This may be achieved by growing a host organism capable of expressing either the wild-type or mutant polypeptide on a growth medium depleted of either natural cysteine or methionine (or both) but enriched in selenocysteine or selenomethionine (or both).

Mutations may be introduced into a DNA sequence coding for catS using synthetic oligonucleotides. These oligonucleotides contain nucleotide sequences flanking the desired mutation sites. Mutations may be generated in the full-length DNA sequence of catS (SEQ ID: 1).

According to this invention, a mutated catS DNA sequence produced by the methods described above, or any alternative methods known in the art, can be expressed using an expression vector. An expression vector, as is well known in the art, typically includes elements that permit autonomous replication in a host cell independent of the host genome, and one or more phenotypic markers for selection purposes. Either prior to or after insertion of the DNA sequences surrounding the desired catS mutant coding sequence, an expression vector also will include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” may be inserted prior to the catS mutant coding sequence. For expression under the direction of the control sequences, a desired DNA sequence must be operatively linked to the control sequences. That is, they must have an appropriate start signal in front of the DNA sequence encoding the catS mutant and must maintain the correct reading frame to permit expression of that sequence under the control of the control sequences and production of the desired product encoded by that catS sequence.

Any of a wide variety of well known available expression vectors are useful to express the mutated catS coding sequences of this invention.

These include, for example, vectors consisting of segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage lambda, e.g., NM 989, and other DNA phages, e.g., M13 and filamentous single stranded DNA phages, yeast plasmids such as the 282 plasmid or derivatives thereof, and vectors derived from combinations of plasmids and phage DNAs, such as plasmids which have been modified to employ phage DNA or other expression control sequences. In the preferred embodiments of this invention, we employ baculovirus vectors in Sf9 insect cells.

In addition, any of a wide variety of expression control sequences (I.e. sequences that control the expression of a DNA sequence when operatively linked to it) may be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage lambda the control regions of fd coat protein, all for E. coli, the promoter for 3-phosphoglycerate kinase or other glycolytic enzymes, the promoters of acid phosphatase, e.g., Pho5, the promoters of the yeast α-mating factors for yeast, and, other sequences known to control the expression of genes of prokaryotic or eukaryotic cells or their viruses, and vanous combinations thereof. In the preferred embodiments of this invention, we used baculovirus vectors in Sf9 insect cells.

A wide variety of hosts are also useful for producing mutated catS for this invention. These hosts include, for example, bacteria, such as E. coli, Bacillus and Streptomyces, fungi, such as yeasts, and animal cells, such as CHO and COS-1 cells, plant cells, insect cells and transgenic host cells. In preferred embodiments of this invention, the host cells are Sf9 insect cells.

It should be understood that not all expression vectors and expression systems function in the same way to express mutated DNA sequences of this invention and to produce modified catS or catS mutants. Neither do all hosts function equally well with the same expression system. However, one of skill in the art may make a selection among these vectors, expression control sequences and hosts without undue experimentation and without departing from the scope of this invention. For example, an important consideration in selecting a vector, will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability, its compatibility with the DNA sequence encoding the modified catS of this invention, particularly with regard to potential secondary structures.

Hosts should be selected by consideration of their compatibility with the chosen vector, the toxicity of the modified catS to them, their ability to secrete mature products, their ability to fold proteins correctly, their fermentation requirements, the ease of the purification of the modified catS from them and safety. Within these parameters, one of skill in the art may select various vector/expression control system/host combinations that will produce useful amounts of the mutant catS.

The mutant catS produced in these systems may be purified by a variety of conventional steps and strategies, including those used to purify wild-type catS.

Once the catS mutants have been generated in the desired location, i.e., active site or accessory binding site, the mutants may be tested for any one of several properties of interest.

For example, mutants may be screened for an altered charge at physiological pH. This is determined by measuring the mutant catS isoelectric point (pI) in comparison with that of the wild-type parent. Isoelectric point may be measured by gel-electrophoresis according to the method of Wellner (1971) Analyt. Chem. 43: 597. A mutant with an altered surface charge is an catS polypeptide containing a replacement amino acid located at the surface of the enzyme, as provided by the structural information of this invention, and an altered pI.

Furthermore, mutants may be screened for altered specific activity in relation to the wild-type catS.

A mutant would be tested for altered catS substrate specificity by measuring the hydrolysis of fluorgenic peptide substrates or unmodified catS peptide substrates such as Abz-Leu-Thr-Bal-Hyp-Tyr(NO₂)-Asp-NH₂.

Further properties of interest also include mutants with a broader range of pH stability. A catS mutant with a broader range of pH stability would demonstrate no loss of enzymatic activity at pH in the range of 5-7.

In order that the invention described herein may be more fully understood, the following examples are set forth. It should be understood that these examples are for illustrative purposes only and are not to be construed as limiting this invention in any manner.

### EXAMPLE 1

Crystal Structure of catS

The cDNA encoding the precursor of active human catS (Shi et al. (1994) J Biol Chem 269(15): 11530-36) was cloned into a baculovirus expression vector and expressed in Sf9 insect cells. The mutant Cys25→Ser was expressed at levels approaching 35 mg/l as pro-enzyme with approximately half secreted and half kept inside the cells (Vernet et al. (1990) J Biol Chem 265(27): 16661-6).

Crystallization was performed by the hanging-drop vapor diffusion method. Equal volumes cathepsin S (Cys25→Ser) at 7 mg/ml, including the peptide as Abz-Leu-Thr-Bal-Hyp-Tyr(NO₂)-Asp-NH₂ at 1 mM and well solution were combined and placed over a well containing 20% isopropanol, 20% PEG 2000 and 0.1 M sodium citrate, pH 4.13. Rod-shaped crystals appeared after 10 days.

Those of skill in the art will appreciate that the aforesaid crystallization conditions can be varied. Such variations may be used alone or in combination, and include final protein/inhibitor complex; all combinations of catS/inhibitor to precipitant ratios; citrate concentrations between 0.01 mM and 200 mM; any concentration of β-mercaptoethanol; pH ranges between 4.0 and 9.5; PEG concentrations between 10% and 25% (g/100 ml); PEG weights between 2000 and 8000; any concentration or type of detergent; any temperature between −5° C. and 300° C. and crystallization of catS/inhibitor complexes by batch, liquid bridge, or dialysis method using these conditions or variations thereof.

The cathepsin S (Cys25→Ser) data were collected from a crystal with dimensions of 0.2×0.1×0.1 mm³ using a MAR345 imaging plate detector, mounted on a RU-H3R Rigaku rotating anode X-ray generator operating at 50 kV and 100 mA, equipped with Osmic multilayers. The data were processed using the programs DENZO and SCALEPACK (Otwinowsi & Minor, 1997). The crystals belong to the trigonal space group P3121 with a=b80.0 Å, c=61.5 Å. Assuming one protein molecule in the asymmetric unit, the Matthews coefficient is 2.3 Å³/Da, corresponding to a solvent content of 46%. Data statistics are given in TABLE 1.

TABLE 1
Data collection and refinement statistics

| | |
|---|---|
| Space group | P3₁2I |
| Resolution range (Å) | 20-2.2 |
| Outer resolution shell (Å) | 2.24-2.20 |
| Rsym (%): overall (outer shell) | 7.5 (27.0) |
| Completeness (%): overall | 99.4 (98.8) |
| (outer shell) | |
| Number of observations | 39038 |
| Number of unique reflections | 11969 |
| Cross-validation method | Throughout |
| Free R value test set selection | Random |
| R value (working + test set) | 0.197 |
| R value (working) | 0.194 |
| Free R value | 0.251 |
| Free R value test set size (%) | 5.0 |
| Number of reflections in Free | 597 |
| R value test set | |
| Number of non-hydrogen atoms | 1841 |
| used in refinement | |

Model Statistics

| | RMS | SIGMA |
|---|---|---|
| Bond length (Å) | 0.013 | 0.021 |
| Bond angle (°) | 1.754 | 1.942 |
| Torsion angles, period 1(°) | 4.176 | 3.0 |
| Torsion angles, period 3(°) | 17.488 | 15.0 |
| Chiral-center restraints (Å) | 0.121 | 0.20 |
| Plane restrain (Å) | 0.006 | 0.020 |
| VDW repulsions (Å) | 0.234 | 0.30 |
| Hbonds (Å) | 0.141 | 0.50 |

The following Tables (TABLE 2 and 3) summarize the crystallographic coordinate transformation data and the X-ray crystallography data sets of catS derivatives that were used to determine the structure of catS according to this invention.

TABLE 2
Crystallographic Coordinate Transformation Data

| Record | | | | alpha | beta | gamma | Space |
|---|---|---|---|---|---|---|---|
| name | a (Angstroms) | b (Angstroms) | c (Angstroms) | (degrees) | (degrees) | (degrees) | group |
| Cryst 1 | 79.989 | 79.989 | 61.517 | 90.00 | 90.00 | 120.00 | P3121 |

| Record | | | | |
|---|---|---|---|---|
| name | Sn1 | Sn2 | Sn3 | Un |
| SCALE1 | 0.012502 | 0.007218 | 0.000000 | 0.00000 |
| SCALE2 | 0.000000 | 0.014436 | 0.000000 | 0.00000 |
| SCALE3 | 0.000000 | 0.000000 | 0.016256 | 0.00000 |

TABLE 3

| | | Atom | | Chn | Res. | | | | | Temp | Atomic | Element |
|---|---|---|---|---|---|---|---|---|---|---|---|---|
| Description | # | Type | Res. | I.D. | # | X coord. | Y coord. | Z coord. | Occ. | fact | # | symbol |
| ATOM | 1 | N | LEU | A | 1 | 10.950 | 18.311 | 9.373 | 1.00 | 37.95 | 7 | N |
| ATOM | 2 | CA | LEU | A | 1 | 11.173 | 17.084 | 8.521 | 1.00 | 37.35 | 6 | C |
| ATOM | 3 | C | LEU | A | 1 | 10.316 | 17.197 | 7.273 | 1.00 | 35.18 | 6 | C |
| ATOM | 4 | O | LEU | A | 1 | 10.782 | 17.443 | 6.142 | 1.00 | 34.02 | 8 | O |
| ATOM | 5 | CB | LEU | A | 1 | 10.949 | 15.891 | 9.439 | 1.00 | 40.82 | 6 | C |
| ATOM | 6 | CG | LEU | A | 1 | 11.373 | 14.476 | 9.009 | 1.00 | 42.31 | 6 | C |
| ATOM | 7 | CD1 | LEU | A | 1 | 11.105 | 13.512 | 10.187 | 1.00 | 43.06 | 6 | C |
| ATOM | 8 | CD2 | LEU | A | 1 | 10.488 | 14.042 | 7.823 | 1.00 | 43.00 | 6 | C |
| ATOM | 9 | N | PRO | A | 2 | 9.003 | 17.036 | 7.402 | 1.00 | 32.83 | 7 | N |
| ATOM | 10 | CA | PRO | A | 2 | 8.118 | 17.157 | 6.249 | 1.00 | 30.90 | 6 | C |
| ATOM | 11 | C | PRO | A | 2 | 8.209 | 18.546 | 5.633 | 1.00 | 30.00 | 6 | C |
| ATOM | 12 | O | PRO | A | 2 | 8.489 | 19.556 | 6.291 | 1.00 | 28.06 | 8 | O |
| ATOM | 13 | CB | PRO | A | 2 | 6.751 | 16.801 | 6.783 | 1.00 | 31.24 | 6 | C |
| ATOM | 14 | CG | PRO | A | 2 | 6.860 | 16.525 | 8.231 | 1.00 | 30.69 | 6 | C |
| ATOM | 15 | CD | PRO | A | 2 | 8.285 | 16.712 | 8.650 | 1.00 | 32.33 | 6 | C |
| ATOM | 16 | N | ASP | A | 3 | 7.968 | 18.698 | 4.330 | 1.00 | 28.88 | 7 | N |
| ATOM | 17 | CA | ASP | A | 3 | 8.043 | 19.999 | 3.683 | 1.00 | 28.98 | 6 | C |
| ATOM | 18 | C | ASP | A | 3 | 6.816 | 20.902 | 3.874 | 1.00 | 27.92 | 6 | C |
| ATOM | 19 | O | ASP | A | 3 | 6.918 | 22.121 | 3.725 | 1.00 | 26.64 | 8 | O |
| ATOM | 20 | CB | ASP | A | 3 | 8.273 | 19.889 | 2.184 | 1.00 | 29.50 | 6 | C |
| ATOM | 21 | CG | ASP | A | 3 | 9.737 | 19.805 | 1.814 | 1.00 | 31.71 | 6 | C |
| ATOM | 22 | OD1 | ASP | A | 3 | 10.629 | 19.915 | 2.671 | 1.00 | 33.75 | 8 | O |
| ATOM | 23 | OD2 | ASP | A | 3 | 9.927 | 19.611 | 0.597 | 1.00 | 30.01 | 8 | O |
| ATOM | 24 | N | SER | A | 4 | 5.677 | 20.319 | 4.140 | 1.00 | 27.22 | 7 | N |
| ATOM | 25 | CA | SER | A | 4 | 4.417 | 21.007 | 4.366 | 1.00 | 27.40 | 6 | C |
| ATOM | 26 | C | SER | A | 4 | 3.645 | 20.233 | 5.431 | 1.00 | 25.48 | 6 | C |
| ATOM | 27 | O | SER | A | 4 | 3.810 | 19.015 | 5.387 | 1.00 | 23.18 | 8 | O |
| ATOM | 28 | CB | SER | A | 4 | 3.546 | 20.951 | 3.102 | 1.00 | 27.47 | 6 | C |
| ATOM | 29 | OG | SER | A | 4 | 4.117 | 21.709 | 2.054 | 1.00 | 31.75 | 8 | O |
| ATOM | 30 | N | VAL | A | 5 | 2.972 | 20.875 | 6.374 | 1.00 | 25.14 | 7 | N |
| ATOM | 31 | CA | VAL | A | 5 | 2.104 | 20.188 | 7.292 | 1.00 | 24.37 | 6 | C |
| ATOM | 32 | C | VAL | A | 5 | 0.837 | 21.052 | 7.446 | 1.00 | 23.65 | 6 | C |
| ATOM | 33 | O | VAL | A | 5 | 0.893 | 22.267 | 7.551 | 1.00 | 22.57 | 8 | O |
| ATOM | 34 | CB | VAL | A | 5 | 2.546 | 19.847 | 8.713 | 1.00 | 26.17 | 6 | C |
| ATOM | 35 | CG1 | VAL | A | 5 | 3.532 | 18.649 | 8.728 | 1.00 | 27.21 | 6 | C |
| ATOM | 36 | CG2 | VAL | A | 5 | 3.140 | 21.024 | 9.444 | 1.00 | 26.93 | 6 | C |
| ATOM | 37 | N | ASP | A | 6 | −0.262 | 20.319 | 7.419 | 1.00 | 22.38 | 7 | N |
| ATOM | 38 | CA | ASP | A | 6 | −1.544 | 21.000 | 7.641 | 1.00 | 22.53 | 6 | C |
| ATOM | 39 | C | ASP | A | 6 | −2.250 | 20.091 | 8.639 | 1.00 | 22.05 | 6 | C |
| ATOM | 40 | O | ASP | A | 6 | −2.660 | 18.980 | 8.321 | 1.00 | 19.08 | 8 | O |
| ATOM | 41 | CB | ASP | A | 6 | −2.260 | 21.289 | 6.338 | 1.00 | 22.86 | 6 | C |
| ATOM | 42 | CG | ASP | A | 6 | −3.545 | 22.053 | 6.618 | 1.00 | 24.23 | 6 | C |
| ATOM | 43 | OD1 | ASP | A | 6 | −3.824 | 22.335 | 7.803 | 1.00 | 25.03 | 8 | O |
| ATOM | 44 | OD2 | ASP | A | 6 | −4.270 | 22.370 | 5.668 | 1.00 | 25.31 | 8 | O |
| ATOM | 45 | N | TRP | A | 7 | −2.383 | 20.593 | 9.874 | 1.00 | 21.56 | 7 | N |
| ATOM | 46 | CA | TRP | A | 7 | −2.985 | 19.811 | 10.953 | 1.00 | 21.32 | 6 | C |
| ATOM | 47 | C | TRP | A | 7 | −4.496 | 19.684 | 10.796 | 1.00 | 21.32 | 6 | C |
| ATOM | 48 | O | TRP | A | 7 | −5.159 | 18.823 | 11.383 | 1.00 | 20.11 | 8 | O |
| ATOM | 49 | CB | TRP | A | 7 | −2.609 | 20.350 | 12.338 | 1.00 | 20.99 | 6 | C |
| ATOM | 50 | CG | TRP | A | 7 | −1.244 | 19.901 | 12.774 | 1.00 | 22.17 | 6 | C |
| ATOM | 51 | CD1 | TRP | A | 7 | −0.078 | 20.606 | 12.748 | 1.00 | 22.04 | 6 | C |
| ATOM | 52 | CD2 | TRP | A | 7 | −0.914 | 18.612 | 13.309 | 1.00 | 23.17 | 6 | C |
| ATOM | 53 | NE1 | TRP | A | 7 | 0.955 | 19.850 | 13.229 | 1.00 | 23.04 | 7 | N |
| ATOM | 54 | CE2 | TRP | A | 7 | 0.474 | 18.614 | 13.578 | 1.00 | 23.29 | 6 | C |
| ATOM | 55 | CE3 | TRP | A | 7 | −1.651 | 17.456 | 13.582 | 1.00 | 23.22 | 6 | C |
| ATOM | 56 | CZ2 | TRP | A | 7 | 1.128 | 17.509 | 14.108 | 1.00 | 22.89 | 6 | C |
| ATOM | 57 | CZ3 | TRP | A | 7 | −0.997 | 16.359 | 14.117 | 1.00 | 24.30 | 6 | C |
| ATOM | 58 | CH2 | TRP | A | 7 | 0.390 | 16.388 | 14.372 | 1.00 | 23.98 | 6 | C |
| ATOM | 59 | N | ARG | A | 8 | −5.068 | 20.457 | 9.891 | 1.00 | 21.33 | 7 | N |
| ATOM | 60 | CA | ARG | A | 8 | −6.469 | 20.333 | 9.512 | 1.00 | 22.68 | 6 | C |
| ATOM | 61 | C | ARG | A | 8 | −6.623 | 18.974 | 8.822 | 1.00 | 22.92 | 6 | C |
| ATOM | 62 | O | ARG | A | 8 | −7.619 | 18.297 | 9.110 | 1.00 | 20.36 | 8 | O |
| ATOM | 63 | CB | ARG | A | 8 | −6.916 | 21.554 | 8.709 | 1.00 | 23.11 | 6 | C |
| ATOM | 64 | CG | ARG | A | 8 | −6.693 | 22.889 | 9.419 | 1.00 | 25.30 | 6 | C |
| ATOM | 65 | CD | ARG | A | 8 | −6.997 | 24.066 | 8.512 | 1.00 | 26.01 | 6 | C |
| ATOM | 66 | NE | ARG | A | 8 | −6.153 | 24.141 | 7.326 | 1.00 | 25.53 | 7 | N |
| ATOM | 67 | CZ | ARG | A | 8 | −6.430 | 24.949 | 6.296 | 1.00 | 24.61 | 6 | C |
| ATOM | 68 | NH1 | ARG | A | 8 | −7.506 | 25.723 | 6.302 | 1.00 | 22.60 | 7 | N |
| ATOM | 69 | NH2 | ARG | A | 8 | −5.581 | 24.922 | 5.275 | 1.00 | 24.29 | 7 | N |
| ATOM | 70 | N | GLU | A | 9 | −5.632 | 18.455 | 8.098 | 1.00 | 22.56 | 7 | N |
| ATOM | 71 | CA | GLU | A | 9 | −5.717 | 17.154 | 7.462 | 1.00 | 23.87 | 6 | C |
| ATOM | 72 | C | GLU | A | 9 | −5.719 | 15.981 | 8.429 | 1.00 | 23.76 | 6 | C |
| ATOM | 73 | O | GLU | A | 9 | −6.292 | 14.947 | 8.065 | 1.00 | 22.03 | 8 | O |
| ATOM | 74 | CB | GLU | A | 9 | −4.619 | 16.995 | 6.375 | 1.00 | 24.23 | 6 | C |
| ATOM | 75 | CG | GLU | A | 9 | −4.869 | 18.024 | 5.288 | 1.00 | 24.59 | 6 | C |
| ATOM | 76 | CD | GLU | A | 9 | −3.798 | 18.357 | 4.279 | 1.00 | 26.81 | 6 | C |
| ATOM | 77 | OE1 | GLU | A | 9 | −2.656 | 17.883 | 4.330 | 1.00 | 25.19 | 8 | O |
| ATOM | 78 | OE2 | GLU | A | 9 | −4.135 | 19.162 | 3.356 | 1.00 | 27.08 | 8 | O |
| ATOM | 79 | N | LYS | A | 10 | −5.363 | 16.163 | 9.697 | 1.00 | 23.11 | 7 | N |
| ATOM | 80 | CA | LYS | A | 10 | −5.396 | 15.080 | 10.670 | 1.00 | 25.39 | 6 | C |
| ATOM | 81 | C | LYS | A | 10 | −6.602 | 15.171 | 11.585 | 1.00 | 24.35 | 6 | C |
| ATOM | 82 | O | LYS | A | 10 | −6.658 | 14.490 | 12.608 | 1.00 | 24.83 | 8 | O |
| ATOM | 83 | CB | LYS | A | 10 | −4.086 | 15.098 | 11.504 | 1.00 | 26.85 | 6 | C |
| ATOM | 84 | CG | LYS | A | 10 | −2.957 | 14.583 | 10.612 | 1.00 | 30.32 | 6 | C |
| ATOM | 85 | CD | LYS | A | 10 | −1.565 | 14.890 | 11.082 | 1.00 | 33.59 | 6 | C |
| ATOM | 86 | CE | LYS | A | 10 | −0.899 | 13.748 | 11.846 | 1.00 | 36.10 | 6 | C |
| ATOM | 87 | NZ | LYS | A | 10 | 0.582 | 13.993 | 11.925 | 1.00 | 36.86 | 7 | N |
| ATOM | 88 | N | GLY | A | 11 | −7.533 | 16.056 | 11.251 | 1.00 | 23.07 | 7 | N |
| ATOM | 89 | CA | GLY | A | 11 | −8.729 | 16.279 | 12.072 | 1.00 | 21.28 | 6 | C |
| ATOM | 90 | C | GLY | A | 11 | −8.380 | 16.850 | 13.440 | 1.00 | 19.50 | 6 | C |
| ATOM | 91 | O | GLY | A | 11 | −9.092 | 16.561 | 14.405 | 1.00 | 19.20 | 8 | O |
| ATOM | 92 | N | CYS | A | 12 | −7.376 | 17.719 | 13.580 | 1.00 | 18.82 | 7 | N |
| ATOM | 93 | CA | CYS | A | 12 | −6.932 | 18.176 | 14.903 | 1.00 | 18.52 | 6 | C |
| ATOM | 94 | C | CYS | A | 12 | −7.216 | 19.640 | 15.189 | 1.00 | 19.49 | 6 | C |
| ATOM | 95 | O | CYS | A | 12 | −6.768 | 20.233 | 16.194 | 1.00 | 18.88 | 8 | O |
| ATOM | 96 | CB | CYS | A | 12 | −5.413 | 17.988 | 15.045 | 1.00 | 19.68 | 6 | C |
| ATOM | 97 | SG | CYS | A | 12 | −4.842 | 16.291 | 15.275 | 1.00 | 18.40 | 16 | S |
| ATOM | 98 | N | VAL | A | 13 | −7.941 | 20.266 | 14.262 | 1.00 | 18.11 | 7 | N |
| ATOM | 99 | CA | VAL | A | 13 | −8.236 | 21.686 | 14.335 | 1.00 | 17.21 | 6 | C |
| ATOM | 100 | C | VAL | A | 13 | −9.731 | 21.962 | 14.253 | 1.00 | 17.55 | 6 | C |
| ATOM | 101 | O | VAL | A | 13 | −10.368 | 21.639 | 13.235 | 1.00 | 16.08 | 8 | O |
| ATOM | 102 | CB | VAL | A | 13 | −7.498 | 22.432 | 13.214 | 1.00 | 17.46 | 6 | C |
| ATOM | 103 | CG1 | VAL | A | 13 | −7.651 | 23.946 | 13.381 | 1.00 | 16.11 | 6 | C |
| ATOM | 104 | CG2 | VAL | A | 13 | −5.998 | 22.115 | 13.152 | 1.00 | 16.93 | 6 | C |
| ATOM | 105 | N | THR | A | 14 | −10.290 | 22.532 | 15.313 | 1.00 | 17.07 | 7 | N |
| ATOM | 106 | CA | THR | A | 14 | −11.723 | 22.850 | 15.310 | 1.00 | 18.75 | 6 | C |
| ATOM | 107 | C | THR | A | 14 | −11.977 | 24.057 | 14.402 | 1.00 | 19.10 | 6 | C |
| ATOM | 108 | O | THR | A | 14 | −11.062 | 24.722 | 13.918 | 1.00 | 17.60 | 8 | O |
| ATOM | 109 | CB | THR | A | 14 | −12.269 | 23.193 | 16.704 | 1.00 | 18.82 | 6 | C |
| ATOM | 110 | OG1 | THR | A | 14 | −11.602 | 24.365 | 17.218 | 1.00 | 18.72 | 8 | O |
| ATOM | 111 | CG2 | THR | A | 14 | −12.008 | 22.047 | 17.691 | 1.00 | 19.27 | 6 | C |
| ATOM | 112 | N | GLU | A | 15 | −13.250 | 24.348 | 14.169 | 1.00 | 20.16 | 7 | N |
| ATOM | 113 | CA | GLU | A | 15 | −13.625 | 25.497 | 13.371 | 1.00 | 22.18 | 6 | C |
| ATOM | 114 | C | GLU | A | 15 | −13.077 | 26.825 | 13.926 | 1.00 | 20.98 | 6 | C |
| ATOM | 115 | O | GLU | A | 15 | −12.789 | 26.955 | 15.101 | 1.00 | 19.01 | 8 | O |
| ATOM | 116 | CB | GLU | A | 15 | −15.142 | 25.669 | 13.355 | 1.00 | 25.70 | 6 | C |
| ATOM | 117 | CG | GLU | A | 15 | −15.811 | 24.368 | 12.964 | 1.00 | 29.97 | 6 | C |
| ATOM | 118 | CD | GLU | A | 15 | −16.871 | 24.605 | 11.915 | 1.00 | 32.30 | 6 | C |
| ATOM | 119 | OE1 | GLU | A | 15 | −16.505 | 25.111 | 10.825 | 1.00 | 33.10 | 8 | O |
| ATOM | 120 | OE2 | GLU | A | 15 | −18.032 | 24.282 | 12.228 | 1.00 | 33.52 | 8 | O |
| ATOM | 121 | N | VAL | A | 16 | −13.064 | 27.788 | 13.025 | 1.00 | 20.03 | 7 | N |
| ATOM | 122 | CA | VAL | A | 16 | −12.594 | 29.131 | 13.272 | 1.00 | 18.96 | 6 | C |
| ATOM | 123 | C | VAL | A | 16 | −13.672 | 29.818 | 14.113 | 1.00 | 18.13 | 6 | C |
| ATOM | 124 | O | VAL | A | 16 | −14.869 | 29.675 | 13.882 | 1.00 | 16.52 | 8 | O |
| ATOM | 125 | CB | VAL | A | 16 | −12.332 | 29.865 | 11.954 | 1.00 | 19.26 | 6 | C |
| ATOM | 126 | CG1 | VAL | A | 16 | −12.071 | 31.341 | 12.214 | 1.00 | 21.03 | 6 | C |
| ATOM | 127 | CG2 | VAL | A | 16 | −11.170 | 29.231 | 11.201 | 1.00 | 20.95 | 6 | C |
| ATOM | 128 | N | LYS | A | 17 | −13.194 | 30.499 | 15.135 | 1.00 | 17.20 | 7 | N |
| ATOM | 129 | CA | LYS | A | 17 | −14.022 | 31.172 | 16.106 | 1.00 | 16.66 | 6 | C |
| ATOM | 130 | C | LYS | A | 17 | −13.977 | 32.665 | 15.821 | 1.00 | 18.89 | 6 | C |
| ATOM | 131 | O | LYS | A | 17 | −13.114 | 33.097 | 15.059 | 1.00 | 17.59 | 8 | O |
| ATOM | 132 | CB | LYS | A | 17 | −13.564 | 30.879 | 17.531 | 1.00 | 16.04 | 6 | C |
| ATOM | 133 | CG | LYS | A | 17 | −13.426 | 29.398 | 17.892 | 1.00 | 15.96 | 6 | C |
| ATOM | 134 | CD | LYS | A | 17 | −14.814 | 28.735 | 17.889 | 1.00 | 14.73 | 6 | C |
| ATOM | 135 | CE | LYS | A | 17 | −14.762 | 27.288 | 18.346 | 1.00 | 13.76 | 6 | C |
| ATOM | 136 | NZ | LYS | A | 17 | −13.749 | 26.441 | 17.691 | 1.00 | 12.41 | 7 | N |
| ATOM | 137 | N | TYR | A | 18 | −14.938 | 33.388 | 16.368 | 1.00 | 19.84 | 7 | N |
| ATOM | 138 | CA | TYR | A | 18 | −15.025 | 34.828 | 16.167 | 1.00 | 20.37 | 6 | C |
| ATOM | 139 | C | TYR | A | 18 | −14.923 | 35.541 | 17.504 | 1.00 | 19.90 | 6 | C |
| ATOM | 140 | O | TYR | A | 18 | −15.876 | 35.513 | 18.307 | 1.00 | 17.52 | 8 | O |
| ATOM | 141 | CB | TYR | A | 18 | −16.353 | 35.119 | 15.447 | 1.00 | 21.90 | 6 | C |
| ATOM | 142 | CG | TYR | A | 18 | −16.563 | 36.598 | 15.208 | 1.00 | 24.25 | 6 | C |
| ATOM | 143 | CD1 | TYR | A | 18 | −15.913 | 37.244 | 14.160 | 1.00 | 24.47 | 6 | C |
| ATOM | 144 | CD2 | TYR | A | 18 | −17.397 | 37.334 | 16.040 | 1.00 | 24.72 | 6 | C |
| ATOM | 145 | CE1 | TYR | A | 18 | −16.100 | 38.595 | 13.968 | 1.00 | 26.92 | 6 | C |
| ATOM | 146 | CE2 | TYR | A | 18 | −17.607 | 38.689 | 15.822 | 1.00 | 26.06 | 6 | C |
| ATOM | 147 | CZ | TYR | A | 18 | −16.950 | 39.315 | 14.794 | 1.00 | 26.81 | 6 | C |
| ATOM | 148 | OH | TYR | A | 18 | −17.109 | 40.663 | 14.554 | 1.00 | 28.17 | 8 | O |
| ATOM | 149 | N | GLN | A | 19 | −13.770 | 36.189 | 17.768 | 1.00 | 18.35 | 7 | N |
| ATOM | 150 | CA | GLN | A | 19 | −13.632 | 36.825 | 19.086 | 1.00 | 18.17 | 6 | C |
| ATOM | 151 | C | GLN | A | 19 | −14.517 | 38.055 | 19.257 | 1.00 | 17.45 | 6 | C |
| ATOM | 152 | O | GLN | A | 19 | −14.822 | 38.476 | 20.382 | 1.00 | 16.12 | 8 | O |
| ATOM | 153 | CB | GLN | A | 19 | −12.175 | 37.178 | 19.400 | 1.00 | 17.03 | 6 | C |
| ATOM | 154 | CG | GLN | A | 19 | −11.494 | 38.142 | 18.452 | 1.00 | 17.78 | 6 | C |
| ATOM | 155 | CD | GLN | A | 19 | −9.989 | 38.229 | 18.740 | 1.00 | 18.48 | 6 | C |
| ATOM | 156 | OE1 | GLN | A | 19 | −9.229 | 37.475 | 18.134 | 1.00 | 16.79 | 8 | O |
| ATOM | 157 | NE2 | GLN | A | 19 | −9.561 | 39.125 | 19.629 | 1.00 | 16.10 | 7 | N |
| ATOM | 158 | N | GLY | A | 20 | −14.897 | 38.707 | 18.175 | 1.00 | 17.57 | 7 | N |
| ATOM | 159 | CA | GLY | A | 20 | −15.691 | 39.937 | 18.290 | 1.00 | 19.48 | 6 | C |
| ATOM | 160 | C | GLY | A | 20 | −14.821 | 41.075 | 18.817 | 1.00 | 20.35 | 6 | C |
| ATOM | 161 | O | GLY | A | 20 | −13.586 | 41.062 | 18.656 | 1.00 | 20.41 | 8 | O |
| ATOM | 162 | N | SER | A | 21 | −15.438 | 42.019 | 19.526 | 1.00 | 20.71 | 7 | N |
| ATOM | 163 | CA | SER | A | 21 | −14.713 | 43.160 | 20.063 | 1.00 | 23.14 | 6 | C |
| ATOM | 164 | C | SER | A | 21 | −13.926 | 42.858 | 21.326 | 1.00 | 24.44 | 6 | C |
| ATOM | 165 | O | SER | A | 21 | −12.992 | 43.607 | 21.658 | 1.00 | 26.92 | 8 | O |
| ATOM | 166 | CB | SER | A | 21 | −15.651 | 44.367 | 20.242 | 1.00 | 23.92 | 6 | C |
| ATOM | 167 | OG | SER | A | 21 | −16.759 | 44.065 | 21.045 | 1.00 | 27.49 | 8 | O |
| ATOM | 168 | N | CYS | A | 22 | −14.106 | 41.706 | 21.951 | 1.00 | 22.26 | 7 | N |
| ATOM | 169 | CA | CYS | A | 22 | −13.350 | 41.256 | 23.108 | 1.00 | 21.48 | 6 | C |
| ATOM | 170 | C | CYS | A | 22 | −11.945 | 40.796 | 22.705 | 1.00 | 22.09 | 6 | C |
| ATOM | 171 | O | CYS | A | 22 | −11.829 | 39.925 | 21.840 | 1.00 | 21.19 | 8 | O |
| ATOM | 172 | CB | CYS | A | 22 | −14.080 | 40.099 | 23.784 | 1.00 | 20.91 | 6 | C |
| ATOM | 173 | SG | CYS | A | 22 | −13.219 | 39.203 | 25.087 | 1.00 | 19.30 | 16 | S |
| ATOM | 174 | N | GLY | A | 23 | −10.905 | 41.312 | 23.337 | 1.00 | 22.16 | 7 | N |
| ATOM | 175 | CA | GLY | A | 23 | −9.520 | 40.949 | 23.019 | 1.00 | 23.52 | 6 | C |
| ATOM | 176 | C | GLY | A | 23 | −9.027 | 39.738 | 23.812 | 1.00 | 22.55 | 6 | C |
| ATOM | 177 | O | GLY | A | 23 | −8.098 | 39.854 | 24.600 | 1.00 | 22.31 | 8 | O |
| ATOM | 178 | N | ALA | A | 24 | −9.588 | 38.594 | 23.491 | 1.00 | 20.78 | 7 | N |
| ATOM | 179 | CA | ALA | A | 24 | −9.381 | 37.322 | 24.131 | 1.00 | 22.25 | 6 | C |
| ATOM | 180 | C | ALA | A | 24 | −8.606 | 36.348 | 23.251 | 1.00 | 21.73 | 6 | C |
| ATOM | 181 | O | ALA | A | 24 | −8.604 | 35.133 | 23.477 | 1.00 | 22.17 | 8 | O |
| ATOM | 182 | CB | ALA | A | 24 | −10.770 | 36.696 | 24.361 | 1.00 | 19.41 | 6 | C |
| ATOM | 183 | N | CYS | A | 25 | −8.006 | 36.839 | 22.173 | 1.00 | 21.50 | 7 | N |
| ATOM | 184 | CA | CYS | A | 25 | −7.289 | 35.967 | 21.237 | 1.00 | 21.90 | 6 | C |
| ATOM | 185 | C | CYS | A | 25 | −6.233 | 35.129 | 21.947 | 1.00 | 19.92 | 6 | C |
| ATOM | 186 | O | CYS | A | 25 | −6.046 | 33.974 | 21.548 | 1.00 | 19.41 | 8 | O |
| ATOM | 187 | CB | CYS | A | 25 | −6.710 | 36.813 | 20.107 | 1.00 | 21.33 | 6 | C |
| ATOM | 188 | SG | CYS | A | 25 | −5.315 | 37.751 | 20.742 | 1.00 | 26.09 | 16 | S |
| ATOM | 189 | N | TRP | A | 26 | −5.672 | 35.568 | 23.070 | 1.00 | 17.52 | 7 | N |
| ATOM | 190 | CA | TRP | A | 26 | −4.801 | 34.727 | 23.877 | 1.00 | 18.18 | 6 | C |
| ATOM | 191 | C | TRP | A | 26 | −5.571 | 33.457 | 24.255 | 1.00 | 16.85 | 6 | C |
| ATOM | 192 | O | TRP | A | 26 | −5.008 | 32.382 | 24.189 | 1.00 | 17.48 | 8 | O |
| ATOM | 193 | CB | TRP | A | 26 | −4.313 | 35.435 | 25.137 | 1.00 | 15.96 | 6 | C |
| ATOM | 194 | CG | TRP | A | 26 | −5.404 | 35.886 | 26.061 | 1.00 | 15.80 | 6 | C |
| ATOM | 195 | CD1 | TRP | A | 26 | −6.185 | 37.002 | 25.915 | 1.00 | 15.70 | 6 | C |
| ATOM | 196 | CD2 | TRP | A | 26 | −5.810 | 35.269 | 27.292 | 1.00 | 15.90 | 6 | C |
| ATOM | 197 | NE1 | TRP | A | 26 | −7.063 | 37.094 | 26.970 | 1.00 | 17.03 | 7 | N |
| ATOM | 198 | CE2 | TRP | A | 26 | −6.870 | 36.031 | 27.829 | 1.00 | 15.76 | 6 | C |
| ATOM | 199 | CE3 | TRP | A | 26 | −5.406 | 34.115 | 27.970 | 1.00 | 16.45 | 6 | C |
| ATOM | 200 | CZ2 | TRP | A | 26 | −7.505 | 35.694 | 29.019 | 1.00 | 15.69 | 6 | C |
| ATOM | 201 | CZ3 | TRP | A | 26 | −6.040 | 33.781 | 29.153 | 1.00 | 15.24 | 6 | C |
| ATOM | 202 | CH2 | TRP | A | 26 | −7.079 | 34.570 | 29.665 | 1.00 | 15.78 | 6 | C |
| ATOM | 203 | N | ALA | A | 27 | −6.833 | 33.574 | 24.663 | 1.00 | 16.45 | 7 | N |
| ATOM | 204 | CA | ALA | A | 27 | −7.658 | 32.428 | 25.019 | 1.00 | 16.92 | 6 | C |
| ATOM | 205 | C | ALA | A | 27 | −7.988 | 31.552 | 23.814 | 1.00 | 16.86 | 6 | C |
| ATOM | 206 | O | ALA | A | 27 | −7.857 | 30.334 | 23.908 | 1.00 | 17.19 | 8 | O |
| ATOM | 207 | CB | ALA | A | 27 | −8.960 | 32.917 | 25.669 | 1.00 | 15.94 | 6 | C |
| ATOM | 208 | N | PHE | A | 28 | −8.420 | 32.122 | 22.688 | 1.00 | 15.97 | 7 | N |
| ATOM | 209 | CA | PHE | A | 28 | −8.601 | 31.344 | 21.460 | 1.00 | 18.38 | 6 | C |
| ATOM | 210 | C | PHE | A | 28 | −7.363 | 30.566 | 21.054 | 1.00 | 18.19 | 6 | C |
| ATOM | 211 | O | PHE | A | 28 | −7.370 | 29.370 | 20.699 | 1.00 | 17.48 | 8 | O |
| ATOM | 212 | CB | PHE | A | 28 | −9.138 | 32.265 | 20.342 | 1.00 | 17.61 | 6 | C |
| ATOM | 213 | CG | PHE | A | 28 | −10.587 | 32.608 | 20.629 | 1.00 | 17.23 | 6 | C |
| ATOM | 214 | CD1 | PHE | A | 28 | −10.946 | 33.881 | 21.029 | 1.00 | 17.29 | 6 | C |
| ATOM | 215 | CD2 | PHE | A | 28 | −11.561 | 31.630 | 20.518 | 1.00 | 17.14 | 6 | C |
| ATOM | 216 | CE1 | PHE | A | 28 | −12.261 | 34.188 | 21.307 | 1.00 | 16.44 | 6 | C |
| ATOM | 217 | CE2 | PHE | A | 28 | −12.881 | 31.930 | 20.798 | 1.00 | 16.53 | 6 | C |
| ATOM | 218 | CZ | PHE | A | 28 | −13.223 | 33.210 | 21.187 | 1.00 | 16.26 | 6 | C |
| ATOM | 219 | N | SER | A | 29 | −6.209 | 31.227 | 21.083 | 1.00 | 17.38 | 7 | N |
| ATOM | 220 | CA | SER | A | 29 | −4.923 | 30.616 | 20.795 | 1.00 | 17.21 | 6 | C |
| ATOM | 221 | C | SER | A | 29 | −4.640 | 29.430 | 21.716 | 1.00 | 17.03 | 6 | C |
| ATOM | 222 | O | SER | A | 29 | −4.221 | 28.360 | 21.262 | 1.00 | 16.44 | 8 | O |
| ATOM | 223 | CB | SER | A | 29 | −3.754 | 31.617 | 20.956 | 1.00 | 16.27 | 6 | C |
| ATOM | 224 | OG | SER | A | 29 | −2.523 | 31.035 | 20.620 | 1.00 | 17.21 | 8 | O |
| ATOM | 225 | N | ALA | A | 30 | −4.739 | 29.614 | 23.030 | 1.00 | 16.51 | 7 | N |
| ATOM | 226 | CA | ALA | A | 30 | −4.494 | 28.558 | 24.008 | 1.00 | 16.93 | 6 | C |
| ATOM | 227 | C | ALA | A | 30 | −5.423 | 27.368 | 23.748 | 1.00 | 17.67 | 6 | C |
| ATOM | 228 | O | ALA | A | 30 | −4.965 | 26.220 | 23.590 | 1.00 | 17.18 | 8 | O |
| ATOM | 229 | CB | ALA | A | 30 | −4.774 | 29.082 | 25.419 | 1.00 | 15.31 | 6 | C |
| ATOM | 230 | N | VAL | A | 31 | −6.736 | 27.653 | 23.639 | 1.00 | 15.78 | 7 | N |
| ATOM | 231 | CA | VAL | A | 31 | −7.612 | 26.484 | 23.421 | 1.00 | 16.05 | 6 | C |
| ATOM | 232 | C | VAL | A | 31 | −7.327 | 25.776 | 22.111 | 1.00 | 16.60 | 6 | C |
| ATOM | 233 | O | VAL | A | 31 | −7.375 | 24.537 | 22.106 | 1.00 | 16.65 | 8 | O |
| ATOM | 234 | CB | VAL | A | 31 | −9.115 | 26.767 | 23.633 | 1.00 | 15.32 | 6 | C |
| ATOM | 235 | CG1 | VAL | A | 31 | −9.281 | 27.202 | 25.092 | 1.00 | 13.75 | 6 | C |
| ATOM | 236 | CG2 | VAL | A | 31 | −9.735 | 27.775 | 22.688 | 1.00 | 13.21 | 6 | C |
| ATOM | 237 | N | GLY | A | 32 | −7.020 | 26.454 | 21.018 | 1.00 | 16.40 | 7 | N |
| ATOM | 238 | CA | GLY | A | 32 | −6.681 | 25.786 | 19.771 | 1.00 | 15.93 | 6 | C |
| ATOM | 239 | C | GLY | A | 32 | −5.530 | 24.805 | 19.984 | 1.00 | 15.74 | 6 | C |
| ATOM | 240 | O | GLY | A | 32 | −5.643 | 23.653 | 19.584 | 1.00 | 16.98 | 8 | O |
| ATOM | 241 | N | ALA | A | 33 | −4.470 | 25.226 | 20.645 | 1.00 | 15.94 | 7 | N |
| ATOM | 242 | CA | ALA | A | 33 | −3.327 | 24.336 | 20.882 | 1.00 | 17.89 | 6 | C |
| ATOM | 243 | C | ALA | A | 33 | −3.802 | 23.112 | 21.655 | 1.00 | 17.24 | 6 | C |
| ATOM | 244 | O | ALA | A | 33 | −3.507 | 21.990 | 21.237 | 1.00 | 18.77 | 8 | O |
| ATOM | 245 | CB | ALA | A | 33 | −2.200 | 25.052 | 21.595 | 1.00 | 16.12 | 6 | C |
| ATOM | 246 | N | LEU | A | 34 | −4.576 | 23.329 | 22.719 | 1.00 | 16.31 | 7 | N |
| ATOM | 247 | CA | LEU | A | 34 | −5.024 | 22.215 | 23.542 | 1.00 | 15.76 | 6 | C |
| ATOM | 248 | C | LEU | A | 34 | −6.001 | 21.299 | 22.822 | 1.00 | 16.19 | 6 | C |
| ATOM | 249 | O | LEU | A | 34 | −5.953 | 20.087 | 23.056 | 1.00 | 14.67 | 8 | O |
| ATOM | 250 | CB | LEU | A | 34 | −5.562 | 22.673 | 24.888 | 1.00 | 14.39 | 6 | C |
| ATOM | 251 | CG | LEU | A | 34 | −5.846 | 21.529 | 25.887 | 1.00 | 16.48 | 6 | C |
| ATOM | 252 | CD1 | LEU | A | 34 | −4.628 | 20.643 | 26.138 | 1.00 | 16.05 | 6 | C |
| ATOM | 253 | CD2 | LEU | A | 34 | −6.331 | 22.122 | 27.201 | 1.00 | 14.15 | 6 | C |
| ATOM | 254 | N | GLU | A | 35 | −6.789 | 21.808 | 21.885 | 1.00 | 17.32 | 7 | N |
| ATOM | 255 | CA | GLU | A | 35 | −7.739 | 21.002 | 21.131 | 1.00 | 17.92 | 6 | C |
| ATOM | 256 | C | GLU | A | 35 | −7.053 | 19.951 | 20.273 | 1.00 | 17.29 | 6 | C |
| ATOM | 257 | O | GLU | A | 35 | −7.511 | 18.805 | 20.193 | 1.00 | 16.83 | 8 | O |
| ATOM | 258 | CB | GLU | A | 35 | −8.629 | 21.871 | 20.219 | 1.00 | 17.41 | 6 | C |
| ATOM | 259 | CG | GLU | A | 35 | −9.596 | 22.688 | 21.066 | 1.00 | 17.30 | 6 | C |
| ATOM | 260 | CD | GLU | A | 35 | −10.191 | 23.916 | 20.407 | 1.00 | 17.73 | 6 | C |
| ATOM | 261 | OE1 | GLU | A | 35 | −9.787 | 24.246 | 19.260 | 1.00 | 17.63 | 8 | O |
| ATOM | 262 | OE2 | GLU | A | 35 | −11.011 | 24.593 | 21.050 | 1.00 | 15.27 | 8 | O |
| ATOM | 263 | N | ALA | A | 36 | −5.931 | 20.333 | 19.664 | 1.00 | 17.72 | 7 | N |
| ATOM | 264 | CA | ALA | A | 36 | −5.181 | 19.415 | 18.814 | 1.00 | 18.29 | 6 | C |
| ATOM | 265 | C | ALA | A | 36 | −4.622 | 18.266 | 19.657 | 1.00 | 18.92 | 6 | C |
| ATOM | 266 | O | ALA | A | 36 | −4.705 | 17.097 | 19.307 | 1.00 | 17.72 | 8 | O |
| ATOM | 267 | CB | ALA | A | 36 | −4.011 | 20.082 | 18.116 | 1.00 | 17.84 | 6 | C |
| ATOM | 268 | N | GLN | A | 37 | −4.132 | 18.665 | 20.831 | 1.00 | 19.59 | 7 | N |
| ATOM | 269 | CA | GLN | A | 37 | −3.574 | 17.734 | 21.799 | 1.00 | 19.74 | 6 | C |
| ATOM | 270 | C | GLN | A | 37 | −4.664 | 16.814 | 22.317 | 1.00 | 20.59 | 6 | C |
| ATOM | 271 | O | GLN | A | 37 | −4.479 | 15.597 | 22.406 | 1.00 | 18.18 | 8 | O |
| ATOM | 272 | CB | GLN | A | 37 | −2.869 | 18.473 | 22.932 | 1.00 | 21.11 | 6 | C |
| ATOM | 273 | CG | GLN | A | 37 | −1.571 | 19.185 | 22.571 | 1.00 | 20.44 | 6 | C |
| ATOM | 274 | CD | GLN | A | 37 | −0.609 | 18.366 | 21.748 | 1.00 | 22.15 | 6 | C |
| ATOM | 275 | OE1 | GLN | A | 37 | −0.159 | 17.298 | 22.168 | 1.00 | 21.04 | 8 | O |
| ATOM | 276 | NE2 | GLN | A | 37 | −0.249 | 18.838 | 20.555 | 1.00 | 22.05 | 7 | N |
| ATOM | 277 | N | LEU | A | 38 | −5.892 | 17.335 | 22.554 | 1.00 | 20.72 | 7 | N |
| ATOM | 278 | CA | LEU | A | 38 | −6.948 | 16.463 | 23.026 | 1.00 | 20.79 | 6 | C |
| ATOM | 279 | C | LEU | A | 38 | −7.395 | 15.502 | 21.926 | 1.00 | 22.46 | 6 | C |
| ATOM | 280 | O | LEU | A | 38 | −7.694 | 14.342 | 22.208 | 1.00 | 22.04 | 8 | O |
| ATOM | 281 | CB | LEU | A | 38 | −8.148 | 17.269 | 23.523 | 1.00 | 21.43 | 6 | C |
| ATOM | 282 | CG | LEU | A | 38 | −9.316 | 16.411 | 24.034 | 1.00 | 22.45 | 6 | C |
| ATOM | 283 | CD1 | LEU | A | 38 | −8.946 | 15.693 | 25.333 | 1.00 | 21.59 | 6 | C |
| ATOM | 284 | CD2 | LEU | A | 38 | −10.524 | 17.299 | 24.219 | 1.00 | 22.97 | 6 | C |
| ATOM | 285 | N | LYS | A | 39 | −7.338 | 15.957 | 20.675 | 1.00 | 22.59 | 7 | N |
| ATOM | 286 | CA | LYS | A | 39 | −7.644 | 15.084 | 19.564 | 1.00 | 24.59 | 6 | C |
| ATOM | 287 | C | LYS | A | 39 | −6.621 | 13.949 | 19.511 | 1.00 | 24.30 | 6 | C |
| ATOM | 288 | O | LYS | A | 39 | −7.005 | 12.785 | 19.403 | 1.00 | 23.82 | 8 | O |
| ATOM | 289 | CB | LYS | A | 39 | −7.607 | 15.809 | 18.218 | 1.00 | 24.89 | 6 | C |
| ATOM | 290 | CG | LYS | A | 39 | −8.494 | 15.128 | 17.199 | 1.00 | 26.54 | 6 | C |
| ATOM | 291 | CD | LYS | A | 39 | −7.941 | 13.844 | 16.618 | 1.00 | 26.35 | 6 | C |
| ATOM | 292 | CE | LYS | A | 39 | −8.884 | 13.346 | 15.527 | 1.00 | 27.02 | 6 | C |
| ATOM | 293 | NZ | LYS | A | 39 | −8.087 | 12.728 | 14.424 | 1.00 | 26.16 | 7 | N |
| ATOM | 294 | N | LEU | A | 40 | −5.349 | 14.329 | 19.576 | 1.00 | 24.86 | 7 | N |
| ATOM | 295 | CA | LEU | A | 40 | −4.265 | 13.372 | 19.524 | 1.00 | 26.98 | 6 | C |
| ATOM | 296 | C | LEU | A | 40 | −4.395 | 12.320 | 20.630 | 1.00 | 26.87 | 6 | C |
| ATOM | 297 | O | LEU | A | 40 | −4.152 | 11.168 | 20.318 | 1.00 | 25.13 | 8 | O |
| ATOM | 298 | CB | LEU | A | 40 | −2.867 | 13.994 | 19.607 | 1.00 | 26.48 | 6 | C |
| ATOM | 299 | CG | LEU | A | 40 | −2.094 | 14.431 | 18.367 | 1.00 | 27.33 | 6 | C |
| ATOM | 300 | GD1 | LEU | A | 40 | −2.665 | 13.926 | 17.047 | 1.00 | 26.43 | 6 | C |
| ATOM | 301 | CD2 | LEU | A | 40 | −1.903 | 15.944 | 18.328 | 1.00 | 27.09 | 6 | C |
| ATOM | 302 | N | LYS | A | 41 | −4.876 | 12.649 | 21.826 | 1.00 | 29.03 | 7 | N |
| ATOM | 303 | CA | LYS | A | 41 | −4.899 | 11.684 | 22.912 | 1.00 | 30.09 | 6 | C |
| ATOM | 304 | C | LYS | A | 41 | −6.201 | 10.886 | 22.988 | 1.00 | 30.38 | 6 | C |
| ATOM | 305 | O | LYS | A | 41 | −6.139 | 9.699 | 23.323 | 1.00 | 30.29 | 8 | O |
| ATOM | 306 | CB | LYS | A | 41 | −4.718 | 12.410 | 24.245 | 1.00 | 31.73 | 6 | C |
| ATOM | 307 | CG | LYS | A | 41 | −4.425 | 11.541 | 25.457 | 1.00 | 35.82 | 6 | C |
| ATOM | 308 | CD | LYS | A | 41 | −2.915 | 11.433 | 25.693 | 1.00 | 37.62 | 6 | C |
| ATOM | 309 | CE | LYS | A | 41 | −2.555 | 10.542 | 26.877 | 1.00 | 38.14 | 6 | C |
| ATOM | 310 | NZ | LYS | A | 41 | −2.898 | 11.223 | 28.162 | 1.00 | 39.87 | 7 | N |
| ATOM | 311 | N | THR | A | 42 | −7.339 | 11.513 | 22.725 | 1.00 | 29.06 | 7 | N |
| ATOM | 312 | CA | THR | A | 42 | −8.615 | 10.829 | 22.853 | 1.00 | 29.05 | 6 | C |
| ATOM | 313 | C | THR | A | 42 | −9.263 | 10.346 | 21.564 | 1.00 | 28.87 | 6 | C |
| ATOM | 314 | O | THR | A | 42 | −9.989 | 9.360 | 21.535 | 1.00 | 28.82 | 8 | O |
| ATOM | 315 | CB | THR | A | 42 | −9.644 | 11.751 | 23.549 | 1.00 | 29.64 | 6 | C |
| ATOM | 316 | OG1 | THR | A | 42 | −9.852 | 12.939 | 22.780 | 1.00 | 28.26 | 8 | O |
| ATOM | 317 | CG2 | THR | A | 42 | −9.133 | 12.094 | 24.941 | 1.00 | 30.34 | 6 | C |
| ATOM | 318 | N | GLY | A | 43 | −8.885 | 10.983 | 20.460 | 1.00 | 29.52 | 7 | N |
| ATOM | 319 | CA | GLY | A | 43 | −9.440 | 10.699 | 19.142 | 1.00 | 28.57 | 6 | C |
| ATOM | 320 | C | GLY | A | 43 | −10.634 | 11.621 | 18.886 | 1.00 | 28.49 | 6 | C |
| ATOM | 321 | O | GLY | A | 43 | −11.313 | 11.670 | 17.846 | 1.00 | 28.02 | 8 | O |
| ATOM | 322 | N | LYS | A | 44 | −10.936 | 12.456 | 19.888 | 1.00 | 27.55 | 7 | N |
| ATOM | 323 | CA | LYS | A | 44 | −12.055 | 13.368 | 19.776 | 1.00 | 28.20 | 6 | C |
| ATOM | 324 | C | LYS | A | 44 | −11.651 | 14.827 | 19.551 | 1.00 | 26.36 | 6 | C |
| ATOM | 325 | O | LYS | A | 44 | −10.843 | 15.430 | 20.242 | 1.00 | 24.53 | 8 | O |
| ATOM | 326 | CB | LYS | A | 44 | −13.025 | 13.392 | 20.952 | 1.00 | 30.93 | 6 | C |
| ATOM | 327 | CG | LYS | A | 44 | −13.525 | 12.083 | 21.501 | 1.00 | 35.12 | 6 | C |
| ATOM | 328 | CD | LYS | A | 44 | −14.483 | 12.316 | 22.659 | 1.00 | 37.99 | 6 | C |
| ATOM | 329 | CE | LYS | A | 44 | −15.887 | 12.738 | 22.285 | 1.00 | 40.19 | 6 | C |
| ATOM | 330 | NZ | LYS | A | 44 | −16.222 | 14.171 | 22.486 | 1.00 | 40.87 | 7 | N |
| ATOM | 331 | N | LEU | A | 45 | −12.328 | 15.396 | 18.569 | 1.00 | 23.85 | 7 | N |
| ATOM | 332 | CA | LEU | A | 45 | −12.197 | 16.771 | 18.159 | 1.00 | 23.20 | 6 | C |
| ATOM | 333 | C | LEU | A | 45 | −13.348 | 17.560 | 18.779 | 1.00 | 23.59 | 6 | C |
| ATOM | 334 | O | LEU | A | 45 | −14.505 | 17.398 | 18.406 | 1.00 | 24.24 | 8 | O |
| ATOM | 335 | CB | LEU | A | 45 | −12.289 | 16.971 | 16.642 | 1.00 | 21.33 | 6 | C |
| ATOM | 336 | CG | LEU | A | 45 | −11.996 | 18.414 | 16.193 | 1.00 | 20.74 | 6 | C |
| ATOM | 337 | CD1 | LEU | A | 45 | −10.622 | 18.872 | 16.656 | 1.00 | 19.65 | 6 | C |
| ATOM | 338 | CD2 | LEU | A | 45 | −12.039 | 18.494 | 14.669 | 1.00 | 20.45 | 6 | C |
| ATOM | 339 | N | VAL | A | 46 | −13.035 | 18.364 | 19.777 | 1.00 | 24.57 | 7 | N |
| ATOM | 340 | CA | VAL | A | 46 | −13.976 | 19.138 | 20.542 | 1.00 | 24.31 | 6 | C |
| ATOM | 341 | C | VAL | A | 46 | −13.466 | 20.551 | 20.817 | 1.00 | 22.73 | 6 | C |
| ATOM | 342 | O | VAL | A | 46 | −12.279 | 20.726 | 21.154 | 1.00 | 22.54 | 8 | O |
| ATOM | 343 | CB | VAL | A | 46 | −14.366 | 18.514 | 21.895 | 1.00 | 26.41 | 6 | C |
| ATOM | 344 | CG1 | VAL | A | 46 | −14.971 | 17.116 | 21.771 | 1.00 | 27.91 | 6 | C |
| ATOM | 345 | CG2 | VAL | A | 46 | −13.286 | 18.454 | 22.944 | 1.00 | 28.91 | 6 | C |
| ATOM | 346 | N | SER | A | 47 | −14.317 | 21.545 | 20.624 | 1.00 | 19.05 | 7 | N |
| ATOM | 347 | CA | SER | A | 47 | −13.960 | 22.936 | 20.860 | 1.00 | 18.99 | 6 | C |
| ATOM | 348 | C | SER | A | 47 | −13.947 | 23.245 | 22.359 | 1.00 | 18.85 | 6 | C |
| ATOM | 349 | O | SER | A | 47 | −14.930 | 23.066 | 23.069 | 1.00 | 18.08 | 8 | O |
| ATOM | 350 | CB | SER | A | 47 | −14.866 | 23.942 | 20.158 | 1.00 | 18.32 | 6 | C |
| ATOM | 351 | OG | SER | A | 47 | −14.740 | 23.941 | 18.751 | 1.00 | 17.10 | 8 | O |
| ATOM | 352 | N | LEU | A | 48 | −12.786 | 23.730 | 22.830 | 1.00 | 17.49 | 7 | N |
| ATOM | 353 | CA | LEU | A | 48 | −12.677 | 24.039 | 24.254 | 1.00 | 16.34 | 6 | C |
| ATOM | 354 | C | LEU | A | 48 | −13.155 | 25.427 | 24.628 | 1.00 | 17.70 | 6 | C |
| ATOM | 355 | O | LEU | A | 48 | −13.196 | 26.338 | 23.792 | 1.00 | 16.84 | 8 | O |
| ATOM | 356 | CB | LEU | A | 48 | −11.232 | 23.779 | 24.712 | 1.00 | 17.83 | 6 | C |
| ATOM | 357 | CG | LEU | A | 48 | −10.824 | 22.318 | 24.448 | 1.00 | 18.14 | 6 | C |
| ATOM | 358 | CD1 | LEU | A | 48 | −9.342 | 22.056 | 24.683 | 1.00 | 17.67 | 6 | C |
| ATOM | 359 | CD2 | LEU | A | 48 | −11.669 | 21.439 | 25.392 | 1.00 | 18.70 | 6 | C |
| ATOM | 360 | N | SER | A | 49 | −13.433 | 25.653 | 25.918 | 1.00 | 15.90 | 7 | N |
| ATOM | 361 | CA | SER | A | 49 | −13.933 | 26.933 | 26.369 | 1.00 | 18.00 | 6 | C |
| ATOM | 362 | C | SER | A | 49 | −12.896 | 28.046 | 26.478 | 1.00 | 17.99 | 6 | C |
| ATOM | 363 | O | SER | A | 49 | −12.189 | 28.151 | 27.474 | 1.00 | 17.35 | 8 | O |
| ATOM | 364 | CB | SER | A | 49 | −14.623 | 26.778 | 27.746 | 1.00 | 18.02 | 6 | C |
| ATOM | 365 | OG | SER | A | 49 | −15.129 | 28.046 | 28.144 | 1.00 | 18.86 | 8 | O |
| ATOM | 366 | N | ALA | A | 50 | −12.960 | 29.021 | 25.570 | 1.00 | 18.67 | 7 | N |
| ATOM | 367 | CA | ALA | A | 50 | −12.153 | 30.224 | 25.629 | 1.00 | 18.60 | 6 | C |
| ATOM | 368 | C | ALA | A | 50 | −12.619 | 31.174 | 26.724 | 1.00 | 17.87 | 6 | C |
| ATOM | 369 | O | ALA | A | 50 | −11.815 | 31.901 | 27.322 | 1.00 | 16.25 | 8 | O |
| ATOM | 370 | CB | ALA | A | 50 | −12.123 | 30.981 | 24.300 | 1.00 | 11.21 | 6 | C |
| ATOM | 371 | N | GLN | A | 51 | −13.922 | 31.177 | 26.988 | 1.00 | 18.60 | 7 | N |
| ATOM | 372 | CA | GLN | A | 51 | −14.542 | 31.983 | 28.024 | 1.00 | 18.40 | 6 | C |
| ATOM | 373 | C | GLN | A | 51 | −14.076 | 31.556 | 29.411 | 1.00 | 18.48 | 6 | C |
| ATOM | 374 | O | GLN | A | 51 | −13.943 | 32.321 | 30.358 | 1.00 | 18.80 | 8 | O |
| ATOM | 375 | CB | GLN | A | 51 | −16.080 | 31.808 | 27.962 | 1.00 | 20.13 | 6 | C |
| ATOM | 376 | CG | GLN | A | 51 | −16.783 | 32.937 | 28.702 | 1.00 | 18.55 | 6 | C |
| ATOM | 377 | CD | GLN | A | 51 | −16.661 | 34.326 | 28.120 | 1.00 | 17.07 | 6 | C |
| ATOM | 378 | OE1 | GLN | A | 51 | −16.824 | 34.535 | 26.923 | 1.00 | 14.91 | 8 | O |
| ATOM | 379 | NE2 | GLN | A | 51 | −16.460 | 35.288 | 29.011 | 1.00 | 18.51 | 7 | N |
| ATOM | 380 | N | ASN | A | 52 | −13.963 | 30.225 | 29.575 | 1.00 | 19.47 | 7 | N |
| ATOM | 381 | CA | ASN | A | 52 | −13.460 | 29.631 | 30.815 | 1.00 | 20.71 | 6 | C |
| ATOM | 382 | C | ASN | A | 52 | −12.124 | 30.280 | 31.181 | 1.00 | 20.45 | 6 | C |
| ATOM | 383 | O | ASN | A | 52 | −11.944 | 30.762 | 32.303 | 1.00 | 17.25 | 8 | O |
| ATOM | 384 | CB | ASN | A | 52 | −13.383 | 28.114 | 30.673 | 1.00 | 20.87 | 6 | C |
| ATOM | 385 | CG | ASN | A | 52 | −12.974 | 27.281 | 31.848 | 1.00 | 20.23 | 6 | C |
| ATOM | 386 | OD1 | ASN | A | 52 | −13.035 | 26.042 | 31.784 | 1.00 | 20.32 | 8 | O |
| ATOM | 387 | ND2 | ASN | A | 52 | −12.517 | 27.875 | 32.946 | 1.00 | 21.50 | 7 | N |
| ATOM | 388 | N | LEU | A | 53 | −11.198 | 30.420 | 30.215 | 1.00 | 21.47 | 7 | N |
| ATOM | 389 | CA | LEU | A | 53 | −9.912 | 31.043 | 30.494 | 1.00 | 21.14 | 6 | C |
| ATOM | 390 | C | LEU | A | 53 | −10.107 | 32.536 | 30.727 | 1.00 | 21.35 | 6 | C |
| ATOM | 391 | O | LEU | A | 53 | −9.471 | 33.134 | 31.587 | 1.00 | 18.78 | 8 | O |
| ATOM | 392 | CB | LEU | A | 53 | −8.921 | 30.819 | 29.359 | 1.00 | 21.56 | 6 | C |
| ATOM | 393 | CG | LEU | A | 53 | −8.456 | 29.377 | 29.114 | 1.00 | 20.07 | 6 | C |
| ATOM | 394 | CD1 | LEU | A | 53 | −7.771 | 29.374 | 27.757 | 1.00 | 19.70 | 6 | C |
| ATOM | 395 | CD2 | LEU | A | 53 | −7.599 | 28.877 | 30.261 | 1.00 | 18.58 | 6 | C |
| ATOM | 396 | N | VAL | A | 54 | −10.993 | 33.152 | 29.946 | 1.00 | 22.68 | 7 | N |
| ATOM | 397 | CA | VAL | A | 54 | −11.254 | 34.588 | 30.057 | 1.00 | 23.62 | 6 | C |
| ATOM | 398 | C | VAL | A | 54 | −11.728 | 34.982 | 31.448 | 1.00 | 24.36 | 6 | C |
| ATOM | 399 | O | VAL | A | 54 | −11.226 | 35.919 | 32.085 | 1.00 | 23.55 | 8 | O |
| ATOM | 400 | CB | VAL | A | 54 | −12.248 | 35.049 | 28.978 | 1.00 | 23.79 | 6 | C |
| ATOM | 401 | CG1 | VAL | A | 54 | −12.803 | 36.440 | 29.246 | 1.00 | 24.54 | 6 | C |
| ATOM | 402 | CG2 | VAL | A | 54 | −11.545 | 35.029 | 27.619 | 1.00 | 24.34 | 6 | C |
| ATOM | 403 | N | ASP | A | 55 | −12.688 | 34.213 | 31.951 | 1.00 | 26.26 | 7 | N |
| ATOM | 404 | CA | ASP | A | 55 | −13.231 | 34.456 | 33.279 | 1.00 | 26.05 | 6 | C |
| ATOM | 405 | C | ASP | A | 55 | −12.428 | 33.847 | 34.427 | 1.00 | 25.32 | 6 | C |
| ATOM | 406 | O | ASP | A | 55 | −12.645 | 34.315 | 35.554 | 1.00 | 25.80 | 8 | O |
| ATOM | 407 | CB | ASP | A | 55 | −14.600 | 33.775 | 33.426 | 1.00 | 25.71 | 6 | C |
| ATOM | 408 | CG | ASP | A | 55 | −15.603 | 34.072 | 32.344 | 1.00 | 25.74 | 6 | C |
| ATOM | 409 | OD1 | ASP | A | 55 | −15.467 | 35.111 | 31.668 | 1.00 | 24.78 | 8 | O |
| ATOM | 410 | OD2 | ASP | A | 55 | −16.507 | 33.210 | 32.219 | 1.00 | 24.52 | 8 | O |
| ATOM | 411 | N | CYS | A | 56 | −11.662 | 32.793 | 34.201 | 1.00 | 23.81 | 7 | N |
| ATOM | 412 | CA | CYS | A | 56 | −11.012 | 32.164 | 35.354 | 1.00 | 23.78 | 6 | C |
| ATOM | 413 | C | CYS | A | 56 | −9.501 | 32.282 | 35.408 | 1.00 | 23.35 | 6 | C |
| ATOM | 414 | O | CYS | A | 56 | −8.918 | 32.128 | 36.477 | 1.00 | 21.47 | 8 | O |
| ATOM | 415 | CB | CYS | A | 56 | −11.414 | 30.683 | 35.404 | 1.00 | 23.68 | 6 | C |
| ATOM | 416 | SG | CYS | A | 56 | −13.190 | 30.368 | 35.221 | 1.00 | 22.70 | 16 | S |
| ATOM | 417 | N | SER | A | 57 | −8.835 | 32.493 | 34.281 | 1.00 | 22.84 | 7 | N |
| ATOM | 418 | CA | SER | A | 57 | −7.366 | 32.608 | 34.322 | 1.00 | 23.73 | 6 | C |
| ATOM | 419 | C | SER | A | 57 | −7.071 | 34.101 | 34.398 | 1.00 | 24.63 | 6 | C |
| ATOM | 420 | O | SER | A | 57 | −6.849 | 34.727 | 33.367 | 1.00 | 26.03 | 8 | O |
| ATOM | 421 | CB | SER | A | 57 | −6.793 | 31.920 | 33.096 | 1.00 | 21.04 | 6 | C |
| ATOM | 422 | OG | SER | A | 57 | −5.422 | 32.154 | 32.935 | 1.00 | 19.66 | 8 | O |
| ATOM | 423 | N | THR | A | 58 | −7.302 | 34.714 | 35.549 | 1.00 | 26.44 | 7 | N |
| ATOM | 424 | CA | THR | A | 58 | −7.254 | 36.172 | 35.613 | 1.00 | 28.22 | 6 | C |
| ATOM | 425 | C | THR | A | 58 | −5.961 | 36.646 | 36.248 | 1.00 | 28.24 | 6 | C |
| ATOM | 426 | O | THR | A | 58 | −4.871 | 36.239 | 35.841 | 1.00 | 27.63 | 8 | O |
| ATOM | 427 | CB | THR | A | 58 | −8.524 | 36.736 | 36.266 | 1.00 | 28.11 | 6 | C |
| ATOM | 428 | OG1 | THR | A | 58 | −8.677 | 36.209 | 37.582 | 1.00 | 28.90 | 8 | O |
| ATOM | 429 | CG2 | THR | A | 58 | −9.756 | 36.367 | 35.448 | 1.00 | 28.10 | 6 | C |
| ATOM | 430 | N | GLU | A | 59 | −6.047 | 37.550 | 37.200 | 1.00 | 29.07 | 7 | N |
| ATOM | 431 | CA | GLU | A | 59 | −4.971 | 38.180 | 37.914 | 1.00 | 29.69 | 6 | C |
| ATOM | 432 | C | GLU | A | 59 | −3.748 | 37.343 | 38.226 | 1.00 | 29.57 | 6 | C |
| ATOM | 433 | O | GLU | A | 59 | −2.616 | 37.789 | 38.002 | 1.00 | 27.85 | 8 | O |
| ATOM | 434 | CB | GLU | A | 59 | −5.480 | 38.705 | 39.283 | 1.00 | 30.22 | 6 | C |
| ATOM | 435 | CG | GLU | A | 59 | −4.525 | 39.351 | 40.197 | 1.00 | 20.00 | 6 | C |
| ATOM | 436 | CD | GLU | A | 59 | −5.160 | 39.754 | 41.508 | 1.00 | 20.00 | 6 | C |
| ATOM | 437 | OE1 | GLU | A | 59 | −4.490 | 40.384 | 42.314 | 1.00 | 20.00 | 8 | O |
| ATOM | 438 | OE2 | GLU | A | 59 | −6.328 | 39.431 | 41.715 | 1.00 | 20.00 | 8 | O |
| ATOM | 439 | N | LYS | A | 60 | −3.927 | 36.137 | 38.777 | 1.00 | 30.46 | 7 | N |
| ATOM | 440 | CA | LYS | A | 60 | −2.794 | 35.303 | 39.152 | 1.00 | 30.95 | 6 | C |
| ATOM | 441 | C | LYS | A | 60 | −1.904 | 34.881 | 37.995 | 1.00 | 28.55 | 6 | C |
| ATOM | 442 | O | LYS | A | 60 | −0.785 | 34.468 | 38.236 | 1.00 | 28.35 | 8 | O |
| ATOM | 443 | CB | LYS | A | 60 | −3.242 | 33.999 | 39.830 | 1.00 | 31.93 | 6 | C |
| ATOM | 444 | CG | LYS | A | 60 | −4.331 | 34.139 | 40.872 | 1.00 | 33.59 | 6 | C |
| ATOM | 445 | CD | LYS | A | 60 | −4.246 | 33.139 | 41.909 | 1.00 | 47.84 | 6 | C |
| ATOM | 446 | CE | LYS | A | 60 | −4.061 | 31.694 | 41.491 | 1.00 | 49.56 | 6 | C |
| ATOM | 447 | NZ | LYS | A | 60 | −3.084 | 30.880 | 42.263 | 1.00 | 51.15 | 7 | N |
| ATOM | 448 | N | TYR | A | 61 | −2.395 | 34.911 | 36.768 | 1.00 | 28.73 | 7 | N |
| ATOM | 449 | CA | TYR | A | 61 | −1.638 | 34.493 | 35.586 | 1.00 | 27.32 | 6 | C |
| ATOM | 450 | C | TYR | A | 61 | −1.289 | 35.697 | 34.729 | 1.00 | 25.61 | 6 | C |
| ATOM | 451 | O | TYR | A | 61 | −1.163 | 35.568 | 33.507 | 1.00 | 26.14 | 8 | O |
| ATOM | 452 | CB | TYR | A | 61 | −2.531 | 33.479 | 34.848 | 1.00 | 26.85 | 6 | C |
| ATOM | 453 | CG | TYR | A | 61 | −2.893 | 32.299 | 35.734 | 1.00 | 27.97 | 6 | C |
| ATOM | 454 | CD1 | TYR | A | 61 | −4.083 | 32.271 | 36.447 | 1.00 | 28.40 | 6 | C |
| ATOM | 455 | CD2 | TYR | A | 61 | −2.026 | 31.230 | 35.885 | 1.00 | 27.30 | 6 | C |
| ATOM | 456 | CE1 | TYR | A | 61 | −4.410 | 31.209 | 37.276 | 1.00 | 29.07 | 6 | C |
| ATOM | 457 | CE2 | TYR | A | 61 | −2.323 | 30.162 | 36.696 | 1.00 | 27.86 | 6 | C |
| ATOM | 458 | CZ | TYR | A | 61 | −3.521 | 30.151 | 37.383 | 1.00 | 29.19 | 6 | C |
| ATOM | 459 | OH | TYR | A | 61 | −3.797 | 29.082 | 38.191 | 1.00 | 28.61 | 8 | O |
| ATOM | 460 | N | GLY | A | 62 | −1.544 | 36.887 | 35.258 | 1.00 | 24.11 | 7 | N |
| ATOM | 461 | CA | GLY | A | 62 | −1.322 | 38.145 | 34.567 | 1.00 | 24.11 | 6 | C |
| ATOM | 462 | C | GLY | A | 62 | −2.211 | 38.377 | 33.357 | 1.00 | 24.84 | 6 | C |
| ATOM | 463 | O | GLY | A | 62 | −1.857 | 39.169 | 32.478 | 1.00 | 25.32 | 8 | O |
| ATOM | 464 | N | ASN | A | 63 | −3.347 | 37.711 | 33.279 | 1.00 | 24.05 | 7 | N |
| ATOM | 465 | CA | ASN | A | 63 | −4.299 | 37.817 | 32.191 | 1.00 | 25.04 | 6 | C |
| ATOM | 466 | C | ASN | A | 63 | −5.419 | 38.790 | 32.560 | 1.00 | 25.68 | 6 | C |
| ATOM | 467 | O | ASN | A | 63 | −5.860 | 38.832 | 33.705 | 1.00 | 25.85 | 8 | O |
| ATOM | 468 | CB | ASN | A | 63 | −4.897 | 36.438 | 31.865 | 1.00 | 24.60 | 6 | C |
| ATOM | 469 | CG | ASN | A | 63 | −3.790 | 35.499 | 31.409 | 1.00 | 25.30 | 6 | C |
| ATOM | 470 | OD1 | ASN | A | 63 | −2.863 | 36.025 | 30.785 | 1.00 | 23.98 | 8 | O |
| ATOM | 471 | ND2 | ASN | A | 63 | −3.881 | 34.212 | 31.716 | 1.00 | 23.73 | 7 | N |
| ATOM | 472 | N | LYS | A | 64 | −5.848 | 39.591 | 31.600 | 1.00 | 26.83 | 7 | N |
| ATOM | 473 | CA | LYS | A | 64 | −6.905 | 40.557 | 31.862 | 1.00 | 29.03 | 6 | C |
| ATOM | 474 | C | LYS | A | 64 | −8.145 | 40.425 | 30.994 | 1.00 | 26.94 | 6 | C |
| ATOM | 475 | O | LYS | A | 64 | −8.688 | 41.414 | 30.515 | 1.00 | 23.94 | 8 | O |
| ATOM | 476 | CB | LYS | A | 64 | −6.366 | 41.986 | 31.799 | 1.00 | 31.45 | 6 | C |
| ATOM | 477 | CG | LYS | A | 64 | −4.951 | 42.150 | 31.300 | 1.00 | 34.48 | 6 | C |
| ATOM | 478 | CD | LYS | A | 64 | −4.374 | 43.468 | 31.796 | 1.00 | 36.59 | 6 | C |
| ATOM | 479 | CE | LYS | A | 64 | −3.941 | 43.380 | 33.247 | 1.00 | 38.18 | 6 | C |
| ATOM | 480 | NZ | LYS | A | 64 | −3.789 | 44.751 | 33.836 | 1.00 | 39.69 | 7 | N |
| ATOM | 481 | N | GLY | A | 65 | −8.652 | 39.205 | 30.874 | 1.00 | 26.14 | 7 | N |
| ATOM | 482 | CA | GLY | A | 65 | −9.905 | 38.924 | 30.188 | 1.00 | 25.32 | 6 | C |
| ATOM | 483 | C | GLY | A | 65 | −9.950 | 39.464 | 28.776 | 1.00 | 25.36 | 6 | C |
| ATOM | 484 | O | GLY | A | 65 | −9.088 | 39.160 | 27.950 | 1.00 | 23.67 | 8 | O |
| ATOM | 485 | N | CYS | A | 66 | −10.930 | 40.328 | 28.492 | 1.00 | 23.74 | 7 | N |
| ATOM | 486 | CA | CYS | A | 66 | −11.081 | 40.926 | 27.176 | 1.00 | 23.88 | 6 | C |
| ATOM | 487 | C | CYS | A | 66 | −10.051 | 42.024 | 26.939 | 1.00 | 25.35 | 6 | C |
| ATOM | 488 | O | CYS | A | 66 | −9.951 | 42.541 | 25.819 | 1.00 | 27.33 | 8 | O |
| ATOM | 489 | CB | CYS | A | 66 | −12.485 | 41.490 | 26.912 | 1.00 | 22.99 | 6 | C |
| ATOM | 490 | SG | CYS | A | 66 | −13.782 | 40.217 | 26.798 | 1.00 | 20.78 | 16 | S |
| ATOM | 491 | N | ASN | A | 67 | −9.190 | 42.325 | 27.902 | 1.00 | 25.39 | 7 | N |
| ATOM | 492 | CA | ASN | A | 67 | −8.101 | 43.261 | 27.688 | 1.00 | 24.98 | 6 | C |
| ATOM | 493 | C | ASN | A | 67 | −6.780 | 42.534 | 27.449 | 1.00 | 23.86 | 6 | C |
| ATOM | 494 | O | ASN | A | 67 | −5.720 | 43.151 | 27.562 | 1.00 | 22.02 | 8 | O |
| ATOM | 495 | CB | ASN | A | 67 | −8.037 | 44.271 | 28.831 | 1.00 | 26.60 | 6 | C |
| ATOM | 496 | CG | ASN | A | 67 | −9.185 | 45.261 | 28.730 | 1.00 | 29.73 | 6 | C |
| ATOM | 497 | OD1 | ASN | A | 67 | −9.581 | 45.672 | 27.636 | 1.00 | 31.40 | 8 | O |
| ATOM | 498 | ND2 | ASN | A | 67 | −9.724 | 45.670 | 29.869 | 1.00 | 30.23 | 7 | N |
| ATOM | 499 | N | GLY | A | 68 | −6.803 | 41.249 | 27.099 | 1.00 | 22.75 | 7 | N |
| ATOM | 500 | CA | GLY | A | 68 | −5.543 | 40.589 | 26.755 | 1.00 | 23.82 | 6 | C |
| ATOM | 501 | C | GLY | A | 68 | −5.019 | 39.668 | 27.841 | 1.00 | 24.11 | 6 | C |
| ATOM | 502 | O | GLY | A | 68 | −5.545 | 39.564 | 28.948 | 1.00 | 25.80 | 8 | O |
| ATOM | 503 | N | GLY | A | 69 | −3.988 | 38.908 | 27.446 | 1.00 | 23.67 | 7 | N |
| ATOM | 504 | CA | GLY | A | 69 | −3.415 | 37.927 | 28.370 | 1.00 | 23.59 | 6 | C |
| ATOM | 505 | C | GLY | A | 69 | −2.368 | 37.026 | 27.735 | 1.00 | 22.71 | 6 | C |
| ATOM | 506 | O | GLY | A | 69 | −1.880 | 37.295 | 26.627 | 1.00 | 23.06 | 8 | O |
| ATOM | 507 | N | PHE | A | 70 | −2.040 | 35.925 | 28.417 | 1.00 | 21.84 | 7 | N |
| ATOM | 508 | CA | PHE | A | 70 | −0.985 | 35.045 | 27.942 | 1.00 | 20.02 | 6 | C |
| ATOM | 509 | C | PHE | A | 70 | −1.441 | 33.614 | 27.673 | 1.00 | 19.58 | 6 | C |
| ATOM | 510 | O | PHE | A | 70 | −2.020 | 32.987 | 28.565 | 1.00 | 18.51 | 8 | O |
| ATOM | 511 | CB | PHE | A | 70 | 0.120 | 34.982 | 29.009 | 1.00 | 21.03 | 6 | C |
| ATOM | 512 | CG | PHE | A | 70 | 0.686 | 36.311 | 29.430 | 1.00 | 22.23 | 6 | C |
| ATOM | 513 | CD1 | PHE | A | 70 | 0.317 | 36.909 | 30.617 | 1.00 | 23.39 | 6 | C |
| ATOM | 514 | CD2 | PHE | A | 70 | 1.566 | 36.983 | 28.616 | 1.00 | 22.82 | 6 | C |
| ATOM | 515 | CE1 | PHE | A | 70 | 0.843 | 38.125 | 31.005 | 1.00 | 23.39 | 6 | C |
| ATOM | 516 | CE2 | PHE | A | 70 | 2.095 | 38.227 | 28.996 | 1.00 | 24.65 | 6 | C |
| ATOM | 517 | CZ | PHE | A | 70 | 1.742 | 38.795 | 30.210 | 1.00 | 23.07 | 6 | C |
| ATOM | 518 | N | MET | A | 71 | −0.991 | 33.056 | 26.541 | 1.00 | 18.47 | 7 | N |
| ATOM | 519 | CA | MET | A | 71 | −1.259 | 31.630 | 26.295 | 1.00 | 18.99 | 6 | C |
| ATOM | 520 | C | MET | A | 71 | −0.529 | 30.728 | 27.301 | 1.00 | 18.18 | 6 | C |
| ATOM | 521 | O | MET | A | 71 | −1.071 | 29.694 | 27.712 | 1.00 | 17.50 | 8 | O |
| ATOM | 522 | CB | MET | A | 71 | −0.922 | 31.207 | 24.871 | 1.00 | 18.74 | 6 | C |
| ATOM | 523 | CG | MET | A | 71 | −1.791 | 31.818 | 23.776 | 1.00 | 18.37 | 6 | C |
| ATOM | 524 | SD | MET | A | 71 | −1.216 | 33.440 | 23.244 | 1.00 | 18.63 | 16 | S |
| ATOM | 525 | CE | MET | A | 71 | 0.343 | 32.988 | 22.505 | 1.00 | 18.77 | 6 | C |
| ATOM | 526 | N | THR | A | 72 | 0.727 | 31.021 | 27.622 | 1.00 | 18.05 | 7 | N |
| ATOM | 527 | CA | THR | A | 72 | 1.471 | 30.174 | 28.548 | 1.00 | 20.31 | 6 | C |
| ATOM | 528 | C | THR | A | 72 | 0.785 | 30.118 | 29.911 | 1.00 | 20.07 | 6 | C |
| ATOM | 529 | O | THR | A | 72 | 0.622 | 29.054 | 30.503 | 1.00 | 19.51 | 8 | O |
| ATOM | 530 | CB | THR | A | 72 | 2.930 | 30.597 | 28.743 | 1.00 | 21.33 | 6 | C |
| ATOM | 531 | OG1 | THR | A | 72 | 2.975 | 31.999 | 29.079 | 1.00 | 22.82 | 8 | O |
| ATOM | 532 | CG2 | THR | A | 72 | 3.831 | 30.406 | 27.534 | 1.00 | 21.24 | 6 | C |
| ATOM | 533 | N | THR | A | 73 | 0.357 | 31.257 | 30.457 | 1.00 | 21.16 | 7 | N |
| ATOM | 534 | CA | THR | A | 73 | −0.286 | 31.292 | 31.764 | 1.00 | 22.38 | 6 | C |
| ATOM | 535 | C | THR | A | 73 | −1.721 | 30.785 | 31.692 | 1.00 | 22.86 | 6 | C |
| ATOM | 536 | O | THR | A | 73 | −2.248 | 30.214 | 32.649 | 1.00 | 22.70 | 8 | O |
| ATOM | 537 | CB | THR | A | 73 | −0.263 | 32.670 | 32.435 | 1.00 | 23.50 | 6 | C |
| ATOM | 538 | OG1 | THR | A | 73 | −1.106 | 33.563 | 31.698 | 1.00 | 23.93 | 8 | O |
| ATOM | 539 | CG2 | THR | A | 73 | 1.157 | 33.227 | 32.500 | 1.00 | 23.17 | 6 | C |
| ATOM | 540 | N | ALA | A | 74 | −2.321 | 30.888 | 30.503 | 1.00 | 21.66 | 7 | N |
| ATOM | 541 | CA | ALA | A | 74 | −3.598 | 30.218 | 30.260 | 1.00 | 21.26 | 6 | C |
| ATOM | 542 | C | ALA | A | 74 | −3.391 | 28.717 | 30.492 | 1.00 | 20.84 | 6 | C |
| ATOM | 543 | O | ALA | A | 74 | −4.157 | 28.084 | 31.222 | 1.00 | 19.57 | 8 | O |
| ATOM | 544 | CB | ALA | A | 74 | −4.100 | 30.489 | 28.858 | 1.00 | 20.60 | 6 | C |
| ATOM | 545 | N | PHE | A | 75 | −2.299 | 28.158 | 29.955 | 1.00 | 20.34 | 7 | N |
| ATOM | 546 | CA | PHE | A | 75 | −2.044 | 26.730 | 30.158 | 1.00 | 21.42 | 6 | C |
| ATOM | 547 | C | PHE | A | 75 | −1.813 | 26.393 | 31.634 | 1.00 | 22.13 | 6 | C |
| ATOM | 548 | O | PHE | A | 75 | −2.331 | 25.360 | 32.110 | 1.00 | 21.75 | 8 | O |
| ATOM | 549 | CB | PHE | A | 75 | −0.900 | 26.222 | 29.292 | 1.00 | 19.45 | 6 | C |
| ATOM | 550 | CG | PHE | A | 75 | −1.024 | 26.378 | 27.801 | 1.00 | 19.54 | 6 | C |
| ATOM | 551 | CD1 | PHE | A | 75 | −2.197 | 26.063 | 27.132 | 1.00 | 17.93 | 6 | C |
| ATOM | 552 | CD2 | PHE | A | 75 | 0.040 | 26.884 | 27.073 | 1.00 | 17.78 | 6 | C |
| ATOM | 553 | CE1 | PHE | A | 75 | −2.291 | 26.192 | 25.760 | 1.00 | 17.68 | 6 | C |
| ATOM | 554 | CE2 | PHE | A | 75 | −0.063 | 27.033 | 25.702 | 1.00 | 18.90 | 6 | C |
| ATOM | 555 | CZ | PHE | A | 75 | −1.242 | 26.707 | 25.039 | 1.00 | 17.67 | 6 | C |
| ATOM | 556 | N | GLN | A | 76 | −0.990 | 27.187 | 32.318 | 1.00 | 21.66 | 7 | N |
| ATOM | 557 | CA | GLN | A | 76 | −0.778 | 26.987 | 33.756 | 1.00 | 21.72 | 6 | C |
| ATOM | 558 | C | GLN | A | 76 | −2.124 | 26.931 | 34.468 | 1.00 | 22.32 | 6 | C |
| ATOM | 559 | O | GLN | A | 76 | −2.367 | 25.991 | 35.241 | 1.00 | 23.61 | 8 | O |
| ATOM | 560 | CB | GLN | A | 76 | 0.156 | 28.057 | 34.340 | 1.00 | 20.67 | 6 | C |
| ATOM | 561 | CG | GLN | A | 76 | 0.700 | 27.683 | 35.729 | 1.00 | 20.28 | 6 | C |
| ATOM | 562 | CD | GLN | A | 76 | 1.544 | 26.424 | 35.663 | 1.00 | 20.05 | 6 | C |
| ATOM | 563 | OE1 | GLN | A | 76 | 2.454 | 26.331 | 34.841 | 1.00 | 20.80 | 8 | O |
| ATOM | 564 | NE2 | GLN | A | 76 | 1.283 | 25.413 | 36.476 | 1.00 | 20.51 | 7 | N |
| ATOM | 565 | N | TYR | A | 77 | −3.025 | 27.885 | 34.222 | 1.00 | 21.54 | 7 | N |
| ATOM | 566 | CA | TYR | A | 77 | −4.362 | 27.854 | 34.805 | 1.00 | 22.83 | 6 | C |
| ATOM | 567 | C | TYR | A | 77 | −4.974 | 26.478 | 34.559 | 1.00 | 23.93 | 6 | C |
| ATOM | 568 | O | TYR | A | 77 | −5.345 | 25.767 | 35.497 | 1.00 | 25.67 | 8 | O |
| ATOM | 569 | CB | TYR | A | 77 | −5.281 | 28.973 | 34.256 | 1.00 | 20.59 | 6 | C |
| ATOM | 570 | CG | TYR | A | 77 | −6.737 | 28.725 | 34.595 | 1.00 | 20.16 | 6 | C |
| ATOM | 571 | CD1 | TYR | A | 77 | −7.307 | 28.990 | 35.838 | 1.00 | 19.43 | 6 | C |
| ATOM | 572 | CD2 | TYR | A | 77 | −7.531 | 28.124 | 33.623 | 1.00 | 20.20 | 6 | C |
| ATOM | 573 | CE1 | TYR | A | 77 | −8.625 | 28.645 | 36.101 | 1.00 | 20.19 | 6 | C |
| ATOM | 574 | CE2 | TYR | A | 77 | −8.846 | 27.796 | 33.880 | 1.00 | 19.65 | 6 | C |
| ATOM | 575 | CZ | TYR | A | 77 | −9.391 | 28.056 | 35.123 | 1.00 | 19.11 | 6 | C |
| ATOM | 576 | OH | TYR | A | 77 | −10.698 | 27.720 | 35.313 | 1.00 | 19.26 | 8 | O |
| ATOM | 577 | N | ILE | A | 78 | −4.993 | 26.011 | 33.303 | 1.00 | 23.48 | 7 | N |
| ATOM | 578 | CA | ILE | A | 78 | −5.553 | 24.696 | 33.031 | 1.00 | 24.75 | 6 | C |
| ATOM | 579 | C | ILE | A | 78 | −4.871 | 23.622 | 33.881 | 1.00 | 24.56 | 6 | C |
| ATOM | 580 | O | ILE | A | 78 | −5.576 | 22.739 | 34.385 | 1.00 | 22.23 | 8 | O |
| ATOM | 581 | CB | ILE | A | 78 | −5.508 | 24.290 | 31.546 | 1.00 | 24.41 | 6 | C |
| ATOM | 582 | CG1 | ILE | A | 78 | −6.109 | 25.404 | 30.697 | 1.00 | 24.65 | 6 | C |
| ATOM | 583 | CG2 | ILE | A | 78 | −6.248 | 22.973 | 31.349 | 1.00 | 24.29 | 6 | C |
| ATOM | 584 | CD1 | ILE | A | 78 | −6.084 | 25.172 | 29.207 | 1.00 | 24.15 | 6 | C |
| ATOM | 585 | N | ILE | A | 79 | −3.542 | 23.675 | 33.975 | 1.00 | 25.05 | 7 | N |
| ATOM | 586 | CA | ILE | A | 79 | −2.824 | 22.700 | 34.781 | 1.00 | 25.66 | 6 | C |
| ATOM | 587 | C | ILE | A | 79 | −3.261 | 22.812 | 36.242 | 1.00 | 26.91 | 6 | C |
| ATOM | 588 | O | ILE | A | 79 | −3.813 | 21.869 | 36.803 | 1.00 | 27.41 | 8 | O |
| ATOM | 589 | CB | ILE | A | 79 | −1.293 | 22.830 | 34.664 | 1.00 | 25.49 | 6 | C |
| ATOM | 590 | CG1 | ILE | A | 79 | −0.767 | 22.600 | 33.247 | 1.00 | 24.63 | 6 | C |
| ATOM | 591 | CG2 | ILE | A | 79 | −0.661 | 21.817 | 35.628 | 1.00 | 24.28 | 6 | C |
| ATOM | 592 | CD1 | ILE | A | 79 | 0.636 | 23.153 | 33.038 | 1.00 | 24.70 | 6 | C |
| ATOM | 593 | N | ASP | A | 80 | −3.211 | 24.000 | 36.834 | 1.00 | 28.62 | 7 | N |
| ATOM | 594 | CA | ASP | A | 80 | −3.578 | 24.223 | 38.220 | 1.00 | 29.91 | 6 | C |
| ATOM | 595 | C | ASP | A | 80 | −5.009 | 23.831 | 38.569 | 1.00 | 30.38 | 6 | C |
| ATOM | 596 | O | ASP | A | 80 | −5.287 | 23.292 | 39.647 | 1.00 | 30.30 | 8 | O |
| ATOM | 597 | CB | ASP | A | 80 | −3.474 | 25.714 | 38.591 | 1.00 | 30.68 | 6 | C |
| ATOM | 598 | CG | ASP | A | 80 | −2.087 | 26.277 | 38.430 | 1.00 | 31.07 | 6 | C |
| ATOM | 599 | OD1 | ASP | A | 80 | −1.122 | 25.489 | 38.310 | 1.00 | 30.77 | 8 | O |
| ATOM | 600 | OD2 | ASP | A | 80 | −1.951 | 27.518 | 38.415 | 1.00 | 31.39 | 8 | O |
| ATOM | 601 | N | ASN | A | 81 | −5.922 | 24.166 | 37.657 | 1.00 | 28.48 | 7 | N |
| ATOM | 602 | CA | ASN | A | 81 | −7.342 | 23.956 | 37.797 | 1.00 | 27.10 | 6 | C |
| ATOM | 603 | C | ASN | A | 81 | −7.738 | 22.510 | 37.576 | 1.00 | 27.44 | 6 | C |
| ATOM | 604 | O | ASN | A | 81 | −8.858 | 22.095 | 37.860 | 1.00 | 25.88 | 8 | O |
| ATOM | 605 | CB | ASN | A | 81 | −8.064 | 24.764 | 36.692 | 1.00 | 26.97 | 6 | C |
| ATOM | 606 | CG | ASN | A | 81 | −9.556 | 24.852 | 36.910 | 1.00 | 26.16 | 6 | C |
| ATOM | 607 | OD1 | ASN | A | 81 | −9.936 | 25.213 | 38.018 | 1.00 | 26.37 | 8 | O |
| ATOM | 608 | ND2 | ASN | A | 81 | −10.385 | 24.575 | 35.920 | 1.00 | 25.24 | 7 | N |
| ATOM | 609 | N | LYS | A | 82 | −6.816 | 21.740 | 37.028 | 1.00 | 27.92 | 7 | N |
| ATOM | 610 | CA | LYS | A | 82 | −6.943 | 20.370 | 36.609 | 1.00 | 28.87 | 6 | C |
| ATOM | 611 | C | LYS | A | 82 | −8.171 | 20.162 | 35.722 | 1.00 | 28.70 | 6 | C |
| ATOM | 612 | O | LYS | A | 82 | −8.800 | 19.107 | 35.708 | 1.00 | 28.98 | 8 | O |
| ATOM | 613 | CB | LYS | A | 82 | −6.773 | 19.289 | 37.648 | 1.00 | 30.46 | 6 | C |
| ATOM | 614 | CG | LYS | A | 82 | −7.215 | 19.418 | 39.072 | 1.00 | 31.11 | 6 | C |
| ATOM | 615 | CD | LYS | A | 82 | −6.175 | 18.839 | 40.038 | 1.00 | 31.43 | 6 | C |
| ATOM | 616 | CE | LYS | A | 82 | −6.323 | 18.231 | 41.298 | 1.00 | 42.55 | 6 | C |
| ATOM | 617 | NZ | LYS | A | 82 | −6.739 | 19.021 | 42.455 | 1.00 | 42.93 | 7 | N |
| ATOM | 618 | N | GLY | A | 83 | −8.348 | 21.082 | 34.763 | 1.00 | 28.83 | 7 | N |
| ATOM | 619 | CA | GLY | A | 83 | −9.338 | 20.926 | 33.731 | 1.00 | 26.57 | 6 | C |
| ATOM | 620 | C | GLY | A | 83 | −9.818 | 22.202 | 33.069 | 1.00 | 26.03 | 6 | C |
| ATOM | 621 | O | GLY | A | 83 | −9.640 | 23.329 | 33.515 | 1.00 | 25.30 | 8 | O |
| ATOM | 622 | N | ILE | A | 84 | −10.463 | 21.962 | 31.920 | 1.00 | 25.05 | 7 | N |
| ATOM | 623 | CA | ILE | A | 84 | −11.126 | 23.031 | 31.179 | 1.00 | 22.45 | 6 | C |
| ATOM | 624 | C | ILE | A | 84 | −12.381 | 22.432 | 30.531 | 1.00 | 22.16 | 6 | C |
| ATOM | 625 | O | ILE | A | 84 | −12.339 | 21.353 | 29.972 | 1.00 | 22.15 | 8 | O |
| ATOM | 626 | CB | ILE | A | 84 | −10.183 | 23.680 | 30.163 | 1.00 | 22.23 | 6 | C |
| ATOM | 627 | CG1 | ILE | A | 84 | −10.934 | 24.726 | 29.339 | 1.00 | 21.20 | 6 | C |
| ATOM | 628 | CG2 | ILE | A | 84 | −9.548 | 22.630 | 29.259 | 1.00 | 19.22 | 6 | C |
| ATOM | 629 | CD1 | ILE | A | 84 | −10.037 | 25.790 | 28.744 | 1.00 | 22.01 | 6 | C |
| ATOM | 630 | N | ASP | A | 85 | −13.461 | 23.206 | 30.653 | 1.00 | 21.30 | 7 | N |
| ATOM | 631 | CA | ASP | A | 85 | −14.748 | 22.812 | 30.128 | 1.00 | 22.61 | 6 | C |
| ATOM | 632 | C | ASP | A | 85 | −14.765 | 22.874 | 28.602 | 1.00 | 23.42 | 6 | C |
| ATOM | 633 | O | ASP | A | 85 | −14.005 | 23.685 | 28.050 | 1.00 | 22.85 | 8 | O |
| ATOM | 634 | CB | ASP | A | 85 | −15.893 | 23.715 | 30.609 | 1.00 | 22.02 | 6 | C |
| ATOM | 635 | CG | ASP | A | 85 | −16.143 | 23.584 | 32.102 | 1.00 | 21.23 | 6 | C |
| ATOM | 636 | OD1 | ASP | A | 85 | −15.879 | 22.489 | 32.632 | 1.00 | 21.06 | 8 | O |
| ATOM | 637 | OD2 | ASP | A | 85 | −16.524 | 24.587 | 32.716 | 1.00 | 20.77 | 8 | O |
| ATOM | 638 | N | SER | A | 86 | −15.791 | 22.266 | 28.028 | 1.00 | 22.72 | 7 | N |
| ATOM | 639 | CA | SER | A | 86 | −15.974 | 22.390 | 26.589 | 1.00 | 22.22 | 6 | C |
| ATOM | 640 | C | SER | A | 86 | −16.579 | 23.765 | 26.329 | 1.00 | 22.57 | 6 | C |
| ATOM | 641 | O | SER | A | 86 | −17.156 | 24.386 | 27.230 | 1.00 | 21.45 | 8 | O |
| ATOM | 642 | CB | SER | A | 86 | −16.962 | 21.329 | 26.089 | 1.00 | 22.24 | 6 | C |
| ATOM | 643 | OG | SER | A | 86 | −18.221 | 21.694 | 26.665 | 1.00 | 24.14 | 8 | O |
| ATOM | 644 | N | ASP | A | 87 | −16.496 | 24.155 | 25.060 | 1.00 | 21.28 | 7 | N |
| ATOM | 645 | CA | ASP | A | 87 | −17.094 | 25.413 | 24.621 | 1.00 | 22.89 | 6 | C |
| ATOM | 646 | C | ASP | A | 87 | −18.615 | 25.302 | 24.720 | 1.00 | 23.24 | 6 | C |
| ATOM | 647 | O | ASP | A | 87 | −19.305 | 26.271 | 25.045 | 1.00 | 22.12 | 8 | O |
| ATOM | 648 | CB | ASP | A | 87 | −16.627 | 25.675 | 23.193 | 1.00 | 22.42 | 6 | C |
| ATOM | 649 | CG | ASP | A | 87 | −17.321 | 26.845 | 22.531 | 1.00 | 23.15 | 6 | C |
| ATOM | 650 | OD1 | ASP | A | 87 | −18.233 | 26.628 | 21.708 | 1.00 | 22.34 | 8 | O |
| ATOM | 651 | OD2 | ASP | A | 87 | −16.921 | 27.979 | 22.869 | 1.00 | 23.10 | 8 | O |
| ATOM | 652 | N | ALA | A | 88 | −19.143 | 24.102 | 24.484 | 1.00 | 24.49 | 7 | N |
| ATOM | 653 | CA | ALA | A | 88 | −20.577 | 23.865 | 24.611 | 1.00 | 26.40 | 6 | C |
| ATOM | 654 | C | ALA | A | 88 | −20.989 | 24.094 | 26.071 | 1.00 | 26.92 | 6 | C |
| ATOM | 655 | O | ALA | A | 88 | −21.998 | 24.796 | 26.177 | 1.00 | 27.55 | 8 | O |
| ATOM | 656 | CB | ALA | A | 88 | −20.972 | 22.499 | 24.100 | 1.00 | 25.55 | 6 | C |
| ATOM | 657 | N | SER | A | 89 | −20.309 | 23.597 | 27.077 | 1.00 | 27.31 | 7 | N |
| ATOM | 658 | CA | SER | A | 89 | −20.611 | 23.775 | 28.479 | 1.00 | 29.30 | 6 | C |
| ATOM | 659 | C | SER | A | 89 | −20.529 | 25.235 | 28.967 | 1.00 | 29.01 | 6 | C |
| ATOM | 660 | O | SER | A | 89 | −21.163 | 25.665 | 29.935 | 1.00 | 30.36 | 8 | O |
| ATOM | 661 | CB | SER | A | 89 | −19.626 | 23.022 | 29.377 | 1.00 | 29.91 | 6 | C |
| ATOM | 662 | OG | SER | A | 89 | −18.430 | 22.558 | 28.819 | 1.00 | 33.23 | 8 | O |
| ATOM | 663 | N | TYR | A | 90 | −19.551 | 25.966 | 28.461 | 1.00 | 28.62 | 7 | N |
| ATOM | 664 | CA | TYR | A | 90 | −19.202 | 27.320 | 28.921 | 1.00 | 26.48 | 6 | C |
| ATOM | 665 | C | TYR | A | 90 | −18.961 | 28.148 | 27.669 | 1.00 | 25.65 | 6 | C |
| ATOM | 666 | O | TYR | A | 90 | −17.836 | 28.215 | 27.159 | 1.00 | 23.88 | 8 | O |
| ATOM | 667 | CB | TYR | A | 90 | −17.941 | 27.091 | 29.736 | 1.00 | 26.03 | 6 | C |
| ATOM | 668 | CG | TYR | A | 90 | −17.539 | 28.168 | 30.704 | 1.00 | 26.25 | 6 | C |
| ATOM | 669 | CD1 | TYR | A | 90 | −17.977 | 29.477 | 30.578 | 1.00 | 26.03 | 6 | C |
| ATOM | 670 | CD2 | TYR | A | 90 | −16.666 | 27.868 | 31.743 | 1.00 | 25.94 | 6 | C |
| ATOM | 671 | CE1 | TYR | A | 90 | −17.583 | 30.446 | 31.483 | 1.00 | 25.96 | 6 | C |
| ATOM | 672 | CE2 | TYR | A | 90 | −16.273 | 28.826 | 32.651 | 1.00 | 25.75 | 6 | C |
| ATOM | 673 | CZ | TYR | A | 90 | −16.735 | 30.117 | 32.513 | 1.00 | 24.66 | 6 | C |
| ATOM | 674 | OH | TYR | A | 90 | −16.314 | 31.063 | 33.406 | 1.00 | 24.26 | 8 | O |
| ATOM | 675 | N | PRO | A | 91 | −20.042 | 28.682 | 27.119 | 1.00 | 24.16 | 7 | N |
| ATOM | 676 | CA | PRO | A | 91 | −20.007 | 29.366 | 25.844 | 1.00 | 22.38 | 6 | C |
| ATOM | 677 | C | PRO | A | 91 | −19.347 | 30.720 | 25.876 | 1.00 | 21.65 | 6 | C |
| ATOM | 678 | O | PRO | A | 91 | −19.439 | 31.456 | 26.846 | 1.00 | 20.61 | 8 | O |
| ATOM | 679 | CB | PRO | A | 91 | −21.480 | 29.418 | 25.434 | 1.00 | 22.64 | 6 | C |
| ATOM | 680 | CG | PRO | A | 91 | −22.181 | 29.567 | 26.755 | 1.00 | 24.11 | 6 | C |
| ATOM | 681 | CD | PRO | A | 91 | −21.423 | 28.630 | 27.675 | 1.00 | 24.27 | 6 | C |
| ATOM | 682 | N | TYR | A | 92 | −18.793 | 31.090 | 24.731 | 1.00 | 20.39 | 7 | N |
| ATOM | 683 | CA | TYR | A | 92 | −18.093 | 32.335 | 24.525 | 1.00 | 20.26 | 6 | C |
| ATOM | 684 | C | TYR | A | 92 | −19.023 | 33.528 | 24.394 | 1.00 | 20.72 | 6 | C |
| ATOM | 685 | O | TYR | A | 92 | −19.872 | 33.569 | 23.519 | 1.00 | 20.05 | 8 | O |
| ATOM | 686 | CB | TYR | A | 92 | −17.252 | 32.237 | 23.241 | 1.00 | 21.16 | 6 | C |
| ATOM | 687 | CG | TYR | A | 92 | −16.470 | 33.527 | 23.106 | 1.00 | 22.39 | 6 | C |
| ATOM | 688 | CD1 | TYR | A | 92 | −15.462 | 33.789 | 24.043 | 1.00 | 21.79 | 6 | C |
| ATOM | 689 | CD2 | TYR | A | 92 | −16.702 | 34.443 | 22.100 | 1.00 | 22.14 | 6 | C |
| ATOM | 690 | CE1 | TYR | A | 92 | −14.712 | 34.939 | 23.964 | 1.00 | 22.27 | 6 | C |
| ATOM | 691 | CE2 | TYR | A | 92 | −15.927 | 35.588 | 22.013 | 1.00 | 21.89 | 6 | C |
| ATOM | 692 | CZ | TYR | A | 92 | −14.917 | 35.820 | 22.927 | 1.00 | 22.27 | 6 | C |
| ATOM | 693 | OH | TYR | A | 92 | −14.191 | 36.982 | 22.847 | 1.00 | 21.59 | 8 | O |
| ATOM | 694 | N | LYS | A | 93 | −18.868 | 34.487 | 25.301 | 1.00 | 22.15 | 7 | N |
| ATOM | 695 | CA | LYS | A | 93 | −19.746 | 35.642 | 25.399 | 1.00 | 22.54 | 6 | C |
| ATOM | 696 | C | LYS | A | 93 | −19.141 | 36.957 | 24.958 | 1.00 | 20.79 | 6 | C |
| ATOM | 697 | O | LYS | A | 93 | −19.855 | 37.966 | 24.905 | 1.00 | 18.83 | 8 | O |
| ATOM | 698 | CB | LYS | A | 93 | −20.182 | 35.851 | 26.879 | 1.00 | 22.50 | 6 | C |
| ATOM | 699 | CG | LYS | A | 93 | −20.731 | 34.551 | 27.457 | 1.00 | 23.95 | 6 | C |
| ATOM | 700 | CD | LYS | A | 93 | −22.024 | 34.153 | 26.785 | 1.00 | 25.94 | 6 | C |
| ATOM | 701 | CE | LYS | A | 93 | −23.143 | 35.160 | 27.030 | 1.00 | 26.02 | 6 | C |
| ATOM | 702 | NZ | LYS | A | 93 | −24.449 | 34.566 | 26.604 | 1.00 | 26.53 | 7 | N |
| ATOM | 703 | N | ALA | A | 94 | −17.851 | 36.909 | 24.649 | 1.00 | 19.89 | 7 | N |
| ATOM | 704 | CA | ALA | A | 94 | −17.122 | 38.112 | 24.253 | 1.00 | 19.34 | 6 | C |
| ATOM | 705 | C | ALA | A | 94 | −17.224 | 39.241 | 25.282 | 1.00 | 19.71 | 6 | C |
| ATOM | 706 | O | ALA | A | 94 | −17.261 | 40.424 | 24.927 | 1.00 | 17.68 | 8 | O |
| ATOM | 707 | CB | ALA | A | 94 | −17.595 | 38.584 | 22.886 | 1.00 | 18.14 | 6 | C |
| ATOM | 708 | N | MET | A | 95 | −16.953 | 38.903 | 26.534 | 1.00 | 21.05 | 7 | N |
| ATOM | 709 | CA | MET | A | 95 | −16.907 | 39.847 | 27.637 | 1.00 | 24.45 | 6 | C |
| ATOM | 710 | C | MET | A | 95 | −16.495 | 39.171 | 28.940 | 1.00 | 24.65 | 6 | C |
| ATOM | 711 | O | MET | A | 95 | −16.800 | 37.972 | 29.115 | 1.00 | 24.37 | 8 | O |
| ATOM | 712 | CB | MET | A | 95 | −18.298 | 40.477 | 27.838 | 1.00 | 26.81 | 6 | C |
| ATOM | 713 | CG | MET | A | 95 | −19.359 | 39.424 | 28.209 | 1.00 | 26.79 | 6 | C |
| ATOM | 714 | SD | MET | A | 95 | −21.029 | 40.005 | 27.871 | 1.00 | 30.72 | 16 | S |
| ATOM | 715 | CE | MET | A | 95 | −21.972 | 39.122 | 29.099 | 1.00 | 29.49 | 6 | C |
| ATOM | 716 | N | ASP | A | 96 | −15.962 | 39.940 | 29.876 | 1.00 | 25.99 | 7 | N |
| ATOM | 717 | CA | ASP | A | 96 | −15.590 | 39.382 | 31.169 | 1.00 | 27.97 | 6 | C |
| ATOM | 718 | C | ASP | A | 96 | −16.818 | 38.900 | 31.926 | 1.00 | 28.50 | 6 | C |
| ATOM | 719 | O | ASP | A | 96 | −17.838 | 39.575 | 31.939 | 1.00 | 28.69 | 8 | O |
| ATOM | 720 | CB | ASP | A | 96 | −14.807 | 40.443 | 31.955 | 1.00 | 29.78 | 6 | C |
| ATOM | 721 | CG | ASP | A | 96 | −13.626 | 40.858 | 31.075 | 1.00 | 32.20 | 6 | C |
| ATOM | 722 | OD1 | ASP | A | 96 | −12.870 | 39.954 | 30.664 | 1.00 | 31.39 | 8 | O |
| ATOM | 723 | OD2 | ASP | A | 96 | −13.479 | 42.037 | 30.718 | 1.00 | 34.54 | 8 | O |
| ATOM | 724 | N | LEU | A | 97 | −16.733 | 37.756 | 32.569 | 1.00 | 28.84 | 7 | N |
| ATOM | 725 | CA | LEU | A | 97 | −17.800 | 37.158 | 33.359 | 1.00 | 29.61 | 6 | C |
| ATOM | 726 | C | LEU | A | 97 | −17.083 | 36.598 | 34.609 | 1.00 | 29.21 | 6 | C |
| ATOM | 727 | O | LEU | A | 97 | −15.877 | 36.345 | 34.565 | 1.00 | 27.54 | 8 | O |
| ATOM | 728 | CB | LEU | A | 97 | −18.575 | 36.028 | 32.728 | 1.00 | 29.72 | 6 | C |
| ATOM | 729 | CG | LEU | A | 97 | −19.250 | 35.651 | 31.447 | 1.00 | 30.88 | 6 | C |
| ATOM | 730 | CD1 | LEU | A | 97 | −18.989 | 34.166 | 31.073 | 1.00 | 30.72 | 6 | C |
| ATOM | 731 | CD2 | LEU | A | 97 | −20.784 | 35.654 | 31.423 | 1.00 | 31.20 | 6 | C |
| ATOM | 732 | N | LYS | A | 98 | −17.831 | 36.399 | 35.685 | 1.00 | 27.84 | 7 | N |
| ATOM | 733 | CA | LYS | A | 98 | −17.252 | 35.788 | 36.880 | 1.00 | 27.09 | 6 | C |
| ATOM | 734 | C | LYS | A | 98 | −16.829 | 34.380 | 36.493 | 1.00 | 27.55 | 6 | C |
| ATOM | 735 | O | LYS | A | 98 | −17.465 | 33.788 | 35.609 | 1.00 | 27.68 | 8 | O |
| ATOM | 736 | CB | LYS | A | 98 | −18.341 | 35.689 | 37.956 | 1.00 | 28.62 | 6 | C |
| ATOM | 737 | CG | LYS | A | 98 | −17.548 | 34.862 | 39.319 | 1.00 | 39.81 | 6 | C |
| ATOM | 738 | CD | LYS | A | 98 | −18.150 | 35.112 | 40.684 | 1.00 | 41.59 | 6 | C |
| ATOM | 739 | CE | LYS | A | 98 | −17.708 | 34.025 | 41.672 | 1.00 | 42.03 | 6 | C |
| ATOM | 740 | NZ | LYS | A | 98 | −18.353 | 34.248 | 43.004 | 1.00 | 43.18 | 7 | N |
| ATOM | 741 | N | CYS | A | 99 | −15.809 | 33.815 | 37.114 | 1.00 | 27.58 | 7 | N |
| ATOM | 742 | CA | CYS | A | 99 | −15.426 | 32.454 | 36.750 | 1.00 | 27.48 | 6 | C |
| ATOM | 743 | C | CYS | A | 99 | −16.656 | 31.566 | 36.957 | 1.00 | 29.46 | 6 | C |
| ATOM | 744 | O | CYS | A | 99 | −17.302 | 31.659 | 38.006 | 1.00 | 29.32 | 8 | O |
| ATOM | 745 | CB | CYS | A | 99 | −14.270 | 31.949 | 37.606 | 1.00 | 26.02 | 6 | C |
| ATOM | 746 | SG | CYS | A | 99 | −13.792 | 30.277 | 37.192 | 1.00 | 25.37 | 16 | S |
| ATOM | 747 | N | GLN | A | 100 | −16.959 | 30.725 | 35.978 | 1.00 | 29.42 | 7 | N |
| ATOM | 748 | CA | GLN | A | 100 | −18.055 | 29.776 | 36.114 | 1.00 | 29.28 | 6 | C |
| ATOM | 749 | C | GLN | A | 100 | −17.610 | 28.341 | 35.855 | 1.00 | 29.19 | 6 | C |
| ATOM | 750 | O | GLN | A | 100 | −18.429 | 27.530 | 35.421 | 1.00 | 27.17 | 8 | O |
| ATOM | 751 | CB | GLN | A | 100 | −19.174 | 30.147 | 35.136 | 1.00 | 29.06 | 6 | C |
| ATOM | 752 | CG | GLN | A | 100 | −20.484 | 29.931 | 35.520 | 1.00 | 46.34 | 6 | C |
| ATOM | 753 | CD | GLN | A | 100 | −21.458 | 29.877 | 34.342 | 1.00 | 48.63 | 6 | C |
| ATOM | 754 | OE1 | GLN | A | 100 | −22.029 | 28.797 | 34.129 | 1.00 | 50.35 | 8 | O |
| ATOM | 755 | NE2 | GLN | A | 100 | −21.562 | 30.990 | 33.624 | 1.00 | 48.58 | 7 | N |
| ATOM | 756 | N | TYR | A | 101 | −16.315 | 28.054 | 36.012 | 1.00 | 29.02 | 7 | N |
| ATOM | 757 | CA | TYR | A | 101 | −15.839 | 26.699 | 35.731 | 1.00 | 29.46 | 6 | C |
| ATOM | 758 | C | TYR | A | 101 | −16.705 | 25.689 | 36.471 | 1.00 | 30.77 | 6 | C |
| ATOM | 759 | O | TYR | A | 101 | −17.258 | 26.008 | 37.519 | 1.00 | 31.93 | 8 | O |
| ATOM | 760 | CB | TYR | A | 101 | −14.358 | 26.530 | 36.072 | 1.00 | 28.65 | 6 | C |
| ATOM | 761 | CG | TYR | A | 101 | −13.859 | 25.101 | 35.991 | 1.00 | 29.07 | 6 | C |
| ATOM | 762 | CD1 | TYR | A | 101 | −13.748 | 24.489 | 34.746 | 1.00 | 28.89 | 6 | C |
| ATOM | 763 | CD2 | TYR | A | 101 | −13.525 | 24.361 | 37.121 | 1.00 | 28.14 | 6 | C |
| ATOM | 764 | CE1 | TYR | A | 101 | −13.326 | 23.179 | 34.646 | 1.00 | 30.02 | 6 | C |
| ATOM | 765 | CE2 | TYR | A | 101 | −13.073 | 23.069 | 37.018 | 1.00 | 28.90 | 6 | C |
| ATOM | 766 | CZ | TYR | A | 101 | −12.981 | 22.472 | 35.780 | 1.00 | 29.41 | 6 | C |
| ATOM | 767 | OH | TYR | A | 101 | −12.548 | 21.172 | 35.646 | 1.00 | 29.60 | 8 | O |
| ATOM | 768 | N | ASP | A | 102 | −16.887 | 24.507 | 35.897 | 1.00 | 31.15 | 7 | N |
| ATOM | 769 | CA | ASP | A | 102 | −17.678 | 23.447 | 36.506 | 1.00 | 30.68 | 6 | C |
| ATOM | 770 | C | ASP | A | 102 | −17.059 | 22.126 | 36.092 | 1.00 | 30.56 | 6 | C |
| ATOM | 771 | O | ASP | A | 102 | −17.072 | 21.747 | 34.917 | 1.00 | 30.33 | 8 | O |
| ATOM | 772 | CB | ASP | A | 102 | −19.150 | 23.555 | 36.110 | 1.00 | 30.88 | 6 | C |
| ATOM | 773 | CG | ASP | A | 102 | −20.018 | 22.423 | 36.634 | 1.00 | 30.35 | 6 | C |
| ATOM | 774 | OD1 | ASP | A | 102 | −19.510 | 21.461 | 37.230 | 1.00 | 29.42 | 8 | O |
| ATOM | 775 | OD2 | ASP | A | 102 | −21.244 | 22.454 | 36.436 | 1.00 | 31.11 | 8 | O |
| ATOM | 776 | N | SER | A | 103 | −16.549 | 21.356 | 37.039 | 1.00 | 31.50 | 7 | N |
| ATOM | 777 | CA | SER | A | 103 | −15.935 | 20.064 | 36.756 | 1.00 | 32.32 | 6 | C |
| ATOM | 778 | C | SER | A | 103 | −16.854 | 19.076 | 36.062 | 1.00 | 33.09 | 6 | C |
| ATOM | 779 | O | SER | A | 103 | −16.332 | 18.213 | 35.345 | 1.00 | 31.59 | 8 | O |
| ATOM | 780 | CB | SER | A | 103 | −15.320 | 19.428 | 38.008 | 1.00 | 32.37 | 6 | C |
| ATOM | 781 | OG | SER | A | 103 | −16.173 | 19.527 | 39.138 | 1.00 | 31.98 | 8 | O |
| ATOM | 782 | N | LYS | A | 104 | −18.179 | 19.151 | 36.160 | 1.00 | 35.76 | 7 | N |
| ATOM | 783 | CA | LYS | A | 104 | −19.053 | 18.202 | 35.463 | 1.00 | 37.80 | 6 | C |
| ATOM | 784 | C | LYS | A | 104 | −18.851 | 18.236 | 33.950 | 1.00 | 37.33 | 6 | C |
| ATOM | 785 | O | LYS | A | 104 | −19.038 | 17.241 | 33.251 | 1.00 | 35.66 | 8 | O |
| ATOM | 786 | CB | LYS | A | 104 | −20.524 | 18.471 | 35.798 | 1.00 | 39.75 | 6 | C |
| ATOM | 787 | CG | LYS | A | 104 | −21.532 | 17.557 | 35.121 | 1.00 | 42.27 | 6 | C |
| ATOM | 788 | CD | LYS | A | 104 | −22.980 | 17.947 | 35.402 | 1.00 | 43.54 | 6 | C |
| ATOM | 789 | CE | LYS | A | 104 | −23.957 | 16.905 | 34.865 | 1.00 | 43.83 | 6 | C |
| ATOM | 790 | NZ | LYS | A | 104 | −23.982 | 15.669 | 35.708 | 1.00 | 45.07 | 7 | N |
| ATOM | 791 | N | TYR | A | 105 | −18.536 | 19.414 | 33.409 | 1.00 | 37.05 | 7 | N |
| ATOM | 792 | CA | TYR | A | 105 | −18.382 | 19.612 | 31.979 | 1.00 | 36.81 | 6 | C |
| ATOM | 793 | C | TYR | A | 105 | −16.943 | 19.729 | 31.504 | 1.00 | 35.99 | 6 | C |
| ATOM | 794 | O | TYR | A | 105 | −16.673 | 20.287 | 30.429 | 1.00 | 33.12 | 8 | O |
| ATOM | 795 | CB | TYR | A | 105 | −19.227 | 20.836 | 31.611 | 1.00 | 38.96 | 6 | C |
| ATOM | 796 | CG | TYR | A | 105 | −20.694 | 20.672 | 31.965 | 1.00 | 41.55 | 6 | C |
| ATOM | 797 | CD1 | TYR | A | 105 | −21.210 | 21.176 | 33.149 | 1.00 | 42.60 | 6 | C |
| ATOM | 798 | CD2 | TYR | A | 105 | −21.552 | 19.983 | 31.125 | 1.00 | 42.60 | 6 | C |
| ATOM | 799 | CE1 | TYR | A | 105 | −22.541 | 21.005 | 33.483 | 1.00 | 44.25 | 6 | C |
| ATOM | 800 | CE2 | TYR | A | 105 | −22.886 | 19.811 | 31.445 | 1.00 | 43.94 | 6 | C |
| ATOM | 801 | CZ | TYR | A | 105 | −23.379 | 20.323 | 32.627 | 1.00 | 44.66 | 6 | C |
| ATOM | 802 | OH | TYR | A | 105 | −24.711 | 20.121 | 32.925 | 1.00 | 45.49 | 8 | O |
| ATOM | 803 | N | ARG | A | 106 | −15.982 | 19.220 | 32.284 | 1.00 | 35.58 | 7 | N |
| ATOM | 804 | CA | ARG | A | 106 | −14.585 | 19.274 | 31.881 | 1.00 | 35.22 | 6 | C |
| ATOM | 805 | C | ARG | A | 106 | −14.428 | 18.483 | 30.575 | 1.00 | 33.66 | 6 | C |
| ATOM | 806 | O | ARG | A | 106 | −14.848 | 17.317 | 30.544 | 1.00 | 32.71 | 8 | O |
| ATOM | 807 | CB | ARG | A | 106 | −13.596 | 18.692 | 32.885 | 1.00 | 37.05 | 6 | C |
| ATOM | 808 | CG | ARG | A | 106 | −12.195 | 18.554 | 32.329 | 1.00 | 39.12 | 6 | C |
| ATOM | 809 | CD | ARG | A | 106 | −11.197 | 17.832 | 33.187 | 1.00 | 41.38 | 6 | C |
| ATOM | 810 | NE | ARG | A | 106 | −11.744 | 17.036 | 34.270 | 1.00 | 43.12 | 7 | N |
| ATOM | 811 | CZ | ARG | A | 106 | −11.774 | 17.509 | 35.515 | 1.00 | 45.18 | 6 | C |
| ATOM | 812 | NH1 | ARG | A | 106 | −11.276 | 18.723 | 35.741 | 1.00 | 46.74 | 7 | N |
| ATOM | 813 | NH2 | ARG | A | 106 | −12.286 | 16.820 | 36.516 | 1.00 | 45.39 | 7 | N |
| ATOM | 814 | N | ALA | A | 107 | −13.727 | 19.086 | 29.616 | 1.00 | 30.82 | 7 | N |
| ATOM | 815 | CA | ALA | A | 107 | −13.555 | 18.345 | 28.358 | 1.00 | 29.53 | 6 | C |
| ATOM | 816 | C | ALA | A | 107 | −12.092 | 18.012 | 28.133 | 1.00 | 28.50 | 6 | C |
| ATOM | 817 | O | ALA | A | 107 | −11.796 | 17.176 | 27.292 | 1.00 | 29.94 | 8 | O |
| ATOM | 818 | CB | ALA | A | 107 | −14.125 | 19.200 | 27.231 | 1.00 | 29.16 | 6 | C |
| ATOM | 819 | N | ALA | A | 108 | −11.144 | 18.630 | 28.833 | 1.00 | 28.04 | 7 | N |
| ATOM | 820 | CA | ALA | A | 108 | −9.729 | 18.348 | 28.637 | 1.00 | 27.60 | 6 | C |
| ATOM | 821 | C | ALA | A | 108 | −8.870 | 18.770 | 29.829 | 1.00 | 27.82 | 6 | C |
| ATOM | 822 | O | ALA | A | 108 | −9.330 | 19.529 | 30.674 | 1.00 | 28.33 | 8 | O |
| ATOM | 823 | CB | ALA | A | 108 | −9.165 | 19.093 | 27.420 | 1.00 | 26.00 | 6 | C |
| ATOM | 824 | N | THR | A | 109 | −7.643 | 18.270 | 29.843 | 1.00 | 28.37 | 7 | N |
| ATOM | 825 | CA | THR | A | 109 | −6.685 | 18.648 | 30.886 | 1.00 | 30.12 | 6 | C |
| ATOM | 826 | C | THR | A | 109 | −5.297 | 18.948 | 30.328 | 1.00 | 29.00 | 6 | C |
| ATOM | 827 | O | THR | A | 109 | −5.029 | 18.586 | 29.179 | 1.00 | 30.28 | 8 | O |
| ATOM | 828 | CB | THR | A | 109 | −6.537 | 17.485 | 31.890 | 1.00 | 30.39 | 6 | C |
| ATOM | 829 | OG1 | THR | A | 109 | −5.493 | 17.830 | 32.806 | 1.00 | 33.72 | 8 | O |
| ATOM | 830 | CG2 | THR | A | 109 | −6.181 | 16.207 | 31.158 | 1.00 | 29.62 | 6 | C |
| ATOM | 831 | N | CYS | A | 110 | −4.387 | 19.478 | 31.134 | 1.00 | 28.87 | 7 | N |
| ATOM | 832 | CA | CYS | A | 110 | −3.019 | 19.727 | 30.697 | 1.00 | 28.09 | 6 | C |
| ATOM | 833 | C | CYS | A | 110 | −2.025 | 19.414 | 31.803 | 1.00 | 29.30 | 6 | C |
| ATOM | 834 | O | CYS | A | 110 | −2.238 | 19.782 | 32.961 | 1.00 | 29.59 | 8 | O |
| ATOM | 835 | CB | CYS | A | 110 | −2.861 | 21.183 | 30.233 | 1.00 | 27.11 | 6 | C |
| ATOM | 836 | SG | CYS | A | 110 | −1.262 | 21.583 | 29.499 | 1.00 | 24.00 | 16 | S |
| ATOM | 837 | N | SER | A | 111 | −0.928 | 18.739 | 31.481 | 1.00 | 30.28 | 7 | N |
| ATOM | 838 | CA | SER | A | 111 | 0.080 | 18.404 | 32.470 | 1.00 | 30.99 | 6 | C |
| ATOM | 839 | C | SER | A | 111 | 1.276 | 19.348 | 32.408 | 1.00 | 31.45 | 6 | C |
| ATOM | 840 | O | SER | A | 111 | 1.940 | 19.484 | 33.425 | 1.00 | 31.58 | 8 | O |
| ATOM | 841 | CB | SER | A | 111 | 0.647 | 16.999 | 32.216 | 1.00 | 31.38 | 6 | C |
| ATOM | 842 | OG | SER | A | 111 | −0.405 | 16.192 | 31.722 | 1.00 | 33.93 | 8 | O |
| ATOM | 843 | N | LYS | A | 112 | 1.610 | 19.806 | 31.195 | 1.00 | 31.38 | 7 | N |
| ATOM | 844 | CA | LYS | A | 112 | 2.797 | 20.625 | 31.012 | 1.00 | 30.62 | 6 | C |
| ATOM | 845 | C | LYS | A | 112 | 2.704 | 21.538 | 29.795 | 1.00 | 29.73 | 6 | C |
| ATOM | 846 | O | LYS | A | 112 | 2.062 | 21.186 | 28.803 | 1.00 | 30.73 | 8 | O |
| ATOM | 847 | CB | LYS | A | 112 | 4.050 | 19.753 | 30.870 | 1.00 | 31.67 | 6 | C |
| ATOM | 848 | CG | LYS | A | 112 | 4.243 | 18.944 | 29.614 | 1.00 | 33.08 | 6 | C |
| ATOM | 849 | CD | LYS | A | 112 | 5.683 | 18.790 | 29.174 | 1.00 | 33.67 | 6 | C |
| ATOM | 850 | CE | LYS | A | 112 | 6.584 | 18.089 | 30.163 | 1.00 | 34.48 | 6 | C |
| ATOM | 851 | NZ | LYS | A | 112 | 8.159 | 18.200 | 29.948 | 1.00 | 46.23 | 7 | N |
| ATOM | 852 | N | TYR | A | 113 | 3.314 | 22.706 | 29.842 | 1.00 | 28.03 | 7 | N |
| ATOM | 853 | CA | TYR | A | 113 | 3.366 | 23.489 | 28.593 | 1.00 | 26.64 | 6 | C |
| ATOM | 854 | C | TYR | A | 113 | 4.837 | 23.658 | 28.265 | 1.00 | 26.44 | 6 | C |
| ATOM | 855 | O | TYR | A | 113 | 5.682 | 23.518 | 29.154 | 1.00 | 24.84 | 8 | O |
| ATOM | 856 | CB | TYR | A | 113 | 2.541 | 24.744 | 28.680 | 1.00 | 25.60 | 6 | C |
| ATOM | 857 | CG | TYR | A | 113 | 3.057 | 25.824 | 29.582 | 1.00 | 25.86 | 6 | C |
| ATOM | 858 | CD1 | TYR | A | 113 | 3.926 | 26.743 | 29.006 | 1.00 | 26.36 | 6 | C |
| ATOM | 859 | CD2 | TYR | A | 113 | 2.712 | 25.972 | 30.909 | 1.00 | 25.51 | 6 | C |
| ATOM | 860 | CE1 | TYR | A | 113 | 4.454 | 27.795 | 29.739 | 1.00 | 25.64 | 6 | C |
| ATOM | 861 | CE2 | TYR | A | 113 | 3.210 | 27.029 | 31.641 | 1.00 | 26.27 | 6 | C |
| ATOM | 862 | CZ | TYR | A | 113 | 4.076 | 27.924 | 31.052 | 1.00 | 26.55 | 6 | C |
| ATOM | 863 | OH | TYR | A | 113 | 4.579 | 28.979 | 31.776 | 1.00 | 29.49 | 8 | O |
| ATOM | 864 | N | THR | A | 114 | 5.157 | 23.928 | 27.006 | 1.00 | 26.59 | 7 | N |
| ATOM | 865 | CA | THR | A | 114 | 6.540 | 24.120 | 26.585 | 1.00 | 27.79 | 6 | C |
| ATOM | 866 | C | THR | A | 114 | 6.647 | 25.343 | 25.662 | 1.00 | 27.79 | 6 | C |
| ATOM | 867 | O | THR | A | 114 | 5.999 | 25.432 | 24.622 | 1.00 | 26.59 | 8 | O |
| ATOM | 868 | CB | THR | A | 114 | 7.118 | 22.925 | 25.800 | 1.00 | 28.47 | 6 | C |
| ATOM | 869 | OG1 | THR | A | 114 | 7.022 | 21.716 | 26.565 | 1.00 | 28.45 | 8 | O |
| ATOM | 870 | CG2 | THR | A | 114 | 8.548 | 23.203 | 25.363 | 1.00 | 27.01 | 6 | C |
| ATOM | 871 | N | GLU | A | 115 | 7.546 | 26.244 | 26.027 | 1.00 | 28.03 | 7 | N |
| ATOM | 872 | CA | GLU | A | 115 | 7.806 | 27.444 | 25.253 | 1.00 | 28.31 | 6 | C |
| ATOM | 873 | C | GLU | A | 115 | 9.077 | 27.287 | 24.415 | 1.00 | 26.45 | 6 | C |
| ATOM | 874 | O | GLU | A | 115 | 10.065 | 26.828 | 24.966 | 1.00 | 24.11 | 8 | O |
| ATOM | 875 | CB | GLU | A | 115 | 8.101 | 28.637 | 26.157 | 1.00 | 30.49 | 6 | C |
| ATOM | 876 | CG | GLU | A | 115 | 7.372 | 28.589 | 27.485 | 1.00 | 34.67 | 6 | C |
| ATOM | 877 | CD | GLU | A | 115 | 7.705 | 29.814 | 28.317 | 1.00 | 36.29 | 6 | C |
| ATOM | 878 | OE1 | GLU | A | 115 | 6.957 | 30.802 | 28.181 | 1.00 | 38.25 | 8 | O |
| ATOM | 879 | OE2 | GLU | A | 115 | 8.696 | 29.769 | 29.069 | 1.00 | 39.15 | 8 | O |
| ATOM | 880 | N | LEU | A | 116 | 9.018 | 27.700 | 23.167 | 1.00 | 25.14 | 7 | N |
| ATOM | 881 | CA | LEU | A | 116 | 10.135 | 27.608 | 22.263 | 1.00 | 25.16 | 6 | C |
| ATOM | 882 | C | LEU | A | 116 | 11.054 | 28.824 | 22.320 | 1.00 | 24.89 | 6 | C |
| ATOM | 883 | O | LEU | A | 116 | 10.641 | 29.898 | 22.746 | 1.00 | 23.04 | 8 | O |
| ATOM | 884 | CB | LEU | A | 116 | 9.659 | 27.416 | 20.826 | 1.00 | 25.43 | 6 | C |
| ATOM | 885 | CG | LEU | A | 116 | 8.784 | 26.187 | 20.579 | 1.00 | 25.71 | 6 | C |
| ATOM | 886 | CD1 | LEU | A | 116 | 8.481 | 26.106 | 19.100 | 1.00 | 25.82 | 6 | C |
| ATOM | 887 | CD2 | LEU | A | 116 | 9.421 | 24.914 | 21.103 | 1.00 | 27.18 | 6 | C |
| ATOM | 888 | N | PRO | A | 117 | 12.304 | 28.641 | 21.888 | 1.00 | 24.28 | 7 | N |
| ATOM | 889 | CA | PRO | A | 117 | 13.293 | 29.697 | 21.877 | 1.00 | 23.98 | 6 | C |
| ATOM | 890 | C | PRO | A | 117 | 12.868 | 30.877 | 21.019 | 1.00 | 22.49 | 6 | C |
| ATOM | 891 | O | PRO | A | 117 | 12.365 | 30.759 | 19.915 | 1.00 | 22.50 | 8 | O |
| ATOM | 892 | CB | PRO | A | 117 | 14.596 | 29.038 | 21.412 | 1.00 | 24.47 | 6 | C |
| ATOM | 893 | CG | PRO | A | 117 | 14.319 | 27.579 | 21.360 | 1.00 | 25.14 | 6 | C |
| ATOM | 894 | CD | PRO | A | 117 | 12.829 | 27.364 | 21.356 | 1.00 | 24.60 | 6 | C |
| ATOM | 895 | N | TYR | A | 118 | 13.002 | 32.087 | 21.547 | 1.00 | 21.36 | 7 | N |
| ATOM | 896 | CA | TYR | A | 118 | 12.691 | 33.318 | 20.861 | 1.00 | 21.59 | 6 | C |
| ATOM | 897 | C | TYR | A | 118 | 13.203 | 33.390 | 19.431 | 1.00 | 21.06 | 6 | C |
| ATOM | 898 | O | TYR | A | 118 | 14.396 | 33.232 | 19.195 | 1.00 | 19.33 | 8 | O |
| ATOM | 899 | CB | TYR | A | 118 | 13.326 | 34.528 | 21.591 | 1.00 | 22.46 | 6 | C |
| ATOM | 900 | CG | TYR | A | 118 | 12.895 | 35.829 | 20.938 | 1.00 | 23.39 | 6 | C |
| ATOM | 901 | CD1 | TYR | A | 118 | 13.564 | 36.453 | 19.901 | 1.00 | 22.31 | 6 | C |
| ATOM | 902 | CD2 | TYR | A | 118 | 11.711 | 36.406 | 21.407 | 1.00 | 23.67 | 6 | C |
| ATOM | 903 | CE1 | TYR | A | 118 | 13.086 | 37.624 | 19.337 | 1.00 | 22.81 | 6 | C |
| ATOM | 904 | CE2 | TYR | A | 118 | 11.229 | 37.578 | 20.862 | 1.00 | 21.86 | 6 | C |
| ATOM | 905 | CZ | TYR | A | 118 | 11.907 | 38.179 | 19.831 | 1.00 | 22.33 | 6 | C |
| ATOM | 906 | OH | TYR | A | 118 | 11.373 | 39.337 | 19.327 | 1.00 | 20.93 | 8 | O |
| ATOM | 907 | N | GLY | A | 119 | 12.328 | 33.728 | 18.484 | 1.00 | 20.73 | 7 | N |
| ATOM | 908 | CA | GLY | A | 119 | 12.645 | 33.926 | 17.100 | 1.00 | 19.44 | 6 | C |
| ATOM | 909 | C | GLY | A | 119 | 13.143 | 32.750 | 16.293 | 1.00 | 19.69 | 6 | C |
| ATOM | 910 | O | GLY | A | 119 | 13.361 | 32.930 | 15.084 | 1.00 | 19.60 | 8 | O |
| ATOM | 911 | N | ARG | A | 120 | 13.223 | 31.549 | 16.857 | 1.00 | 19.84 | 7 | N |
| ATOM | 912 | CA | ARG | A | 120 | 13.778 | 30.391 | 16.166 | 1.00 | 19.67 | 6 | C |
| ATOM | 913 | C | ARG | A | 120 | 12.741 | 29.672 | 15.321 | 1.00 | 19.06 | 6 | C |
| ATOM | 914 | O | ARG | A | 120 | 11.965 | 28.800 | 15.691 | 1.00 | 16.52 | 8 | O |
| ATOM | 915 | CB | ARG | A | 120 | 14.532 | 29.510 | 17.162 | 1.00 | 20.06 | 6 | C |
| ATOM | 916 | CG | ARG | A | 120 | 15.877 | 30.088 | 17.624 | 1.00 | 19.22 | 6 | C |
| ATOM | 917 | CD | ARG | A | 120 | 16.895 | 30.186 | 16.507 | 1.00 | 18.90 | 6 | C |
| ATOM | 918 | NE | ARG | A | 120 | 17.321 | 28.905 | 15.948 | 1.00 | 19.77 | 7 | N |
| ATOM | 919 | CZ | ARG | A | 120 | 18.239 | 28.106 | 16.500 | 1.00 | 20.36 | 6 | C |
| ATOM | 920 | NH1 | ARG | A | 120 | 18.578 | 26.951 | 15.950 | 1.00 | 21.29 | 7 | N |
| ATOM | 921 | NH2 | ARG | A | 120 | 18.834 | 28.449 | 17.634 | 1.00 | 19.03 | 7 | N |
| ATOM | 922 | N | GLU | A | 121 | 12.814 | 30.009 | 14.021 | 1.00 | 19.45 | 7 | N |
| ATOM | 923 | CA | GLU | A | 121 | 11.799 | 29.549 | 13.063 | 1.00 | 19.61 | 6 | C |
| ATOM | 924 | C | GLU | A | 121 | 11.992 | 28.082 | 12.709 | 1.00 | 20.60 | 6 | C |
| ATOM | 925 | O | GLU | A | 121 | 11.037 | 27.345 | 12.414 | 1.00 | 20.89 | 8 | O |
| ATOM | 926 | CB | GLU | A | 121 | 11.747 | 30.492 | 11.865 | 1.00 | 18.24 | 6 | C |
| ATOM | 927 | CG | GLU | A | 121 | 11.051 | 31.811 | 12.220 | 1.00 | 18.86 | 6 | C |
| ATOM | 928 | CD | GLU | A | 121 | 10.797 | 32.663 | 10.989 | 1.00 | 19.96 | 6 | C |
| ATOM | 929 | OE1 | GLU | A | 121 | 9.770 | 32.439 | 10.315 | 1.00 | 19.81 | 8 | O |
| ATOM | 930 | OE2 | GLU | A | 121 | 11.600 | 33.557 | 10.629 | 1.00 | 20.40 | 8 | O |
| ATOM | 931 | N | ASP | A | 122 | 13.241 | 27.626 | 12.716 | 1.00 | 19.89 | 7 | N |
| ATOM | 932 | CA | ASP | A | 122 | 13.586 | 26.218 | 12.529 | 1.00 | 19.72 | 6 | C |
| ATOM | 933 | C | ASP | A | 122 | 13.072 | 25.400 | 13.705 | 1.00 | 19.29 | 6 | C |
| ATOM | 934 | O | ASP | A | 122 | 12.484 | 24.338 | 13.521 | 1.00 | 19.52 | 8 | O |
| ATOM | 935 | CB | ASP | A | 122 | 15.108 | 26.049 | 12.434 | 1.00 | 20.29 | 6 | C |
| ATOM | 936 | CG | ASP | A | 122 | 15.867 | 26.754 | −13.552 | 1.00 | 20.53 | 6 | C |
| ATOM | 937 | OD1 | ASP | A | 122 | 15.432 | 27.748 | 14.169 | 1.00 | 19.33 | 8 | O |
| ATOM | 938 | OD2 | ASP | A | 122 | 16.985 | 26.284 | 13.843 | 1.00 | 20.00 | 8 | O |
| ATOM | 939 | N | VAL | A | 123 | 13.218 | 25.896 | 14.932 | 1.00 | 19.63 | 7 | N |
| ATOM | 940 | CA | VAL | A | 123 | 12.683 | 25.167 | 16.082 | 1.00 | 20.15 | 6 | C |
| ATOM | 941 | C | VAL | A | 123 | 11.160 | 25.117 | 16.069 | 1.00 | 19.82 | 6 | C |
| ATOM | 942 | O | VAL | A | 123 | 10.581 | 24.080 | 16.393 | 1.00 | 21.01 | 8 | O |
| ATOM | 943 | CB | VAL | A | 123 | 13.224 | 25.708 | 17.409 | 1.00 | 20.72 | 6 | C |
| ATOM | 944 | CG1 | VAL | A | 123 | 12.741 | 24.865 | 18.586 | 1.00 | 19.61 | 6 | C |
| ATOM | 945 | CG2 | VAL | A | 123 | 14.751 | 25.666 | 17.374 | 1.00 | 21.00 | 6 | C |
| ATOM | 946 | N | LEU | A | 124 | 10.482 | 26.192 | 15.684 | 1.00 | 19.45 | 7 | N |
| ATOM | 947 | CA | LEU | A | 124 | 9.034 | 26.204 | 15.543 | 1.00 | 17.29 | 6 | C |
| ATOM | 948 | C | LEU | A | 124 | 8.587 | 25.205 | 14.466 | 1.00 | 16.81 | 6 | C |
| ATOM | 949 | O | LEU | A | 124 | 7.617 | 24.483 | 14.630 | 1.00 | 14.06 | 8 | O |
| ATOM | 950 | CB | LEU | A | 124 | 8.528 | 27.595 | 15.109 | 1.00 | 16.10 | 6 | C |
| ATOM | 951 | CG | LEU | A | 124 | 6.994 | 27.693 | 14.930 | 1.00 | 15.09 | 6 | C |
| ATOM | 952 | CD1 | LEU | A | 124 | 6.303 | 27.248 | 16.214 | 1.00 | 13.15 | 6 | C |
| ATOM | 953 | CD2 | LEU | A | 124 | 6.568 | 29.112 | 14.549 | 1.00 | 14.30 | 6 | C |
| ATOM | 954 | N | LYS | A | 125 | 9.288 | 25.174 | 13.336 | 1.00 | 17.30 | 7 | N |
| ATOM | 955 | CA | LYS | A | 125 | 9.003 | 24.247 | 12.254 | 1.00 | 19.43 | 6 | C |
| ATOM | 956 | C | LYS | A | 125 | 9.005 | 22.811 | 12.770 | 1.00 | 20.30 | 6 | C |
| ATOM | 957 | O | LYS | A | 125 | 8.017 | 22.090 | 12.617 | 1.00 | 18.99 | 8 | O |
| ATOM | 958 | CB | LYS | A | 125 | 9.998 | 24.386 | 11.103 | 1.00 | 20.04 | 6 | C |
| ATOM | 959 | CG | LYS | A | 125 | 9.724 | 23.440 | 9.948 | 1.00 | 21.22 | 6 | C |
| ATOM | 960 | CD | LYS | A | 125 | 10.641 | 23.668 | 8.758 | 1.00 | 22.50 | 6 | C |
| ATOM | 961 | CE | LYS | A | 125 | 10.871 | 22.373 | 7.989 | 1.00 | 23.62 | 6 | C |
| ATOM | 962 | NZ | LYS | A | 125 | 11.506 | 22.596 | 6.661 | 1.00 | 23.20 | 7 | N |
| ATOM | 963 | N | GLU | A | 126 | 10.085 | 22.462 | 13.474 | 1.00 | 21.92 | 7 | N |
| ATOM | 964 | CA | GLU | A | 126 | 10.163 | 21.145 | 14.093 | 1.00 | 25.66 | 6 | C |
| ATOM | 965 | C | GLU | A | 126 | 9.047 | 20.904 | 15.103 | 1.00 | 24.45 | 6 | C |
| ATOM | 966 | O | GLU | A | 126 | 8.436 | 19.816 | 15.077 | 1.00 | 23.61 | 8 | O |
| ATOM | 967 | CB | GLU | A | 126 | 11.552 | 20.915 | 14.703 | 1.00 | 29.31 | 6 | C |
| ATOM | 968 | CG | GLU | A | 126 | 11.616 | 19.797 | 15.737 | 1.00 | 33.75 | 6 | C |
| ATOM | 969 | CD | GLU | A | 126 | 13.042 | 19.478 | 16.159 | 1.00 | 37.53 | 6 | C |
| ATOM | 970 | OE1 | GLU | A | 126 | 13.252 | 18.340 | 16.645 | 1.00 | 39.17 | 8 | O |
| ATOM | 971 | OE2 | GLU | A | 126 | 13.944 | 20.342 | 16.014 | 1.00 | 38.33 | 8 | O |
| ATOM | 972 | N | ALA | A | 127 | 8.716 | 21.855 | 15.976 | 1.00 | 22.89 | 7 | N |
| ATOM | 973 | CA | ALA | A | 127 | 7.595 | 21.610 | 16.892 | 1.00 | 22.42 | 6 | C |
| ATOM | 974 | C | ALA | A | 127 | 6.263 | 21.373 | 16.189 | 1.00 | 21.97 | 6 | C |
| ATOM | 975 | O | ALA | A | 127 | 5.478 | 20.498 | 16.574 | 1.00 | 22.41 | 8 | O |
| ATOM | 976 | CB | ALA | A | 127 | 7.437 | 22.761 | 17.870 | 1.00 | 22.20 | 6 | C |
| ATOM | 977 | N | VAL | A | 128 | 5.945 | 22.173 | 15.183 | 1.00 | 21.64 | 7 | N |
| ATOM | 978 | CA | VAL | A | 128 | 4.736 | 22.073 | 14.402 | 1.00 | 21.56 | 6 | C |
| ATOM | 979 | C | VAL | A | 128 | 4.639 | 20.693 | 13.766 | 1.00 | 22.59 | 6 | C |
| ATOM | 980 | O | VAL | A | 128 | 3.553 | 20.109 | 13.756 | 1.00 | 23.86 | 8 | O |
| ATOM | 981 | CB | VAL | A | 128 | 4.638 | 23.147 | 13.294 | 1.00 | 21.77 | 6 | C |
| ATOM | 982 | CG1 | VAL | A | 128 | 3.486 | 22.862 | 12.337 | 1.00 | 19.24 | 6 | C |
| ATOM | 983 | CG2 | VAL | A | 128 | 4.452 | 24.530 | 13.925 | 1.00 | 20.70 | 6 | C |
| ATOM | 984 | N | ALA | A | 129 | 5.729 | 20.203 | 13.181 | 1.00 | 22.80 | 7 | N |
| ATOM | 985 | CA | ALA | A | 129 | 5.674 | 18.880 | 12.565 | 1.00 | 22.75 | 6 | C |
| ATOM | 986 | C | ALA | A | 129 | 5.511 | 17.787 | 13.616 | 1.00 | 22.71 | 6 | C |
| ATOM | 987 | O | ALA | A | 129 | 4.780 | 16.822 | 13.389 | 1.00 | 23.44 | 8 | O |
| ATOM | 988 | CB | ALA | A | 129 | 6.910 | 18.527 | 11.740 | 1.00 | 20.00 | 6 | C |
| ATOM | 989 | N | ASN | A | 130 | 6.328 | 17.837 | 14.658 | 1.00 | 24.07 | 7 | N |
| ATOM | 990 | CA | ASN | A | 130 | 6.382 | 16.693 | 15.561 | 1.00 | 25.89 | 6 | C |
| ATOM | 991 | C | ASN | A | 130 | 5.479 | 16.733 | 16.768 | 1.00 | 25.42 | 6 | C |
| ATOM | 992 | O | ASN | A | 130 | 5.295 | 15.626 | 17.269 | 1.00 | 27.13 | 8 | O |
| ATOM | 993 | CB | ASN | A | 130 | 7.836 | 16.485 | 16.015 | 1.00 | 27.47 | 6 | C |
| ATOM | 994 | CG | ASN | A | 130 | 8.720 | 16.119 | 14.840 | 1.00 | 29.35 | 6 | C |
| ATOM | 995 | OD1 | ASN | A | 130 | 8.249 | 15.475 | 13.893 | 1.00 | 30.70 | 8 | O |
| ATOM | 996 | ND2 | ASN | A | 130 | 9.956 | 16.584 | 14.865 | 1.00 | 29.01 | 7 | N |
| ATOM | 997 | N | LYS | A | 131 | 5.028 | 17.871 | 17.268 | 1.00 | 24.28 | 7 | N |
| ATOM | 998 | CA | LYS | A | 131 | 4.168 | 17.931 | 18.435 | 1.00 | 23.51 | 6 | C |
| ATOM | 999 | C | LYS | A | 131 | 2.697 | 18.234 | 18.126 | 1.00 | 22.64 | 6 | C |
| ATOM | 1000 | O | LYS | A | 131 | 1.799 | 17.601 | 18.697 | 1.00 | 21.17 | 8 | O |
| ATOM | 1001 | CB | LYS | A | 131 | 4.641 | 19.061 | 19.367 | 1.00 | 24.90 | 6 | C |
| ATOM | 1002 | CG | LYS | A | 131 | 5.606 | 18.688 | 20.465 | 1.00 | 26.15 | 6 | C |
| ATOM | 1003 | CD | LYS | A | 131 | 6.781 | 17.881 | 20.010 | 1.00 | 27.86 | 6 | C |
| ATOM | 1004 | CE | LYS | A | 131 | 7.879 | 17.742 | 21.050 | 1.00 | 29.68 | 6 | C |
| ATOM | 1005 | NZ | LYS | A | 131 | 7.867 | 16.402 | 21.691 | 1.00 | 29.96 | 7 | N |
| ATOM | 1006 | N | GLY | A | 132 | 2.484 | 19.237 | 17.279 | 1.00 | 20.55 | 7 | N |
| ATOM | 1007 | CA | GLY | A | 132 | 1.147 | 19.657 | 16.863 | 1.00 | 20.25 | 6 | C |
| ATOM | 1008 | C | GLY | A | 132 | 1.136 | 21.181 | 16.695 | 1.00 | 19.24 | 6 | C |
| ATOM | 1009 | O | GLY | A | 132 | 2.181 | 21.836 | 16.816 | 1.00 | 19.27 | 8 | O |
| ATOM | 1010 | N | PRO | A | 133 | −0.036 | 21.745 | 16.470 | 1.00 | 17.37 | 7 | N |
| ATOM | 1011 | CA | PRO | A | 133 | −0.218 | 23.175 | 16.371 | 1.00 | 17.05 | 6 | C |
| ATOM | 1012 | C | PRO | A | 133 | 0.364 | 23.942 | 17.552 | 1.00 | 16.38 | 6 | C |
| ATOM | 1013 | O | PRO | A | 133 | 0.274 | 23.546 | 18.715 | 1.00 | 13.98 | 8 | O |
| ATOM | 1014 | CB | PRO | A | 133 | −1.737 | 23.341 | 16.283 | 1.00 | 16.46 | 6 | C |
| ATOM | 1015 | CG | PRO | A | 133 | −2.213 | 22.045 | 15.676 | 1.00 | 16.62 | 6 | C |
| ATOM | 1016 | CD | PRO | A | 133 | −1.323 | 21.016 | 16.320 | 1.00 | 17.85 | 6 | C |
| ATOM | 1017 | N | VAL | A | 134 | 0.996 | 25.086 | 17.280 | 1.00 | 15.75 | 7 | N |
| ATOM | 1018 | CA | VAL | A | 134 | 1.680 | 25.863 | 18.309 | 1.00 | 15.90 | 6 | C |
| ATOM | 1019 | C | VAL | A | 134 | 1.117 | 27.259 | 18.499 | 1.00 | 15.95 | 6 | C |
| ATOM | 1020 | O | VAL | A | 134 | 0.833 | 27.984 | 17.540 | 1.00 | 16.80 | 8 | O |
| ATOM | 1021 | CB | VAL | A | 134 | 3.190 | 25.953 | 18.021 | 1.00 | 14.77 | 6 | C |
| ATOM | 1022 | CG1 | VAL | A | 134 | 3.933 | 26.824 | 19.031 | 1.00 | 12.60 | 6 | C |
| ATOM | 1023 | CG2 | VAL | A | 134 | 3.821 | 24.565 | 17.985 | 1.00 | 15.02 | 6 | C |
| ATOM | 1024 | N | SER | A | 135 | 0.904 | 27.605 | 19.762 | 1.00 | 16.37 | 7 | N |
| ATOM | 1025 | CA | SER | A | 135 | 0.384 | 28.926 | 20.111 | 1.00 | 15.61 | 6 | C |
| ATOM | 1026 | C | SER | A | 135 | 1.488 | 29.963 | 19.988 | 1.00 | 15.26 | 6 | C |
| ATOM | 1027 | O | SER | A | 135 | 2.618 | 29.763 | 20.487 | 1.00 | 14.54 | 8 | O |
| ATOM | 1028 | CB | SER | A | 135 | −0.163 | 28.944 | 21.539 | 1.00 | 15.74 | 6 | C |
| ATOM | 1029 | OG | SER | A | 135 | −1.155 | 27.954 | 21.737 | 1.00 | 14.29 | 8 | O |
| ATOM | 1030 | N | VAL | A | 136 | 1.199 | 31.002 | 19.191 | 1.00 | 13.15 | 7 | N |
| ATOM | 1031 | CA | VAL | A | 136 | 2.175 | 32.073 | 19.017 | 1.00 | 12.51 | 6 | C |
| ATOM | 1032 | C | VAL | A | 136 | 1.472 | 33.420 | 19.084 | 1.00 | 12.80 | 6 | C |
| ATOM | 1033 | O | VAL | A | 136 | 0.239 | 33.475 | 18.907 | 1.00 | 12.74 | 8 | O |
| ATOM | 1034 | CB | VAL | A | 136 | 2.863 | 32.017 | 17.633 | 1.00 | 11.40 | 6 | C |
| ATOM | 1035 | CG1 | VAL | A | 136 | 3.675 | 30.719 | 17.490 | 1.00 | 11.79 | 6 | C |
| ATOM | 1036 | CG2 | VAL | A | 136 | 1.830 | 32.124 | 16.528 | 1.00 | 10.93 | 6 | C |
| ATOM | 1037 | N | GLY | A | 137 | 2.269 | 34.469 | 19.213 | 1.00 | 13.13 | 7 | N |
| ATOM | 1038 | CA | GLY | A | 137 | 1.721 | 35.816 | 19.066 | 1.00 | 12.05 | 6 | C |
| ATOM | 1039 | C | GLY | A | 137 | 2.240 | 36.390 | 17.740 | 1.00 | 14.27 | 6 | C |
| ATOM | 1040 | O | GLY | A | 137 | 3.323 | 36.044 | 17.240 | 1.00 | 13.27 | 8 | O |
| ATOM | 1041 | N | VAL | A | 138 | 1.515 | 37.378 | 17.188 | 1.00 | 13.76 | 7 | N |
| ATOM | 1042 | CA | VAL | A | 138 | 1.974 | 38.086 | 16.014 | 1.00 | 15.53 | 6 | C |
| ATOM | 1043 | C | VAL | A | 138 | 1.616 | 39.566 | 16.237 | 1.00 | 16.13 | 6 | C |
| ATOM | 1044 | O | VAL | A | 138 | 0.827 | 39.920 | 17.116 | 1.00 | 15.77 | 8 | O |
| ATOM | 1045 | CB | VAL | A | 138 | 1.451 | 37.665 | 14.629 | 1.00 | 15.17 | 6 | C |
| ATOM | 1046 | CG1 | VAL | A | 138 | 1.782 | 36.237 | 14.225 | 1.00 | 12.68 | 6 | C |
| ATOM | 1047 | CG2 | VAL | A | 138 | −0.070 | 37.808 | 14.646 | 1.00 | 15.85 | 6 | C |
| ATOM | 1048 | N | ASP | A | 139 | 2.312 | 40.405 | 15.510 | 1.00 | 16.45 | 7 | N |
| ATOM | 1049 | CA | ASP | A | 139 | 2.074 | 41.839 | 15.404 | 1.00 | 16.56 | 6 | C |
| ATOM | 1050 | C | ASP | A | 139 | 1.011 | 41.945 | 14.299 | 1.00 | 16.67 | 6 | C |
| ATOM | 1051 | O | ASP | A | 139 | 1.388 | 41.827 | 13.127 | 1.00 | 15.17 | 8 | O |
| ATOM | 1052 | CB | ASP | A | 139 | 3.295 | 42.642 | 14.970 | 1.00 | 14.71 | 6 | C |
| ATOM | 1053 | CG | ASP | A | 139 | 3.060 | 44.125 | 14.782 | 1.00 | 17.17 | 6 | C |
| ATOM | 1054 | OD1 | ASP | A | 139 | 1.962 | 44.621 | 15.138 | 1.00 | 17.02 | 8 | O |
| ATOM | 1055 | OD2 | ASP | A | 139 | 3.975 | 44.838 | 14.289 | 1.00 | 14.60 | 8 | O |
| ATOM | 1056 | N | ALA | A | 140 | −0.238 | 42.128 | 14.704 | 1.00 | 17.09 | 7 | N |
| ATOM | 1057 | CA | ALA | A | 140 | −1.331 | 42.201 | 13.745 | 1.00 | 17.51 | 6 | C |
| ATOM | 1058 | C | ALA | A | 140 | −1.852 | 43.633 | 13.590 | 1.00 | 19.31 | 6 | C |
| ATOM | 1059 | O | ALA | A | 140 | −2.924 | 43.808 | 12.982 | 1.00 | 18.42 | 8 | O |
| ATOM | 1060 | CB | ALA | A | 140 | −2.485 | 41.373 | 14.276 | 1.00 | 16.94 | 6 | C |
| ATOM | 1061 | N | ARG | A | 141 | −1.138 | 44.596 | 14.168 | 1.00 | 17.62 | 7 | N |
| ATOM | 1062 | CA | ARG | A | 141 | −1.604 | 45.973 | 14.121 | 1.00 | 21.70 | 6 | C |
| ATOM | 1063 | C | ARG | A | 141 | −1.295 | 46.741 | 12.861 | 1.00 | 21.24 | 6 | C |
| ATOM | 1064 | O | ARG | A | 141 | −0.491 | 47.683 | 12.908 | 1.00 | 23.78 | 8 | O |
| ATOM | 1065 | CB | ARG | A | 141 | −1.022 | 46.763 | 15.324 | 1.00 | 22.98 | 6 | C |
| ATOM | 1066 | CG | ARG | A | 141 | −1.524 | 46.205 | 16.646 | 1.00 | 25.16 | 6 | C |
| ATOM | 1067 | CD | ARG | A | 141 | −1.173 | 47.074 | 17.838 | 1.00 | 25.54 | 6 | C |
| ATOM | 1068 | NE | ARG | A | 141 | −1.756 | 46.493 | 19.054 | 1.00 | 26.83 | 7 | N |
| ATOM | 1069 | CZ | ARG | A | 141 | −1.457 | 46.813 | 20.306 | 1.00 | 27.15 | 6 | C |
| ATOM | 1070 | NH1 | ARG | A | 141 | −0.595 | 47.780 | 20.573 | 1.00 | 27.93 | 7 | N |
| ATOM | 1071 | NH2 | ARG | A | 141 | −2.027 | 46.200 | 21.335 | 1.00 | 28.15 | 7 | N |
| ATOM | 1072 | N | HIS | A | 142 | −1.664 | 46.307 | 11.680 | 1.00 | 20.53 | 7 | N |
| ATOM | 1073 | CA | HIS | A | 142 | −1.408 | 46.985 | 10.406 | 1.00 | 20.76 | 6 | C |
| ATOM | 1074 | C | HIS | A | 142 | −2.644 | 46.813 | 9.537 | 1.00 | 20.78 | 6 | C |
| ATOM | 1075 | O | HIS | A | 142 | −3.082 | 45.648 | 9.467 | 1.00 | 21.08 | 8 | O |
| ATOM | 1076 | CB | HIS | A | 142 | −0.180 | 46.315 | 9.723 | 1.00 | 20.65 | 6 | C |
| ATOM | 1077 | CG | HIS | A | 142 | 1.075 | 46.606 | 10.506 | 1.00 | 21.46 | 6 | C |
| ATOM | 1078 | ND1 | HIS | A | 142 | 1.623 | 45.689 | 11.384 | 1.00 | 20.82 | 7 | N |
| ATOM | 1079 | CD2 | HIS | A | 142 | 1.759 | 47.757 | 10.676 | 1.00 | 20.06 | 6 | C |
| ATOM | 1080 | CE1 | HIS | A | 142 | 2.636 | 46.251 | 12.001 | 1.00 | 20.89 | 6 | C |
| ATOM | 1081 | NE2 | HIS | A | 142 | 2.727 | 47.509 | 11.599 | 1.00 | 20.80 | 7 | N |
| ATOM | 1082 | N | PRO | A | 143 | −3.119 | 47.802 | 8.804 | 1.00 | 20.03 | 7 | N |
| ATOM | 1083 | CA | PRO | A | 143 | −4.245 | 47.631 | 7.897 | 1.00 | 20.34 | 6 | C |
| ATOM | 1084 | C | PRO | A | 143 | −4.094 | 46.365 | 7.082 | 1.00 | 18.65 | 6 | C |
| ATOM | 1085 | O | PRO | A | 143 | −5.029 | 45.598 | 6.930 | 1.00 | 17.09 | 8 | O |
| ATOM | 1086 | CB | PRO | A | 143 | −4.256 | 48.895 | 7.039 | 1.00 | 20.31 | 6 | C |
| ATOM | 1087 | CG | PRO | A | 143 | −3.706 | 49.932 | 7.978 | 1.00 | 19.99 | 6 | C |
| ATOM | 1088 | CD | PRO | A | 143 | −2.646 | 49.201 | 8.793 | 1.00 | 19.76 | 6 | C |
| ATOM | 1089 | N | SER | A | 144 | −2.938 | 46.186 | 6.498 | 1.00 | 17.95 | 7 | N |
| ATOM | 1090 | CA | SER | A | 144 | −2.419 | 45.007 | 5.852 | 1.00 | 18.06 | 6 | C |
| ATOM | 1091 | C | SER | A | 144 | −3.012 | 43.711 | 6.433 | 1.00 | 18.09 | 6 | C |
| ATOM | 1092 | O | SER | A | 144 | −3.293 | 42.752 | 5.698 | 1.00 | 17.56 | 8 | O |
| ATOM | 1093 | CB | SER | A | 144 | −0.995 | 45.087 | 6.439 | 1.00 | 18.08 | 6 | C |
| ATOM | 1094 | OG | SER | A | 144 | 0.052 | 44.459 | 5.893 | 1.00 | 18.27 | 8 | O |
| ATOM | 1095 | N | PHE | A | 145 | −2.861 | 43.559 | 7.756 | 1.00 | 16.72 | 7 | N |
| ATOM | 1096 | CA | PHE | A | 145 | −3.259 | 42.308 | 8.406 | 1.00 | 17.71 | 6 | C |
| ATOM | 1097 | C | PHE | A | 145 | −4.773 | 42.168 | 8.311 | 1.00 | 18.02 | 6 | C |
| ATOM | 1098 | O | PHE | A | 145 | −5.194 | 41.202 | 7.649 | 1.00 | 17.64 | 8 | O |
| ATOM | 1099 | CB | PHE | A | 145 | −2.743 | 42.211 | 9.839 | 1.00 | 17.73 | 6 | C |
| ATOM | 1100 | CG | PHE | A | 145 | −2.918 | 40.852 | 10.436 | 1.00 | 17.54 | 6 | C |
| ATOM | 1101 | CD1 | PHE | A | 145 | −1.838 | 39.999 | 10.535 | 1.00 | 18.10 | 6 | C |
| ATOM | 1102 | CD2 | PHE | A | 145 | −4.159 | 40.436 | 10.881 | 1.00 | 18.89 | 6 | C |
| ATOM | 1103 | CE1 | PHE | A | 145 | −1.973 | 38.737 | 11.089 | 1.00 | 18.91 | 6 | C |
| ATOM | 1104 | CE2 | PHE | A | 145 | −4.303 | 39.169 | 11.428 | 1.00 | 20.49 | 6 | C |
| ATOM | 1105 | CZ | PHE | A | 145 | −3.210 | 38.326 | 11.536 | 1.00 | 19.41 | 6 | C |
| ATOM | 1106 | N | PHE | A | 146 | −5.595 | 43.019 | 8.878 | 1.00 | 19.20 | 7 | N |
| ATOM | 1107 | CA | PHE | A | 146 | −7.053 | 42.937 | 8.812 | 1.00 | 22.75 | 6 | C |
| ATOM | 1108 | C | PHE | A | 146 | −7.573 | 42.683 | 7.404 | 1.00 | 22.08 | 6 | C |
| ATOM | 1109 | O | PHE | A | 146 | −8.406 | 41.808 | 7.137 | 1.00 | 21.21 | 8 | O |
| ATOM | 1110 | CB | PHE | A | 146 | −7.759 | 44.177 | 9.373 | 1.00 | 28.40 | 6 | C |
| ATOM | 1111 | CG | PHE | A | 146 | −9.255 | 44.207 | 9.497 | 1.00 | 34.22 | 6 | C |
| ATOM | 1112 | CD1 | PHE | A | 146 | −9.899 | 43.812 | 10.664 | 1.00 | 35.82 | 6 | C |
| ATOM | 1113 | CD2 | PHE | A | 146 | −10.054 | 44.647 | 8.454 | 1.00 | 35.66 | 6 | C |
| ATOM | 1114 | CE1 | PHE | A | 146 | −11.285 | 43.862 | 10.770 | 1.00 | 37.01 | 6 | C |
| ATOM | 1115 | CE2 | PHE | A | 146 | −11.426 | 44.687 | 8.544 | 1.00 | 36.85 | 6 | C |
| ATOM | 1116 | CZ | PHE | A | 146 | −12.054 | 44.301 | 9.711 | 1.00 | 37.74 | 6 | C |
| ATOM | 1117 | N | LEU | A | 147 | −6.970 | 43.271 | 6.376 | 1.00 | 19.53 | 7 | N |
| ATOM | 1118 | CA | LEU | A | 147 | −7.392 | 43.181 | 5.011 | 1.00 | 18.93 | 6 | C |
| ATOM | 1119 | C | LEU | A | 147 | −6.889 | 42.004 | 4.186 | 1.00 | 17.46 | 6 | C |
| ATOM | 1120 | O | LEU | A | 147 | −7.326 | 41.908 | 3.034 | 1.00 | 15.50 | 8 | O |
| ATOM | 1121 | CB | LEU | A | 147 | −6.967 | 44.484 | 4.283 | 1.00 | 18.33 | 6 | C |
| ATOM | 1122 | CG | LEU | A | 147 | −7.787 | 45.739 | 4.564 | 1.00 | 21.73 | 6 | C |
| ATOM | 1123 | CD1 | LEU | A | 147 | −8.298 | 45.872 | 5.978 | 1.00 | 21.70 | 6 | C |
| ATOM | 1124 | CD2 | LEU | A | 147 | −6.918 | 46.976 | 4.244 | 1.00 | 21.76 | 6 | C |
| ATOM | 1125 | N | TYR | A | 148 | −5.946 | 41.203 | 4.662 | 1.00 | 16.56 | 7 | N |
| ATOM | 1126 | CA | TYR | A | 148 | −5.434 | 40.069 | 3.905 | 1.00 | 16.70 | 6 | C |
| ATOM | 1127 | C | TYR | A | 148 | −6.501 | 39.170 | 3.289 | 1.00 | 16.77 | 6 | C |
| ATOM | 1128 | O | TYR | A | 148 | −7.432 | 38.781 | 4.023 | 1.00 | 17.96 | 8 | O |
| ATOM | 1129 | CB | TYR | A | 148 | −4.554 | 39.282 | 4.890 | 1.00 | 16.18 | 6 | C |
| ATOM | 1130 | CG | TYR | A | 148 | −4.168 | 37.916 | 4.354 | 1.00 | 17.56 | 6 | C |
| ATOM | 1131 | CD1 | TYR | A | 148 | −4.924 | 36.802 | 4.688 | 1.00 | 17.15 | 6 | C |
| ATOM | 1132 | CD2 | TYR | A | 148 | −3.071 | 37.745 | 3.528 | 1.00 | 16.54 | 6 | C |
| ATOM | 1133 | CE1 | TYR | A | 148 | −4.585 | 35.556 | 4.201 | 1.00 | 16.90 | 6 | C |
| ATOM | 1134 | CE2 | TYR | A | 148 | −2.684 | 36.493 | 3.081 | 1.00 | 17.60 | 6 | C |
| ATOM | 1135 | CZ | TYR | A | 148 | −3.470 | 35.401 | 3.411 | 1.00 | 17.72 | 6 | C |
| ATOM | 1136 | OH | TYR | A | 148 | −3.144 | 34.141 | 2.938 | 1.00 | 16.70 | 8 | O |
| ATOM | 1137 | N | ARG | A | 149 | −6.449 | 38.793 | 2.023 | 1.00 | 17.44 | 7 | N |
| ATOM | 1138 | CA | ARG | A | 149 | −7.430 | 37.898 | 1.446 | 1.00 | 19.25 | 6 | C |
| ATOM | 1139 | C | ARG | A | 149 | −6.752 | 36.576 | 1.073 | 1.00 | 19.34 | 6 | C |
| ATOM | 1140 | O | ARG | A | 149 | −7.286 | 35.499 | 1.367 | 1.00 | 18.38 | 8 | O |
| ATOM | 1141 | CB | ARG | A | 149 | −8.172 | 38.427 | 0.222 | 1.00 | 20.29 | 6 | C |
| ATOM | 1142 | CG | ARG | A | 149 | −8.986 | 39.688 | 0.387 | 1.00 | 21.48 | 6 | C |
| ATOM | 1143 | CD | ARG | A | 149 | −9.868 | 39.706 | 1.624 | 1.00 | 22.27 | 6 | C |
| ATOM | 1144 | NE | ARG | A | 149 | −10.893 | 38.659 | 1.602 | 1.00 | 22.65 | 7 | N |
| ATOM | 1145 | CZ | ARG | A | 149 | −11.624 | 38.242 | 2.623 | 1.00 | 23.37 | 6 | C |
| ATOM | 1146 | NH1 | ARG | A | 149 | −11.489 | 38.706 | 3.856 | 1.00 | 24.26 | 7 | N |
| ATOM | 1147 | NH2 | ARG | A | 149 | −12.530 | 37.284 | 2.420 | 1.00 | 22.64 | 7 | N |
| ATOM | 1148 | N | SER | A | 150 | −5.582 | 36.670 | 0.442 | 1.00 | 19.76 | 7 | N |
| ATOM | 1149 | CA | SER | A | 150 | −4.944 | 35.441 | −0.036 | 1.00 | 21.28 | 6 | C |
| ATOM | 1150 | C | SER | A | 150 | −3.462 | 35.561 | −0.308 | 1.00 | 21.01 | 6 | C |
| ATOM | 1151 | O | SER | A | 150 | −2.970 | 36.681 | −0.379 | 1.00 | 20.26 | 8 | O |
| ATOM | 1152 | CB | SER | A | 150 | −5.675 | 35.161 | −1.384 | 1.00 | 21.60 | 6 | C |
| ATOM | 1153 | OG | SER | A | 150 | −5.423 | 36.337 | −2.166 | 1.00 | 24.11 | 8 | O |
| ATOM | 1154 | N | GLY | A | 151 | −2.730 | 34.445 | −0.407 | 1.00 | 21.86 | 7 | N |
| ATOM | 1155 | CA | GLY | A | 151 | −1.303 | 34.525 | −0.711 | 1.00 | 21.56 | 6 | C |
| ATOM | 1156 | C | GLY | A | 151 | −0.437 | 34.476 | 0.542 | 1.00 | 22.52 | 6 | C |
| ATOM | 1157 | O | GLY | A | 151 | −0.940 | 34.152 | 1.618 | 1.00 | 23.36 | 8 | O |
| ATOM | 1158 | N | VAL | A | 152 | 0.863 | 34.739 | 0.387 | 1.00 | 22.51 | 7 | N |
| ATOM | 1159 | CA | VAL | A | 152 | 1.767 | 34.677 | 1.533 | 1.00 | 22.20 | 6 | C |
| ATOM | 1160 | C | VAL | A | 152 | 1.980 | 36.123 | 1.961 | 1.00 | 22.68 | 6 | C |
| ATOM | 1161 | O | VAL | A | 152 | 2.470 | 36.950 | 1.201 | 1.00 | 25.11 | 8 | O |
| ATOM | 1162 | CB | VAL | A | 152 | 3.090 | 33.966 | 1.246 | 1.00 | 21.32 | 6 | C |
| ATOM | 1163 | CG1 | VAL | A | 152 | 4.150 | 34.286 | 2.297 | 1.00 | 21.42 | 6 | C |
| ATOM | 1164 | CG2 | VAL | A | 152 | 2.899 | 32.460 | 1.167 | 1.00 | 20.90 | 6 | C |
| ATOM | 1165 | N | TYR | A | 153 | 1.483 | 36.451 | 3.129 | 1.00 | 21.86 | 7 | N |
| ATOM | 1166 | CA | TYR | A | 153 | 1.478 | 37.770 | 3.720 | 1.00 | 21.18 | 6 | C |
| ATOM | 1167 | C | TYR | A | 153 | 2.828 | 38.229 | 4.247 | 1.00 | 21.78 | 6 | C |
| ATOM | 1168 | O | TYR | A | 153 | 3.486 | 37.554 | 5.030 | 1.00 | 22.38 | 8 | O |
| ATOM | 1169 | CB | TYR | A | 153 | 0.471 | 37.751 | 4.877 | 1.00 | 19.74 | 6 | C |
| ATOM | 1170 | CG | TYR | A | 153 | 0.387 | 39.041 | 5.660 | 1.00 | 19.40 | 6 | C |
| ATOM | 1171 | CD1 | TYR | A | 153 | −0.333 | 40.141 | 5.182 | 1.00 | 18.58 | 6 | C |
| ATOM | 1172 | CD2 | TYR | A | 153 | 1.024 | 39.136 | 6.894 | 1.00 | 17.92 | 6 | C |
| ATOM | 1173 | CE1 | TYR | A | 153 | −0.406 | 41.306 | 5.927 | 1.00 | 16.49 | 6 | C |
| ATOM | 1174 | CE2 | TYR | A | 153 | 0.973 | 40.299 | 7.623 | 1.00 | 16.66 | 6 | C |
| ATOM | 1175 | CZ | TYR | A | 153 | 0.247 | 41.387 | 7.137 | 1.00 | 16.66 | 6 | C |
| ATOM | 1176 | OH | TYR | A | 153 | 0.191 | 42.541 | 7.876 | 1.00 | 13.94 | 8 | O |
| ATOM | 1177 | N | TYR | A | 154 | 3.264 | 39.387 | 3.777 | 1.00 | 23.40 | 7 | N |
| ATOM | 1178 | CA | TYR | A | 154 | 4.533 | 39.991 | 4.145 | 1.00 | 24.73 | 6 | C |
| ATOM | 1179 | C | TYR | A | 154 | 4.303 | 41.497 | 4.273 | 1.00 | 24.63 | 6 | C |
| ATOM | 1180 | O | TYR | A | 154 | 3.770 | 42.159 | 3.382 | 1.00 | 22.58 | 8 | O |
| ATOM | 1181 | CB | TYR | A | 154 | 5.693 | 39.698 | 3.182 | 1.00 | 27.54 | 6 | C |
| ATOM | 1182 | CG | TYR | A | 154 | 7.002 | 40.326 | 3.650 | 1.00 | 30.83 | 6 | C |
| ATOM | 1183 | CD1 | TYR | A | 154 | 7.823 | 39.737 | 4.600 | 1.00 | 30.63 | 6 | C |
| ATOM | 1184 | CD2 | TYR | A | 154 | 7.397 | 41.549 | 3.129 | 1.00 | 32.15 | 6 | C |
| ATOM | 1185 | CE1 | TYR | A | 154 | 8.988 | 40.350 | 5.010 | 1.00 | 32.27 | 6 | C |
| ATOM | 1186 | CE2 | TYR | A | 154 | 8.565 | 42.180 | 3.533 | 1.00 | 33.35 | 6 | C |
| ATOM | 1187 | CZ | TYR | A | 154 | 9.352 | 41.570 | 4.488 | 1.00 | 33.35 | 6 | C |
| ATOM | 1188 | OH | TYR | A | 154 | 10.523 | 42.184 | 4.877 | 1.00 | 34.87 | 8 | O |
| ATOM | 1189 | N | GLU | A | 155 | 4.606 | 42.012 | 5.459 | 1.00 | 24.20 | 7 | N |
| ATOM | 1190 | CA | GLU | A | 155 | 4.409 | 43.420 | 5.767 | 1.00 | 23.96 | 6 | C |
| ATOM | 1191 | C | GLU | A | 155 | 5.731 | 44.048 | 6.181 | 1.00 | 24.30 | 6 | C |
| ATOM | 1192 | O | GLU | A | 155 | 6.246 | 43.826 | 7.274 | 1.00 | 23.95 | 8 | O |
| ATOM | 1193 | CB | GLU | A | 155 | 3.366 | 43.568 | 6.876 | 1.00 | 23.12 | 6 | C |
| ATOM | 1194 | CG | GLU | A | 155 | 3.112 | 44.948 | 7.416 | 1.00 | 21.27 | 6 | C |
| ATOM | 1195 | CD | GLU | A | 155 | 2.829 | 46.034 | 6.405 | 1.00 | 21.50 | 6 | C |
| ATOM | 1196 | OE1 | GLU | A | 155 | 1.767 | 46.059 | 5.760 | 1.00 | 18.88 | 8 | O |
| ATOM | 1197 | OE2 | GLU | A | 155 | 3.749 | 46.894 | 6.237 | 1.00 | 23.11 | 8 | O |
| ATOM | 1198 | N | PRO | A | 156 | 6.223 | 44.971 | 5.351 | 1.00 | 25.33 | 7 | N |
| ATOM | 1199 | CA | PRO | A | 156 | 7.471 | 45.663 | 5.590 | 1.00 | 25.20 | 6 | C |
| ATOM | 1200 | C | PRO | A | 156 | 7.529 | 46.411 | 6.911 | 1.00 | 25.30 | 6 | C |
| ATOM | 1201 | O | PRO | A | 156 | 8.606 | 46.479 | 7.516 | 1.00 | 25.86 | 8 | O |
| ATOM | 1202 | CB | PRO | A | 156 | 7.649 | 46.582 | 4.387 | 1.00 | 26.28 | 6 | C |
| ATOM | 1203 | CG | PRO | A | 156 | 6.692 | 46.115 | 3.341 | 1.00 | 25.91 | 6 | C |
| ATOM | 1204 | CD | PRO | A | 156 | 5.616 | 45.330 | 4.039 | 1.00 | 25.57 | 6 | C |
| ATOM | 1205 | N | SER | A | 157 | 6.409 | 46.932 | 7.391 | 1.00 | 24.59 | 7 | N |
| ATOM | 1206 | CA | SER | A | 157 | 6.373 | 47.631 | 8.663 | 1.00 | 24.58 | 6 | C |
| ATOM | 1207 | C | SER | A | 157 | 6.045 | 46.695 | 9.819 | 1.00 | 23.16 | 6 | C |
| ATOM | 1208 | O | SER | A | 157 | 5.767 | 47.224 | 10.895 | 1.00 | 22.52 | 8 | O |
| ATOM | 1209 | CB | SER | A | 157 | 5.364 | 48.790 | 8.606 | 1.00 | 26.25 | 6 | C |
| ATOM | 1210 | OG | SER | A | 157 | 5.761 | 49.711 | 7.603 | 1.00 | 29.54 | 8 | O |
| ATOM | 1211 | N | CYS | A | 158 | 6.107 | 45.371 | 9.637 | 1.00 | 21.33 | 7 | N |
| ATOM | 1212 | CA | CYS | A | 158 | 5.776 | 44.500 | 10.755 | 1.00 | 20.07 | 6 | C |
| ATOM | 1213 | C | CYS | A | 158 | 6.942 | 44.521 | 11.742 | 1.00 | 21.01 | 6 | C |
| ATOM | 1214 | O | CYS | A | 158 | 8.095 | 44.705 | 11.342 | 1.00 | 21.51 | 8 | O |
| ATOM | 1215 | CB | CYS | A | 158 | 5.517 | 43.060 | 10.309 | 1.00 | 16.83 | 6 | C |
| ATOM | 1216 | SG | CYS | A | 158 | 3.927 | 42.529 | 10.989 | 1.00 | 15.02 | 16 | S |
| ATOM | 1217 | N | THR | A | 159 | 6.636 | 44.362 | 13.016 | 1.00 | 21.12 | 7 | N |
| ATOM | 1218 | CA | THR | A | 159 | 7.707 | 44.379 | 14.010 | 1.00 | 21.47 | 6 | C |
| ATOM | 1219 | C | THR | A | 159 | 7.547 | 43.083 | 14.810 | 1.00 | 21.05 | 6 | C |
| ATOM | 1220 | O | THR | A | 159 | 6.509 | 42.456 | 14.662 | 1.00 | 20.59 | 8 | O |
| ATOM | 1221 | CB | THR | A | 159 | 7.643 | 45.484 | 15.066 | 1.00 | 20.41 | 6 | C |
| ATOM | 1222 | OG1 | THR | A | 159 | 6.544 | 45.133 | 15.930 | 1.00 | 19.49 | 8 | O |
| ATOM | 1223 | CG2 | THR | A | 159 | 7.382 | 46.856 | 14.473 | 1.00 | 22.49 | 6 | C |
| ATOM | 1224 | N | GLN | A | 160 | 8.476 | 42.921 | 15.728 | 1.00 | 21.60 | 7 | N |
| ATOM | 1225 | CA | GLN | A | 160 | 8.476 | 41.769 | 16.621 | 1.00 | 22.71 | 6 | C |
| ATOM | 1226 | C | GLN | A | 160 | 7.764 | 41.996 | 17.940 | 1.00 | 20.83 | 6 | C |
| ATOM | 1227 | O | GLN | A | 160 | 7.859 | 41.150 | 18.830 | 1.00 | 19.88 | 8 | O |
| ATOM | 1228 | CB | GLN | A | 160 | 9.944 | 41.372 | 16.841 | 1.00 | 23.85 | 6 | C |
| ATOM | 1229 | CG | GLN | A | 160 | 10.527 | 40.967 | 15.487 | 1.00 | 25.84 | 6 | C |
| ATOM | 1230 | CD | GLN | A | 160 | 11.761 | 40.118 | 15.636 | 1.00 | 27.60 | 6 | C |
| ATOM | 1231 | OE1 | GLN | A | 160 | 11.965 | 39.457 | 16.657 | 1.00 | 29.03 | 8 | O |
| ATOM | 1232 | NE2 | GLN | A | 160 | 12.618 | 40.108 | 14.626 | 1.00 | 27.70 | 7 | N |
| ATOM | 1233 | N | ASN | A | 161 | 7.100 | 43.136 | 18.074 | 1.00 | 19.54 | 7 | N |
| ATOM | 1234 | CA | ASN | A | 161 | 6.282 | 43.407 | 19.257 | 1.00 | 18.99 | 6 | C |
| ATOM | 1235 | C | ASN | A | 161 | 4.952 | 42.685 | 19.015 | 1.00 | 18.67 | 6 | C |
| ATOM | 1236 | O | ASN | A | 161 | 4.067 | 43.319 | 18.420 | 1.00 | 21.11 | 8 | O |
| ATOM | 1237 | CB | ASN | A | 161 | 6.081 | 44.925 | 19.420 | 1.00 | 18.75 | 6 | C |
| ATOM | 1238 | CG | ASN | A | 161 | 7.389 | 45.677 | 19.595 | 1.00 | 20.21 | 6 | C |
| ATOM | 1239 | OD1 | ASN | A | 161 | 7.761 | 46.581 | 18.811 | 1.00 | 22.37 | 8 | O |
| ATOM | 1240 | ND2 | ASN | A | 161 | 8.179 | 45.279 | 20.589 | 1.00 | 15.27 | 7 | N |
| ATOM | 1241 | N | VAL | A | 162 | 4.772 | 41.442 | 19.436 | 1.00 | 16.55 | 7 | N |
| ATOM | 1242 | CA | VAL | A | 162 | 3.538 | 40.724 | 19.200 | 1.00 | 17.16 | 6 | C |
| ATOM | 1243 | C | VAL | A | 162 | 2.366 | 41.269 | 20.013 | 1.00 | 18.41 | 6 | C |
| ATOM | 1244 | O | VAL | A | 162 | 2.507 | 41.695 | 21.176 | 1.00 | 17.26 | 8 | O |
| ATOM | 1245 | CB | VAL | A | 162 | 3.646 | 39.194 | 19.416 | 1.00 | 17.83 | 6 | C |
| ATOM | 1246 | CG1 | VAL | A | 162 | 4.795 | 38.581 | 18.628 | 1.00 | 15.85 | 6 | C |
| ATOM | 1247 | CG2 | VAL | A | 162 | 3.825 | 38.879 | 20.903 | 1.00 | 18.28 | 6 | C |
| ATOM | 1248 | N | ASN | A | 163 | 1.163 | 41.245 | 19.414 | 1.00 | 16.20 | 7 | N |
| ATOM | 1249 | CA | ASN | A | 163 | 0.032 | 41.816 | 20.161 | 1.00 | 16.47 | 6 | C |
| ATOM | 1250 | C | ASN | A | 163 | −1.266 | 41.069 | 19.908 | 1.00 | 16.16 | 6 | C |
| ATOM | 1251 | O | ASN | A | 163 | −2.340 | 41.493 | 20.334 | 1.00 | 17.06 | 8 | O |
| ATOM | 1252 | CB | ASN | A | 163 | −0.121 | 43.297 | 19.770 | 1.00 | 16.08 | 6 | C |
| ATOM | 1253 | CG | ASN | A | 163 | −0.088 | 43.453 | 18.253 | 1.00 | 17.24 | 6 | C |
| ATOM | 1254 | OD1 | ASN | A | 163 | −0.736 | 42.730 | 17.475 | 1.00 | 17.63 | 8 | O |
| ATOM | 1255 | ND2 | ASN | A | 163 | 0.819 | 44.312 | 17.795 | 1.00 | 16.45 | 7 | N |
| ATOM | 1256 | N | HIS | A | 164 | −1.169 | 39.948 | 19.224 | 1.00 | 15.79 | 7 | N |
| ATOM | 1257 | CA | HIS | A | 164 | −2.331 | 39.154 | 18.844 | 1.00 | 15.33 | 6 | C |
| ATOM | 1258 | C | HIS | A | 164 | −1.937 | 37.682 | 18.853 | 1.00 | 15.46 | 6 | C |
| ATOM | 1259 | O | HIS | A | 164 | −0.959 | 37.245 | 18.223 | 1.00 | 15.47 | 8 | O |
| ATOM | 1260 | CB | HIS | A | 164 | −2.839 | 39.624 | 17.468 | 1.00 | 13.97 | 6 | C |
| ATOM | 1261 | CG | HIS | A | 164 | −4.082 | 38.920 | 17.018 | 1.00 | 14.41 | 6 | C |
| ATOM | 1262 | ND1 | HIS | A | 164 | −5.318 | 39.076 | 17.599 | 1.00 | 14.06 | 7 | N |
| ATOM | 1263 | CD2 | HIS | A | 164 | −4.298 | 38.074 | 15.983 | 1.00 | 15.01 | 6 | C |
| ATOM | 1264 | CE1 | HIS | A | 164 | −6.228 | 38.319 | 17.011 | 1.00 | 13.48 | 6 | C |
| ATOM | 1265 | NE2 | HIS | A | 164 | −5.633 | 37.717 | 16.018 | 1.00 | 14.95 | 7 | N |
| ATOM | 1266 | N | GLY | A | 165 | −2.680 | 36.925 | 19.634 | 1.00 | 13.11 | 7 | N |
| ATOM | 1267 | CA | GLY | A | 165 | −2.451 | 35.494 | 19.770 | 1.00 | 13.11 | 6 | C |
| ATOM | 1268 | C | GLY | A | 165 | −3.182 | 34.761 | 18.648 | 1.00 | 13.40 | 6 | C |
| ATOM | 1269 | O | GLY | A | 165 | −4.337 | 35.002 | 18.326 | 1.00 | 11.92 | 8 | O |
| ATOM | 1270 | N | VAL | A | 166 | −2.451 | 33.866 | 18.002 | 1.00 | 12.95 | 7 | N |
| ATOM | 1271 | CA | VAL | A | 166 | −2.949 | 33.066 | 16.886 | 1.00 | 13.37 | 6 | C |
| ATOM | 1272 | C | VAL | A | 156 | −2.417 | 31.640 | 17.048 | 1.00 | 12.90 | 6 | C |
| ATOM | 1273 | O | VAL | A | 166 | −1.705 | 31.346 | 18.023 | 1.00 | 11.55 | 8 | O |
| ATOM | 1274 | CB | VAL | A | 166 | −2.616 | 33.667 | 15.530 | 1.00 | 11.94 | 6 | C |
| ATOM | 1275 | CG1 | VAL | A | 166 | −3.197 | 35.049 | 15.262 | 1.00 | 10.59 | 6 | C |
| ATOM | 1276 | CG2 | VAL | A | 166 | −1.098 | 33.789 | 15.307 | 1.00 | 11.21 | 6 | C |
| ATOM | 1277 | N | LEU | A | 167 | −2.788 | 30.756 | 16.136 | 1.00 | 13.69 | 7 | N |
| ATOM | 1278 | CA | LEU | A | 167 | −2.414 | 29.349 | 16.235 | 1.00 | 14.04 | 6 | C |
| ATOM | 1279 | C | LEU | A | 167 | −1.733 | 28.906 | 14.938 | 1.00 | 14.23 | 6 | C |
| ATOM | 1280 | O | LEU | A | 167 | −2.333 | 28.991 | 13.880 | 1.00 | 14.35 | 8 | O |
| ATOM | 1281 | CB | LEU | A | 167 | −3.627 | 28.460 | 16.429 | 1.00 | 14.60 | 6 | C |
| ATOM | 1282 | CG | LEU | A | 167 | −3.676 | 27.221 | 17.318 | 1.00 | 18.08 | 6 | C |
| ATOM | 1283 | CD1 | LEU | A | 167 | −4.331 | 26.042 | 16.608 | 1.00 | 15.69 | 6 | C |
| ATOM | 1284 | CD2 | LEU | A | 167 | −2.382 | 26.803 | 17.986 | 1.00 | 16.33 | 6 | C |
| ATOM | 1285 | N | VAL | A | 168 | −0.509 | 28.432 | 15.013 | 1.00 | 15.60 | 7 | N |
| ATOM | 1286 | CA | VAL | A | 168 | 0.193 | 27.945 | 13.827 | 1.00 | 14.54 | 6 | C |
| ATOM | 1287 | C | VAL | A | 168 | −0.213 | 26.472 | 13.689 | 1.00 | 16.18 | 6 | C |
| ATOM | 1288 | O | VAL | A | 168 | 0.168 | 25.658 | 14.535 | 1.00 | 14.48 | 8 | O |
| ATOM | 1289 | CB | VAL | A | 168 | 1.712 | 28.056 | 13.929 | 1.00 | 13.43 | 6 | C |
| ATOM | 1290 | CG1 | VAL | A | 168 | 2.350 | 27.371 | 12.717 | 1.00 | 14.46 | 6 | C |
| ATOM | 1291 | CG2 | VAL | A | 168 | 2.155 | 29.515 | 14.012 | 1.00 | 12.07 | 6 | C |
| ATOM | 1292 | N | VAL | A | 169 | −1.051 | 26.187 | 12.685 | 1.00 | 15.86 | 7 | N |
| ATOM | 1293 | CA | VAL | A | 169 | −1.555 | 24.836 | 12.461 | 1.00 | 15.27 | 6 | C |
| ATOM | 1294 | C | VAL | A | 169 | −0.798 | 24.132 | 11.345 | 1.00 | 16.90 | 6 | C |
| ATOM | 1295 | O | VAL | A | 169 | −0.951 | 22.931 | 11.102 | 1.00 | 16.96 | 8 | O |
| ATOM | 1296 | CB | VAL | A | 169 | −3.063 | 24.818 | 12.161 | 1.00 | 14.63 | 6 | C |
| ATOM | 1297 | CG1 | VAL | A | 169 | −3.850 | 25.372 | 13.345 | 1.00 | 13.34 | 6 | C |
| ATOM | 1298 | CG2 | VAL | A | 169 | −3.398 | 25.625 | 10.917 | 1.00 | 12.70 | 6 | C |
| ATOM | 1299 | N | GLY | A | 170 | 0.096 | 24.865 | 10.665 | 1.00 | 17.95 | 7 | N |
| ATOM | 1300 | CA | GLY | A | 170 | 1.017 | 24.211 | 9.750 | 1.00 | 17.61 | 6 | C |
| ATOM | 1301 | C | GLY | A | 170 | 2.018 | 25.147 | 9.088 | 1.00 | 17.35 | 6 | C |
| ATOM | 1302 | O | GLY | A | 170 | 2.433 | 26.169 | 9.619 | 1.00 | 16.20 | 8 | O |
| ATOM | 1303 | N | TYR | A | 171 | 2.632 | 24.564 | 8.037 | 1.00 | 17.22 | 7 | N |
| ATOM | 1304 | CA | TYR | A | 171 | 3.548 | 25.356 | 7.218 | 1.00 | 17.14 | 6 | C |
| ATOM | 1305 | C | TYR | A | 171 | 3.652 | 24.784 | 5.807 | 1.00 | 17.19 | 6 | C |
| ATOM | 1306 | O | TYR | A | 171 | 3.243 | 23.658 | 5.546 | 1.00 | 14.93 | 8 | O |
| ATOM | 1307 | CB | TYR | A | 171 | 4.919 | 25.455 | 7.876 | 1.00 | 16.29 | 6 | C |
| ATOM | 1308 | CG | TYR | A | 171 | 5.615 | 24.125 | 8.120 | 1.00 | 17.70 | 6 | C |
| ATOM | 1309 | CD1 | TYR | A | 171 | 6.289 | 23.446 | 7.108 | 1.00 | 16.54 | 6 | C |
| ATOM | 1310 | CD2 | TYR | A | 171 | 5.607 | 23.550 | 9.386 | 1.00 | 17.49 | 6 | C |
| ATOM | 1311 | CE1 | TYR | A | 171 | 6.908 | 22.231 | 7.369 | 1.00 | 17.16 | 6 | C |
| ATOM | 1312 | CE2 | TYR | A | 171 | 6.237 | 22.348 | 9.636 | 1.00 | 17.68 | 6 | C |
| ATOM | 1313 | CZ | TYR | A | 171 | 6.897 | 21.679 | 8.627 | 1.00 | 17.44 | 6 | C |
| ATOM | 1314 | OH | TYR | A | 171 | 7.538 | 20.473 | 8.845 | 1.00 | 18.24 | 8 | O |
| ATOM | 1315 | N | GLY | A | 172 | 4.200 | 25.574 | 4.892 | 1.00 | 17.34 | 7 | N |
| ATOM | 1316 | CA | GLY | A | 172 | 4.458 | 25.038 | 3.556 | 1.00 | 21.15 | 6 | C |
| ATOM | 1317 | C | GLY | A | 172 | 5.181 | 26.092 | 2.731 | 1.00 | 24.74 | 6 | C |
| ATOM | 1318 | O | GLY | A | 172 | 5.864 | 27.013 | 3.201 | 1.00 | 22.56 | 8 | O |
| ATOM | 1319 | N | ASP | A | 173 | 4.764 | 26.121 | 1.471 | 1.00 | 27.84 | 7 | N |
| ATOM | 1320 | CA | ASP | A | 173 | 5.390 | 26.992 | 0.484 | 1.00 | 32.33 | 6 | C |
| ATOM | 1321 | C | ASP | A | 173 | 4.450 | 27.197 | −0.694 | 1.00 | 33.93 | 6 | C |
| ATOM | 1322 | O | ASP | A | 173 | 3.875 | 26.262 | −1.245 | 1.00 | 33.57 | 8 | O |
| ATOM | 1323 | CB | ASP | A | 173 | 6.726 | 26.322 | 0.152 | 1.00 | 35.77 | 6 | C |
| ATOM | 1324 | CG | ASP | A | 173 | 7.031 | 26.392 | −1.330 | 1.00 | 37.36 | 6 | C |
| ATOM | 1325 | OD1 | ASP | A | 173 | 6.536 | 25.496 | −2.041 | 1.00 | 36.96 | 8 | O |
| ATOM | 1326 | OD2 | ASP | A | 173 | 7.695 | 27.373 | −1.708 | 1.00 | 39.64 | 8 | O |
| ATOM | 1327 | N | LEU | A | 174 | 4.254 | 28.459 | −1.018 | 1.00 | 35.00 | 7 | N |
| ATOM | 1328 | CA | LEU | A | 174 | 3.399 | 28.910 | −2.100 | 1.00 | 38.08 | 6 | C |
| ATOM | 1329 | C | LEU | A | 174 | 4.252 | 29.425 | −3.259 | 1.00 | 39.09 | 6 | C |
| ATOM | 1330 | O | LEU | A | 174 | 4.669 | 30.585 | −3.221 | 1.00 | 38.29 | 8 | O |
| ATOM | 1331 | CB | LEU | A | 174 | 2.511 | 30.040 | −1.555 | 1.00 | 38.22 | 6 | C |
| ATOM | 1332 | CG | LEU | A | 174 | 1.173 | 30.291 | −2.237 | 1.00 | 39.46 | 6 | C |
| ATOM | 1333 | CD1 | LEU | A | 174 | 0.332 | 31.322 | −1.495 | 1.00 | 39.18 | 6 | C |
| ATOM | 1334 | CD2 | LEU | A | 174 | 1.355 | 30.748 | −3.680 | 1.00 | 39.84 | 6 | C |
| ATOM | 1335 | N | ASN | A | 175 | 4.624 | 28.562 | −4.195 | 1.00 | 40.67 | 7 | N |
| ATOM | 1336 | CA | ASN | A | 175 | 5.408 | 28.947 | −5.366 | 1.00 | 42.16 | 6 | C |
| ATOM | 1337 | C | ASN | A | 175 | 6.697 | 29.663 | −4.988 | 1.00 | 42.41 | 6 | C |
| ATOM | 1338 | O | ASN | A | 175 | 6.964 | 30.829 | −5.284 | 1.00 | 43.04 | 8 | O |
| ATOM | 1339 | CB | ASN | A | 175 | 4.560 | 29.804 | −6.309 | 1.00 | 43.20 | 6 | C |
| ATOM | 1340 | CG | ASN | A | 175 | 3.397 | 29.033 | −6.912 | 1.00 | 44.56 | 6 | C |
| ATOM | 1341 | GD1 | ASN | A | 175 | 2.267 | 29.523 | −6.853 | 1.00 | 44.33 | 8 | O |
| ATOM | 1342 | ND2 | ASN | A | 175 | 3.705 | 27.865 | −7.477 | 1.00 | 45.34 | 7 | N |
| ATOM | 1343 | N | GLY | A | 176 | 7.517 | 28.980 | −4.192 | 1.00 | 41.86 | 7 | N |
| ATOM | 1344 | CA | GLY | A | 176 | 8.739 | 29.541 | −3.652 | 1.00 | 40.44 | 6 | C |
| ATOM | 1345 | C | GLY | A | 176 | 8.591 | 30.376 | −2.390 | 1.00 | 38.18 | 6 | C |
| ATOM | 1346 | O | GLY | A | 176 | 9.626 | 30.654 | −1.773 | 1.00 | 38.68 | 8 | O |
| ATOM | 1347 | N | LYS | A | 177 | 7.394 | 30.755 | −1.962 | 1.00 | 35.10 | 7 | N |
| ATOM | 1348 | CA | LYS | A | 177 | 7.269 | 31.573 | −0.761 | 1.00 | 32.21 | 6 | C |
| ATOM | 1349 | C | LYS | A | 177 | 6.771 | 30.754 | 0.436 | 1.00 | 28.29 | 6 | C |
| ATOM | 1350 | O | LYS | A | 177 | 5.615 | 30.433 | 0.670 | 1.00 | 27.65 | 8 | O |
| ATOM | 1351 | CB | LYS | A | 177 | 6.450 | 32.837 | −0.969 | 1.00 | 33.72 | 6 | C |
| ATOM | 1352 | CG | LYS | A | 177 | 6.833 | 33.757 | −2.108 | 1.00 | 35.81 | 6 | C |
| ATOM | 1353 | CD | LYS | A | 177 | 7.591 | 35.016 | −1.754 | 1.00 | 37.22 | 6 | C |
| ATOM | 1354 | CE | LYS | A | 177 | 9.097 | 34.886 | −1.745 | 1.00 | 39.10 | 6 | C |
| ATOM | 1355 | NZ | LYS | A | 177 | 9.789 | 36.044 | −2.400 | 1.00 | 39.84 | 7 | N |
| ATOM | 1356 | N | GLU | A | 178 | 7.759 | 30.472 | 1.286 | 1.00 | 24.46 | 7 | N |
| ATOM | 1357 | CA | GLU | A | 178 | 7.610 | 29.784 | 2.546 | 1.00 | 22.50 | 6 | C |
| ATOM | 1358 | C | GLU | A | 178 | 6.635 | 30.529 | 3.455 | 1.00 | 18.46 | 6 | C |
| ATOM | 1359 | O | GLU | A | 178 | 6.736 | 31.726 | 3.604 | 1.00 | 15.36 | 8 | O |
| ATOM | 1360 | CB | GLU | A | 178 | 8.959 | 29.601 | 3.267 | 1.00 | 24.33 | 6 | C |
| ATOM | 1361 | CG | GLU | A | 178 | 9.930 | 28.749 | 2.453 | 1.00 | 26.00 | 6 | C |
| ATOM | 1362 | CD | GLU | A | 178 | 11.076 | 28.212 | 3.286 | 1.00 | 27.06 | 6 | C |
| ATOM | 1363 | OE1 | GLU | A | 178 | 11.434 | 28.854 | 4.287 | 1.00 | 27.85 | 8 | O |
| ATOM | 1364 | OE2 | GLU | A | 178 | 11.606 | 27.130 | 2.955 | 1.00 | 28.93 | 8 | O |
| ATOM | 1365 | N | TYR | A | 179 | 5.672 | 29.836 | 4.032 | 1.00 | 17.29 | 7 | N |
| ATOM | 1366 | CA | TYR | A | 179 | 4.774 | 30.471 | 4.988 | 1.00 | 17.64 | 6 | C |
| ATOM | 1367 | C | TYR | A | 179 | 4.516 | 29.592 | 6.207 | 1.00 | 15.62 | 6 | C |
| ATOM | 1368 | O | TYR | A | 179 | 4.794 | 28.402 | 6.176 | 1.00 | 13.21 | 8 | O |
| ATOM | 1369 | CB | TYR | A | 179 | 3.411 | 30.758 | 4.327 | 1.00 | 18.27 | 6 | C |
| ATOM | 1370 | CG | TYR | A | 179 | 2.738 | 29.471 | 3.876 | 1.00 | 20.52 | 6 | C |
| ATOM | 1371 | CD1 | TYR | A | 179 | 2.018 | 28.698 | 4.767 | 1.00 | 20.72 | 6 | C |
| ATOM | 1372 | CD2 | TYR | A | 179 | 2.756 | 29.079 | 2.543 | 1.00 | 21.28 | 6 | C |
| ATOM | 1373 | CE1 | TYR | A | 179 | 1.381 | 27.533 | 4.379 | 1.00 | 22.12 | 6 | C |
| ATOM | 1374 | CE2 | TYR | A | 179 | 2.104 | 27.931 | 2.133 | 1.00 | 22.09 | 6 | C |
| ATOM | 1375 | CZ | TYR | A | 179 | 1.435 | 27.152 | 3.051 | 1.00 | 22.22 | 6 | C |
| ATOM | 1376 | OH | TYR | A | 179 | 0.798 | 26.008 | 2.646 | 1.00 | 21.39 | 8 | O |
| ATOM | 1377 | N | TRP | A | 180 | 3.854 | 30.207 | 7.170 | 1.00 | 15.61 | 7 | N |
| ATOM | 1378 | CA | TRP | A | 180 | 3.295 | 29.578 | 8.353 | 1.00 | 16.38 | 6 | C |
| ATOM | 1379 | C | TRP | A | 180 | 1.762 | 29.623 | 8.235 | 1.00 | 16.72 | 6 | C |
| ATOM | 1380 | O | TRP | A | 180 | 1.215 | 30.720 | 8.033 | 1.00 | 16.17 | 8 | O |
| ATOM | 1381 | CB | TRP | A | 180 | 3.656 | 30.415 | 9.594 | 1.00 | 15.46 | 6 | C |
| ATOM | 1382 | CG | TRP | A | 180 | 5.097 | 30.294 | 9.994 | 1.00 | 16.15 | 6 | C |
| ATOM | 1383 | CD1 | TRP | A | 180 | 6.022 | 31.306 | 9.967 | 1.00 | 15.97 | 6 | C |
| ATOM | 1384 | CD2 | TRP | A | 180 | 5.796 | 29.136 | 10.482 | 1.00 | 16.39 | 6 | C |
| ATOM | 1385 | NE1 | TRP | A | 180 | 7.238 | 30.855 | 10.414 | 1.00 | 15.50 | 7 | N |
| ATOM | 1386 | CE2 | TRP | A | 180 | 7.130 | 29.529 | 10.744 | 1.00 | 16.18 | 6 | C |
| ATOM | 1387 | CE3 | TRP | A | 180 | 5.432 | 27.808 | 10.735 | 1.00 | 15.39 | 6 | C |
| ATOM | 1388 | CZ2 | TRP | A | 180 | 8.099 | 28.639 | 11.222 | 1.00 | 16.42 | 6 | C |
| ATOM | 1389 | CZ3 | TRP | A | 180 | 6.380 | 26.946 | 11.239 | 1.00 | 14.76 | 6 | C |
| ATOM | 1390 | CH2 | TRP | A | 180 | 7.709 | 27.355 | 11.477 | 1.00 | 15.13 | 6 | C |
| ATOM | 1391 | N | LEU | A | 181 | 1.065 | 28.500 | 8.305 | 1.00 | 16.97 | 7 | N |
| ATOM | 1392 | CA | LEU | A | 181 | −0.406 | 28.491 | 8.193 | 1.00 | 14.53 | 6 | C |
| ATOM | 1393 | C | LEU | A | 181 | −1.017 | 28.881 | 9.526 | 1.00 | 15.04 | 6 | C |
| ATOM | 1394 | O | LEU | A | 181 | −0.865 | 28.155 | 10.519 | 1.00 | 16.01 | 8 | O |
| ATOM | 1395 | CB | LEU | A | 181 | −0.801 | 27.060 | 7.761 | 1.00 | 12.54 | 6 | C |
| ATOM | 1396 | CG | LEU | A | 181 | −2.292 | 26.806 | 7.526 | 1.00 | 12.47 | 6 | C |
| ATOM | 1397 | CD1 | LEU | A | 181 | −2.885 | 27.752 | 6.494 | 1.00 | 10.12 | 6 | C |
| ATOM | 1398 | CD2 | LEU | A | 181 | −2.572 | 25.353 | 7.146 | 1.00 | 13.57 | 6 | C |
| ATOM | 1399 | N | VAL | A | 182 | −1.619 | 30.066 | 9.599 | 1.00 | 15.67 | 7 | N |
| ATOM | 1400 | CA | VAL | A | 182 | −2.136 | 30.572 | 10.874 | 1.00 | 15.34 | 6 | C |
| ATOM | 1401 | C | VAL | A | 182 | −3.657 | 30.555 | 10.944 | 1.00 | 16.57 | 6 | C |
| ATOM | 1402 | O | VAL | A | 182 | −4.366 | 30.998 | 10.033 | 1.00 | 15.01 | 8 | O |
| ATOM | 1403 | CB | VAL | A | 182 | −1.661 | 32.039 | 11.041 | 1.00 | 15.50 | 6 | C |
| ATOM | 1404 | CG1 | VAL | A | 182 | −2.086 | 32.625 | 12.368 | 1.00 | 13.96 | 6 | C |
| ATOM | 1405 | CG2 | VAL | A | 182 | −0.148 | 32.108 | 10.852 | 1.00 | 14.73 | 6 | C |
| ATOM | 1406 | N | LYS | A | 183 | −4.178 | 29.969 | 12.012 | 1.00 | 15.60 | 7 | N |
| ATOM | 1407 | CA | LYS | A | 183 | −5.590 | 29.975 | 12.323 | 1.00 | 15.18 | 6 | C |
| ATOM | 1408 | C | LYS | A | 183 | −5.825 | 31.243 | 13.158 | 1.00 | 14.69 | 6 | C |
| ATOM | 1409 | O | LYS | A | 183 | −5.159 | 31.432 | 14.178 | 1.00 | 13.60 | 8 | O |
| ATOM | 1410 | CB | LYS | A | 183 | −6.002 | 28.761 | 13.154 | 1.00 | 14.92 | 6 | C |
| ATOM | 1411 | CG | LYS | A | 183 | −7.505 | 28.610 | 13.307 | 1.00 | 14.25 | 6 | C |
| ATOM | 1412 | CD | LYS | A | 183 | −7.826 | 27.594 | 14.403 | 1.00 | 15.02 | 6 | C |
| ATOM | 1413 | CE | LYS | A | 183 | −9.333 | 27.333 | 14.442 | 1.00 | 15.19 | 6 | C |
| ATOM | 1414 | NZ | LYS | A | 183 | −9.781 | 26.510 | 15.594 | 1.00 | 16.20 | 7 | N |
| ATOM | 1415 | N | ASN | A | 184 | −6.689 | 32.119 | 12.701 | 1.00 | 13.87 | 7 | N |
| ATOM | 1416 | CA | ASN | A | 184 | −6.997 | 33.342 | 13.440 | 1.00 | 13.73 | 6 | C |
| ATOM | 1417 | C | ASN | A | 184 | −8.281 | 33.107 | 14.235 | 1.00 | 13.70 | 6 | C |
| ATOM | 1418 | O | ASN | A | 184 | −8.895 | 32.056 | 14.053 | 1.00 | 12.97 | 8 | O |
| ATOM | 1419 | CB | ASN | A | 184 | −7.185 | 34.510 | 12.488 | 1.00 | 12.85 | 6 | C |
| ATOM | 1420 | CG | ASN | A | 184 | −7.039 | 35.876 | 13.128 | 1.00 | 14.41 | 6 | C |
| ATOM | 1421 | OD1 | ASN | A | 184 | −6.754 | 36.061 | 14.305 | 1.00 | 14.25 | 8 | O |
| ATOM | 1422 | ND2 | ASN | A | 184 | −7.162 | 36.922 | 12.330 | 1.00 | 13.86 | 7 | N |
| ATOM | 1423 | N | SER | A | 185 | −8.782 | 34.116 | 14.933 | 1.00 | 14.88 | 7 | N |
| ATOM | 1424 | CA | SER | A | 185 | −10.053 | 33.976 | 15.671 | 1.00 | 15.71 | 6 | C |
| ATOM | 1425 | C | SER | A | 185 | −10.905 | 35.173 | 15.278 | 1.00 | 17.28 | 6 | C |
| ATOM | 1426 | O | SER | A | 185 | −11.582 | 35.831 | 16.061 | 1.00 | 18.08 | 8 | O |
| ATOM | 1427 | CB | SER | A | 185 | −9.754 | 33.878 | 17.165 | 1.00 | 13.97 | 6 | C |
| ATOM | 1428 | OG | SER | A | 185 | −8.880 | 34.956 | 17.544 | 1.00 | 14.91 | 8 | O |
| ATOM | 1429 | N | TRP | A | 186 | −10.900 | 35.480 | 13.975 | 1.00 | 18.64 | 7 | N |
| ATOM | 1430 | CA | TRP | A | 186 | −11.626 | 36.637 | 13.444 | 1.00 | 19.40 | 6 | C |
| ATOM | 1431 | C | TRP | A | 186 | −12.789 | 36.200 | 12.583 | 1.00 | 19.87 | 6 | C |
| ATOM | 1432 | O | TRP | A | 186 | −13.275 | 36.935 | 11.717 | 1.00 | 19.64 | 8 | O |
| ATOM | 1433 | CB | TRP | A | 186 | −10.676 | 37.599 | 12.701 | 1.00 | 19.39 | 6 | C |
| ATOM | 1434 | CG | TRP | A | 186 | −9.884 | 38.499 | 13.617 | 1.00 | 21.79 | 6 | C |
| ATOM | 1435 | CD1 | TRP | A | 186 | −9.854 | 38.479 | 14.984 | 1.00 | 21.06 | 6 | C |
| ATOM | 1436 | CD2 | TRP | A | 186 | −8.908 | 39.484 | 13.230 | 1.00 | 21.63 | 6 | C |
| ATOM | 1437 | NE1 | TRP | A | 186 | −8.986 | 39.408 | 15.470 | 1.00 | 21.86 | 7 | N |
| ATOM | 1438 | CE2 | TRP | A | 186 | −8.351 | 40.010 | 14.412 | 1.00 | 21.21 | 6 | C |
| ATOM | 1439 | CE3 | TRP | A | 186 | −8.459 | 39.966 | 11.996 | 1.00 | 21.18 | 6 | C |
| ATOM | 1440 | CZ2 | TRP | A | 186 | −7.435 | 41.055 | 14.403 | 1.00 | 20.81 | 6 | C |
| ATOM | 1441 | CZ3 | TRP | A | 186 | −7.532 | 41.016 | 12.000 | 1.00 | 21.65 | 6 | C |
| ATOM | 1442 | CH2 | TRP | A | 186 | −7.034 | 41.552 | 13.197 | 1.00 | 19.62 | 6 | C |
| ATOM | 1443 | N | GLY | A | 187 | −13.290 | 34.987 | 12.823 | 1.00 | 20.15 | 7 | N |
| ATOM | 1444 | CA | GLY | A | 187 | −14.435 | 34.493 | 12.074 | 1.00 | 20.66 | 6 | C |
| ATOM | 1445 | C | GLY | A | 187 | −13.985 | 33.932 | 10.718 | 1.00 | 23.32 | 6 | C |
| ATOM | 1446 | O | GLY | A | 187 | −12.874 | 34.115 | 10.234 | 1.00 | 21.54 | 8 | O |
| ATOM | 1447 | N | HIS | A | 188 | −14.955 | 33.341 | 10.023 | 1.00 | 25.27 | 7 | N |
| ATOM | 1448 | CA | HIS | A | 188 | −14.684 | 32.693 | 8.745 | 1.00 | 28.37 | 6 | C |
| ATOM | 1449 | C | HIS | A | 188 | −14.552 | 33.705 | 7.622 | 1.00 | 26.86 | 6 | C |
| ATOM | 1450 | O | HIS | A | 188 | −13.845 | 33.388 | 6.653 | 1.00 | 25.55 | 8 | O |
| ATOM | 1451 | CB | HIS | A | 188 | −15.671 | 31.574 | 8.493 | 1.00 | 32.95 | 6 | C |
| ATOM | 1452 | CG | HIS | A | 188 | −16.996 | 31.794 | 7.859 | 1.00 | 37.55 | 6 | C |
| ATOM | 1453 | ND1 | HIS | A | 188 | −17.623 | 30.760 | 7.178 | 1.00 | 39.31 | 7 | N |
| ATOM | 1454 | CD2 | HIS | A | 188 | −17.827 | 32.871 | 7.766 | 1.00 | 38.66 | 6 | C |
| ATOM | 1455 | CE1 | HIS | A | 188 | −18.786 | 31.184 | 6.704 | 1.00 | 39.66 | 6 | C |
| ATOM | 1456 | NE2 | HIS | A | 188 | −18.925 | 32.455 | 7.052 | 1.00 | 39.70 | 7 | N |
| ATOM | 1457 | N | ASN | A | 189 | −14.994 | 34.947 | 7.757 | 1.00 | 23.83 | 7 | N |
| ATOM | 1458 | CA | ASN | A | 189 | −14.835 | 35.913 | 6.676 | 1.00 | 23.09 | 6 | C |
| ATOM | 1459 | C | ASN | A | 189 | −13.506 | 36.657 | 6.649 | 1.00 | 23.00 | 6 | C |
| ATOM | 1460 | O | ASN | A | 189 | −13.137 | 37.361 | 5.695 | 1.00 | 21.38 | 8 | O |
| ATOM | 1461 | CB | ASN | A | 189 | −16.032 | 36.876 | 6.651 | 1.00 | 24.25 | 6 | C |
| ATOM | 1462 | CG | ASN | A | 189 | −17.300 | 36.052 | 6.434 | 1.00 | 24.98 | 6 | C |
| ATOM | 1463 | OD1 | ASN | A | 189 | −18.237 | 36.112 | 7.221 | 1.00 | 27.48 | 8 | O |
| ATOM | 1464 | ND2 | ASN | A | 189 | −17.256 | 35.174 | 5.446 | 1.00 | 26.05 | 7 | N |
| ATOM | 1465 | N | PHE | A | 190 | −12.643 | 36.387 | 7.618 | 1.00 | 20.07 | 7 | N |
| ATOM | 1466 | CA | PHE | A | 190 | −11.257 | 36.828 | 7.584 | 1.00 | 17.77 | 6 | C |
| ATOM | 1467 | C | PHE | A | 190 | −10.417 | 35.944 | 6.656 | 1.00 | 17.53 | 6 | C |
| ATOM | 1468 | O | PHE | A | 190 | −10.494 | 34.705 | 6.594 | 1.00 | 17.41 | 8 | O |
| ATOM | 1469 | CB | PHE | A | 190 | −10.671 | 36.851 | 9.003 | 1.00 | 16.47 | 6 | C |
| ATOM | 1470 | CG | PHE | A | 190 | −9.182 | 37.069 | 8.988 | 1.00 | 14.40 | 6 | C |
| ATOM | 1471 | CD1 | PHE | A | 190 | −8.318 | 35.989 | 9.188 | 1.00 | 14.66 | 6 | C |
| ATOM | 1472 | CD2 | PHE | A | 190 | −8.643 | 38.312 | 8.734 | 1.00 | 14.20 | 6 | C |
| ATOM | 1473 | CE1 | PHE | A | 190 | −6.954 | 36.181 | 9.151 | 1.00 | 15.22 | 6 | C |
| ATOM | 1474 | CE2 | PHE | A | 190 | −7.276 | 38.501 | 8.700 | 1.00 | 13.98 | 6 | C |
| ATOM | 1475 | CZ | PHE | A | 190 | −6.419 | 37.424 | 8.907 | 1.00 | 12.99 | 6 | C |
| ATOM | 1476 | N | GLY | A | 191 | −9.553 | 36.582 | 5.863 | 1.00 | 15.91 | 7 | N |
| ATOM | 1477 | CA | GLY | A | 191 | −8.573 | 35.919 | 5.026 | 1.00 | 16.14 | 6 | C |
| ATOM | 1478 | C | GLY | A | 191 | −9.087 | 34.693 | 4.293 | 1.00 | 18.11 | 6 | C |
| ATOM | 1479 | O | GLY | A | 191 | −10.176 | 34.779 | 3.719 | 1.00 | 18.26 | 8 | O |
| ATOM | 1480 | N | GLU | A | 192 | −8.346 | 33.578 | 4.320 | 1.00 | 17.12 | 7 | N |
| ATOM | 1481 | CA | GLU | A | 192 | −8.754 | 32.368 | 3.635 | 1.00 | 17.98 | 6 | C |
| ATOM | 1482 | C | GLU | A | 192 | −9.560 | 31.470 | 4.565 | 1.00 | 17.03 | 6 | C |
| ATOM | 1483 | O | GLU | A | 192 | −9.028 | 30.609 | 5.276 | 1.00 | 16.42 | 8 | O |
| ATOM | 1484 | CB | GLU | A | 192 | −7.514 | 31.629 | 3.080 | 1.00 | 19.96 | 6 | C |
| ATOM | 1485 | CG | GLU | A | 192 | −6.641 | 32.515 | 2.214 | 1.00 | 23.37 | 6 | C |
| ATOM | 1486 | CD | GLU | A | 192 | −5.267 | 31.975 | 1.891 | 1.00 | 26.37 | 6 | C |
| ATOM | 1487 | OE1 | GLU | A | 192 | −4.557 | 31.515 | 2.811 | 1.00 | 26.96 | 8 | O |
| ATOM | 1488 | OE2 | GLU | A | 192 | −4.882 | 32.006 | 0.698 | 1.00 | 28.12 | 8 | O |
| ATOM | 1489 | N | GLU | A | 193 | −10.860 | 31.723 | 4.656 | 1.00 | 15.32 | 7 | N |
| ATOM | 1490 | CA | GLU | A | 193 | −11.773 | 30.999 | 5.521 | 1.00 | 17.56 | 6 | C |
| ATOM | 1491 | C | GLU | A | 193 | −11.334 | 31.111 | 6.981 | 1.00 | 17.17 | 6 | C |
| ATOM | 1492 | O | GLU | A | 193 | −11.379 | 30.116 | 7.716 | 1.00 | 17.09 | 8 | O |
| ATOM | 1493 | CB | GLU | A | 193 | −11.892 | 29.512 | 5.127 | 1.00 | 18.86 | 6 | C |
| ATOM | 1494 | CG | GLU | A | 193 | −12.376 | 29.242 | 3.702 | 1.00 | 23.04 | 6 | C |
| ATOM | 1495 | CD | GLU | A | 193 | −12.734 | 27.783 | 3.435 | 1.00 | 24.86 | 6 | C |
| ATOM | 1496 | OE1 | GLU | A | 193 | −13.411 | 27.186 | 4.307 | 1.00 | 27.13 | 8 | O |
| ATOM | 1497 | OE2 | GLU | A | 193 | −12.396 | 27.184 | 2.397 | 1.00 | 24.55 | 8 | O |
| ATOM | 1498 | N | GLY | A | 194 | −10.738 | 32.219 | 7.391 | 1.00 | 17.11 | 7 | N |
| ATOM | 1499 | CA | GLY | A | 194 | −10.314 | 32.452 | 8.757 | 1.00 | 16.49 | 6 | C |
| ATOM | 1500 | C | GLY | A | 194 | −8.823 | 32.203 | 8.994 | 1.00 | 16.49 | 6 | C |
| ATOM | 1501 | O | GLY | A | 194 | −8.367 | 32.444 | 10.114 | 1.00 | 14.95 | 8 | O |
| ATOM | 1502 | N | TYR | A | 195 | −8.120 | 31.825 | 7.942 | 1.00 | 16.25 | 7 | N |
| ATOM | 1503 | CA | TYR | A | 195 | −6.696 | 31.552 | 8.020 | 1.00 | 17.48 | 6 | C |
| ATOM | 1504 | C | TYR | A | 195 | −5.877 | 32.624 | 7.294 | 1.00 | 17.17 | 6 | C |
| ATOM | 1505 | O | TYR | A | 195 | −6.351 | 33.300 | 6.387 | 1.00 | 16.71 | 8 | O |
| ATOM | 1506 | CB | TYR | A | 195 | −6.320 | 30.170 | 7.434 | 1.00 | 17.38 | 6 | C |
| ATOM | 1507 | CG | TYR | A | 195 | −6.815 | 29.046 | 8.326 | 1.00 | 17.46 | 6 | C |
| ATOM | 1508 | CD1 | TYR | A | 195 | −8.172 | 28.719 | 8.324 | 1.00 | 19.12 | 6 | C |
| ATOM | 1509 | CD2 | TYR | A | 195 | −5.956 | 28.397 | 9.193 | 1.00 | 17.98 | 6 | C |
| ATOM | 1510 | CE1 | TYR | A | 195 | −8.623 | 27.702 | 9.152 | 1.00 | 19.16 | 6 | C |
| ATOM | 1511 | CE2 | TYR | A | 195 | −6.431 | 27.433 | 10.047 | 1.00 | 17.77 | 6 | C |
| ATOM | 1512 | CZ | TYR | A | 195 | −7.745 | 27.054 | 10.000 | 1.00 | 19.28 | 6 | C |
| ATOM | 1513 | OH | TYR | A | 195 | −8.230 | 26.069 | 10.839 | 1.00 | 19.78 | 8 | O |
| ATOM | 1514 | N | ILE | A | 196 | −4.607 | 32.654 | 7.664 | 1.00 | 15.81 | 7 | N |
| ATOM | 1515 | CA | ILE | A | 196 | −3.646 | 33.523 | 7.003 | 1.00 | 16.14 | 6 | C |
| ATOM | 1516 | C | ILE | A | 196 | −2.317 | 32.805 | 6.878 | 1.00 | 16.66 | 6 | C |
| ATOM | 1517 | O | ILE | A | 196 | −1.834 | 32.123 | 7.789 | 1.00 | 19.33 | 8 | O |
| ATOM | 1518 | CB | ILE | A | 196 | −3.559 | 34.904 | 7.682 | 1.00 | 15.06 | 6 | C |
| ATOM | 1519 | CG1 | ILE | A | 196 | −2.544 | 35.842 | 7.026 | 1.00 | 14.70 | 6 | C |
| ATOM | 1520 | CG2 | ILE | A | 196 | −3.252 | 34.738 | 9.167 | 1.00 | 14.78 | 6 | C |
| ATOM | 1521 | GD1 | ILE | A | 196 | −2.527 | 37.257 | 7.562 | 1.00 | 14.90 | 6 | C |
| ATOM | 1522 | N | ARG | A | 197 | −1.688 | 32.948 | 5.725 | 1.00 | 18.43 | 7 | N |
| ATOM | 1523 | CA | ARG | A | 197 | −0.371 | 32.360 | 5.458 | 1.00 | 19.44 | 6 | C |
| ATOM | 1524 | C | ARG | A | 197 | 0.655 | 33.508 | 5.513 | 1.00 | 18.85 | 6 | C |
| ATOM | 1525 | O | ARG | A | 197 | 0.558 | 34.487 | 4.751 | 1.00 | 19.76 | 8 | O |
| ATOM | 1526 | CB | ARG | A | 197 | −0.287 | 31.662 | 4.129 | 1.00 | 22.07 | 6 | C |
| ATOM | 1527 | CG | ARG | A | 197 | −1.371 | 30.658 | 3.803 | 1.00 | 25.31 | 6 | C |
| ATOM | 1528 | CD | ARG | A | 197 | −0.905 | 29.660 | 2.747 | 1.00 | 27.94 | 6 | C |
| ATOM | 1529 | NE | ARG | A | 197 | −1.833 | 29.480 | 1.689 | 1.00 | 31.60 | 7 | N |
| ATOM | 1530 | CZ | ARG | A | 197 | −2.514 | 30.055 | 0.745 | 1.00 | 34.65 | 6 | C |
| ATOM | 1531 | NH1 | ARG | A | 197 | −2.479 | 31.348 | 0.466 | 1.00 | 36.35 | 7 | N |
| ATOM | 1532 | NH2 | ARG | A | 197 | −3.356 | 29.329 | 0.001 | 1.00 | 34.88 | 7 | N |
| ATOM | 1533 | N | MET | A | 198 | 1.506 | 33.448 | 6.528 | 1.00 | 16.76 | 7 | N |
| ATOM | 1534 | CA | MET | A | 198 | 2.423 | 34.504 | 6.888 | 1.00 | 16.33 | 6 | C |
| ATOM | 1535 | C | MET | A | 198 | 3.842 | 34.089 | 6.525 | 1.00 | 17.05 | 6 | C |
| ATOM | 1536 | O | MET | A | 198 | 4.224 | 32.945 | 6.732 | 1.00 | 17.97 | 8 | O |
| ATOM | 1537 | CB | MET | A | 198 | 2.324 | 34.858 | 8.379 | 1.00 | 15.58 | 6 | C |
| ATOM | 1538 | CG | MET | A | 198 | 0.932 | 35.176 | 8.870 | 1.00 | 15.29 | 6 | C |
| ATOM | 1539 | SD | MET | A | 198 | 0.749 | 35.860 | 10.518 | 1.00 | 13.09 | 16 | S |
| ATOM | 1540 | CE | MET | A | 198 | 2.083 | 37.044 | 10.605 | 1.00 | 14.48 | 6 | C |
| ATOM | 1541 | N | ALA | A | 199 | 4.622 | 35.030 | 6.023 | 1.00 | 17.32 | 7 | N |
| ATOM | 1542 | CA | ALA | A | 199 | 6.002 | 34.750 | 5.614 | 1.00 | 17.26 | 6 | C |
| ATOM | 1543 | C | ALA | A | 199 | 6.782 | 34.027 | 6.709 | 1.00 | 17.79 | 6 | C |
| ATOM | 1544 | O | ALA | A | 199 | 6.806 | 34.523 | 7.833 | 1.00 | 17.37 | 8 | O |
| ATOM | 1545 | CB | ALA | A | 199 | 6.676 | 36.076 | 5.299 | 1.00 | 18.27 | 6 | C |
| ATOM | 1546 | N | ARG | A | 200 | 7.421 | 32.934 | 6.336 | 1.00 | 17.07 | 7 | N |
| ATOM | 1547 | CA | ARG | A | 200 | 8.242 | 32.068 | 7.144 | 1.00 | 17.73 | 6 | C |
| ATOM | 1548 | C | ARG | A | 200 | 9.704 | 32.167 | 6.711 | 1.00 | 18.72 | 6 | C |
| ATOM | 1549 | O | ARG | A | 200 | 9.995 | 32.404 | 5.536 | 1.00 | 17.50 | 8 | O |
| ATOM | 1550 | CB | ARG | A | 200 | 7.749 | 30.626 | 6.975 | 1.00 | 16.84 | 6 | C |
| ATOM | 1551 | CG | ARG | A | 200 | 8.506 | 29.516 | 7.664 | 1.00 | 17.84 | 6 | C |
| ATOM | 1552 | CD | ARG | A | 200 | 7.740 | 28.195 | 7.652 | 1.00 | 18.67 | 6 | C |
| ATOM | 1553 | NE | ARG | A | 200 | 7.706 | 27.572 | 6.338 | 1.00 | 17.04 | 7 | N |
| ATOM | 1554 | CZ | ARG | A | 200 | 8.581 | 26.687 | 5.852 | 1.00 | 18.69 | 6 | C |
| ATOM | 1555 | NH1 | ARG | A | 200 | 9.624 | 26.250 | 6.560 | 1.00 | 17.64 | 7 | N |
| ATOM | 1556 | NH2 | ARG | A | 200 | 8.413 | 26.216 | 4.606 | 1.00 | 15.92 | 7 | N |
| ATOM | 1557 | N | ASN | A | 201 | 10.653 | 32.066 | 7.635 | 1.00 | 21.23 | 7 | N |
| ATOM | 1558 | CA | ASN | A | 201 | 12.067 | 32.240 | 7.331 | 1.00 | 23.27 | 6 | C |
| ATOM | 1559 | C | ASN | A | 201 | 12.348 | 33.603 | 6.706 | 1.00 | 23.87 | 6 | C |
| ATOM | 1560 | O | ASN | A | 201 | 13.253 | 33.730 | 5.859 | 1.00 | 23.26 | 8 | O |
| ATOM | 1561 | CB | ASN | A | 201 | 12.618 | 31.112 | 6.448 | 1.00 | 24.20 | 6 | C |
| ATOM | 1562 | CG | ASN | A | 201 | 12.646 | 29.799 | 7.216 | 1.00 | 25.89 | 6 | C |
| ATOM | 1563 | OD1 | ASN | A | 201 | 12.621 | 29.777 | 8.448 | 1.00 | 28.02 | 8 | O |
| ATOM | 1564 | ND2 | ASN | A | 201 | 12.666 | 28.692 | 6.496 | 1.00 | 25.20 | 7 | N |
| ATOM | 1565 | N | LYS | A | 202 | 11.598 | 34.627 | 7.127 | 1.00 | 22.36 | 7 | N |
| ATOM | 1566 | CA | LYS | A | 202 | 11.828 | 35.966 | 6.596 | 1.00 | 23.90 | 6 | C |
| ATOM | 1567 | C | LYS | A | 202 | 12.213 | 36.873 | 7.775 | 1.00 | 23.36 | 6 | C |
| ATOM | 1568 | O | LYS | A | 202 | 11.804 | 38.039 | 7.851 | 1.00 | 24.12 | 8 | O |
| ATOM | 1569 | CB | LYS | A | 202 | 10.680 | 36.565 | 5.809 | 1.00 | 25.00 | 6 | C |
| ATOM | 1570 | CG | LYS | A | 202 | 10.478 | 36.055 | 4.394 | 1.00 | 28.96 | 6 | C |
| ATOM | 1571 | CD | LYS | A | 202 | 11.704 | 36.279 | 3.507 | 1.00 | 31.05 | 6 | C |
| ATOM | 1572 | CE | LYS | A | 202 | 11.463 | 35.809 | 2.084 | 1.00 | 32.39 | 6 | C |
| ATOM | 1573 | NZ | LYS | A | 202 | 10.052 | 35.954 | 1.637 | 1.00 | 33.25 | 7 | N |
| ATOM | 1574 | N | GLY | A | 203 | 13.042 | 36.333 | 8.667 | 1.00 | 21.01 | 7 | N |
| ATOM | 1575 | CA | GLY | A | 203 | 13.520 | 37.074 | 9.829 | 1.00 | 21.03 | 6 | C |
| ATOM | 1576 | C | GLY | A | 203 | 12.484 | 37.199 | 10.940 | 1.00 | 20.44 | 6 | C |
| ATOM | 1577 | O | GLY | A | 203 | 12.255 | 38.296 | 11.460 | 1.00 | 18.71 | 8 | O |
| ATOM | 1578 | N | ASN | A | 204 | 11.766 | 36.114 | 11.249 | 1.00 | 19.47 | 7 | N |
| ATOM | 1579 | CA | ASN | A | 204 | 10.748 | 36.135 | 12.300 | 1.00 | 20.20 | 6 | C |
| ATOM | 1580 | C | ASN | A | 204 | 9.775 | 37.287 | 12.053 | 1.00 | 19.49 | 6 | C |
| ATOM | 1581 | O | ASN | A | 204 | 9.535 | 38.141 | 12.894 | 1.00 | 18.44 | 8 | O |
| ATOM | 1582 | CB | ASN | A | 204 | 11.378 | 36.255 | 13.689 | 1.00 | 21.13 | 6 | C |
| ATOM | 1583 | CG | ASN | A | 204 | 10.402 | 36.156 | 14.855 | 1.00 | 21.82 | 6 | C |
| ATOM | 1584 | OD1 | ASN | A | 204 | 10.581 | 36.769 | 15.915 | 1.00 | 20.98 | 8 | O |
| ATOM | 1585 | ND2 | ASN | A | 204 | 9.379 | 35.335 | 14.715 | 1.00 | 20.50 | 7 | N |
| ATOM | 1586 | N | HIS | A | 205 | 9.228 | 37.293 | 10.855 | 1.00 | 19.29 | 7 | N |
| ATOM | 1587 | CA | HIS | A | 205 | 8.305 | 38.252 | 10.325 | 1.00 | 19.00 | 6 | C |
| ATOM | 1588 | C | HIS | A | 205 | 7.096 | 38.438 | 11.236 | 1.00 | 17.54 | 6 | C |
| ATOM | 1589 | O | HIS | A | 205 | 6.429 | 37.481 | 11.637 | 1.00 | 14.95 | 8 | O |
| ATOM | 1590 | CB | HIS | A | 205 | 7.913 | 37.762 | 8.915 | 1.00 | 21.31 | 6 | C |
| ATOM | 1591 | CG | HIS | A | 205 | 6.785 | 38.554 | 8.324 | 1.00 | 25.40 | 6 | C |
| ATOM | 1592 | ND1 | HIS | A | 205 | 6.786 | 39.931 | 8.239 | 1.00 | 27.49 | 7 | N |
| ATOM | 1593 | CD2 | HIS | A | 205 | 5.602 | 38.154 | 7.801 | 1.00 | 26.82 | 6 | C |
| ATOM | 1594 | CE1 | HIS | A | 205 | 5.657 | 40.345 | 7.689 | 1.00 | 28.18 | 6 | C |
| ATOM | 1595 | NE2 | HIS | A | 205 | 4.930 | 39.275 | 7.405 | 1.00 | 28.68 | 7 | N |
| ATOM | 1596 | N | CYS | A | 206 | 6.854 | 39.661 | 11.690 | 1.00 | 15.21 | 7 | N |
| ATOM | 1597 | CA | CYS | A | 206 | 5.769 | 40.001 | 12.592 | 1.00 | 16.62 | 6 | C |
| ATOM | 1598 | C | CYS | A | 206 | 5.895 | 39.280 | 13.947 | 1.00 | 16.65 | 6 | C |
| ATOM | 1599 | O | CYS | A | 206 | 4.902 | 39.159 | 14.669 | 1.00 | 15.00 | 8 | O |
| ATOM | 1600 | CB | CYS | A | 206 | 4.367 | 39.723 | 12.043 | 1.00 | 14.49 | 6 | C |
| ATOM | 1601 | SG | CYS | A | 206 | 3.897 | 40.559 | 10.516 | 1.00 | 14.57 | 16 | S |
| ATOM | 1602 | N | GLY | A | 207 | 7.105 | 38.883 | 14.333 | 1.00 | 16.79 | 7 | N |
| ATOM | 1603 | CA | GLY | A | 207 | 7.354 | 38.202 | 15.589 | 1.00 | 16.13 | 6 | C |
| ATOM | 1604 | C | GLY | A | 207 | 6.613 | 36.868 | 15.660 | 1.00 | 17.43 | 6 | C |
| ATOM | 1605 | O | GLY | A | 207 | 6.435 | 36.354 | 16.767 | 1.00 | 16.17 | 8 | O |
| ATOM | 1606 | N | ILE | A | 208 | 6.528 | 36.132 | 14.546 | 1.00 | 16.30 | 7 | N |
| ATOM | 1607 | CA | ILE | A | 208 | 5.765 | 34.883 | 14.596 | 1.00 | 17.45 | 6 | C |
| ATOM | 1608 | C | ILE | A | 208 | 6.371 | 33.839 | 15.527 | 1.00 | 16.64 | 6 | C |
| ATOM | 1609 | O | ILE | A | 208 | 5.608 | 33.151 | 16.238 | 1.00 | 17.08 | 8 | O |
| ATOM | 1610 | CB | ILE | A | 208 | 5.509 | 34.347 | 13.172 | 1.00 | 17.22 | 6 | C |
| ATOM | 1611 | CG1 | ILE | A | 208 | 4.710 | 33.042 | 13.262 | 1.00 | 17.93 | 6 | C |
| ATOM | 1612 | CG2 | ILE | A | 208 | 6.781 | 34.076 | 12.394 | 1.00 | 16.48 | 6 | C |
| ATOM | 1613 | CD1 | ILE | A | 208 | 3.346 | 33.238 | 13.832 | 1.00 | 19.94 | 6 | C |
| ATOM | 1614 | N | ALA | A | 209 | 7.686 | 33.681 | 15.579 | 1.00 | 13.80 | 7 | N |
| ATOM | 1615 | CA | ALA | A | 209 | 8.334 | 32.739 | 16.478 | 1.00 | 15.58 | 6 | C |
| ATOM | 1616 | C^{:} | ALA | A | 209 | 8.931 | 33.442 | 17.694 | 1.00 | 15.72 | 6 | C |
| ATOM | 1617 | O | ALA | A | 209 | 9.696 | 32.868 | 18.476 | 1.00 | 16.75 | 8 | O |
| ATOM | 1618 | CB | ALA | A | 209 | 9.415 | 31.916 | 15.791 | 1.00 | 15.09 | 6 | C |
| ATOM | 1619 | N | SER | A | 210 | 8.509 | 34.668 | 17.944 | 1.00 | 17.17 | 7 | N |
| ATOM | 1620 | CA | SER | A | 210 | 8.993 | 35.396 | 19.128 | 1.00 | 17.74 | 6 | C |
| ATOM | 1621 | C | SER | A | 210 | 8.594 | 34.694 | 20.420 | 1.00 | 18.87 | 6 | C |
| ATOM | 1622 | O | SER | A | 210 | 9.440 | 34.469 | 21.292 | 1.00 | 19.17 | 8 | O |
| ATOM | 1623 | CB | SER | A | 210 | 8.421 | 36.815 | 19.163 | 1.00 | 17.07 | 6 | C |
| ATOM | 1624 | OG | SER | A | 210 | 9.106 | 37.617 | 18.237 | 1.00 | 18.86 | 8 | O |
| ATOM | 1625 | N | PHE | A | 211 | 7.312 | 34.373 | 20.579 | 1.00 | 18.49 | 7 | N |
| ATOM | 1626 | CA | PHE | A | 211 | 6.816 | 33.808 | 21.830 | 1.00 | 19.86 | 6 | C |
| ATOM | 1627 | C | PHE | A | 211 | 5.921 | 32.601 | 21.639 | 1.00 | 20.65 | 6 | C |
| ATOM | 1628 | O | PHE | A | 211 | 4.708 | 32.656 | 21.886 | 1.00 | 21.46 | 8 | O |
| ATOM | 1629 | CB | PHE | A | 211 | 6.040 | 34.882 | 22.638 | 1.00 | 21.37 | 6 | C |
| ATOM | 1630 | CG | PHE | A | 211 | 6.931 | 35.904 | 23.303 | 1.00 | 22.48 | 6 | C |
| ATOM | 1631 | CD1 | PHE | A | 211 | 7.715 | 35.575 | 24.395 | 1.00 | 21.99 | 6 | C |
| ATOM | 1632 | CD2 | PHE | A | 211 | 6.998 | 37.200 | 22.815 | 1.00 | 20.63 | 6 | C |
| ATOM | 1633 | CE1 | PHE | A | 211 | 8.519 | 36.519 | 25.009 | 1.00 | 21.67 | 6 | C |
| ATOM | 1634 | CE2 | PHE | A | 211 | 7.808 | 38.143 | 23.415 | 1.00 | 22.05 | 6 | C |
| ATOM | 1635 | CZ | PHE | A | 211 | 8.604 | 37.793 | 24.495 | 1.00 | 21.72 | 6 | C |
| ATOM | 1636 | N | PRO | A | 212 | 6.465 | 31.477 | 21.195 | 1.00 | 20.25 | 7 | N |
| ATOM | 1637 | CA | PRO | A | 212 | 5.724 | 30.266 | 20.917 | 1.00 | 19.53 | 6 | C |
| ATOM | 1638 | C | PRO | A | 212 | 5.624 | 29.275 | 22.066 | 1.00 | 19.38 | 6 | C |
| ATOM | 1639 | o | PRO | A | 212 | 6.577 | 29.099 | 22.821 | 1.00 | 18.42 | 8 | O |
| ATOM | 1640 | CB | PRO | A | 212 | 6.416 | 29.641 | 19.713 | 1.00 | 20.06 | 6 | C |
| ATOM | 1641 | CG | PRO | A | 212 | 7.675 | 30.431 | 19.506 | 1.00 | 20.72 | 6 | C |
| ATOM | 1642 | CD | PRO | A | 212 | 7.869 | 31.270 | 20.756 | 1.00 | 20.14 | 6 | C |
| ATOM | 1643 | N | SER | A | 213 | 4.430 | 28.664 | 22.212 | 1.00 | 18.53 | 7 | N |
| ATOM | 1644 | CA | SER | A | 213 | 4.278 | 27.636 | 23.233 | 1.00 | 19.58 | 6 | C |
| ATOM | 1645 | C | SER | A | 213 | 3.214 | 26.607 | 22.853 | 1.00 | 20.54 | 6 | C |
| ATOM | 1646 | O | SER | A | 213 | 2.270 | 26.913 | 22.129 | 1.00 | 19.38 | 8 | O |
| ATOM | 1647 | CB | SER | A | 213 | 3.891 | 28.202 | 24.596 | 1.00 | 18.99 | 6 | C |
| ATOM | 1648 | OG | SER | A | 213 | 2.686 | 28.962 | 24.497 | 1.00 | 18.87 | 8 | O |
| ATOM | 1649 | N | TYR | A | 214 | 3.359 | 25.405 | 23.407 | 1.00 | 20.02 | 7 | N |
| ATOM | 1650 | CA | TYR | A | 214 | 2.357 | 24.372 | 23.216 | 1.00 | 22.71 | 6 | C |
| ATOM | 1651 | C | TYR | A | 214 | 2.175 | 23.497 | 24.449 | 1.00 | 23.11 | 6 | C |
| ATOM | 1652 | O | TYR | A | 214 | 3.110 | 23.177 | 25.187 | 1.00 | 21.86 | 8 | O |
| ATOM | 1653 | CB | TYR | A | 214 | 2.707 | 23.551 | 21.969 | 1.00 | 24.35 | 6 | C |
| ATOM | 1654 | CG | TYR | A | 214 | 3.959 | 22.763 | 22.261 | 1.00 | 26.72 | 6 | C |
| ATOM | 1655 | CD1 | TYR | A | 214 | 5.208 | 23.354 | 22.081 | 1.00 | 27.85 | 6 | C |
| ATOM | 1656 | CD2 | TYR | A | 214 | 3.903 | 21.473 | 22.753 | 1.00 | 27.40 | 6 | C |
| ATOM | 1657 | CE1 | TYR | A | 214 | 6.353 | 22.649 | 22.370 | 1.00 | 28.41 | 6 | C |
| ATOM | 1658 | CE2 | TYR | A | 214 | 5.038 | 20.765 | 23.051 | 1.00 | 29.19 | 6 | C |
| ATOM | 1659 | CZ | TYR | A | 214 | 6.277 | 21.359 | 22.849 | 1.00 | 29.82 | 6 | C |
| ATOM | 1660 | OH | TYR | A | 214 | 7.435 | 20.677 | 23.128 | 1.00 | 31.03 | 8 | O |
| ATOM | 1661 | N | PRO | A | 215 | 0.947 | 23.062 | 24.673 | 1.00 | 23.92 | 7 | N |
| ATOM | 1662 | CA | PRO | A | 215 | 0.598 | 22.260 | 25.840 | 1.00 | 24.95 | 6 | C |
| ATOM | 1663 | C | PRO | A | 215 | 0.759 | 20.781 | 25.551 | 1.00 | 26.05 | 6 | C |
| ATOM | 1664 | O | PRO | A | 215 | 0.854 | 20.403 | 24.384 | 1.00 | 25.20 | 8 | O |
| ATOM | 1665 | CB | PRO | A | 215 | −0.876 | 22.591 | 26.044 | 1.00 | 24.66 | 6 | C |
| ATOM | 1666 | CG | PRO | A | 215 | −1.387 | 22.722 | 24.636 | 1.00 | 23.78 | 6 | C |
| ATOM | 1667 | CD | PRO | A | 215 | −0.259 | 23.357 | 23.867 | 1.00 | 24.00 | 6 | C |
| ATOM | 1668 | N | GLU | A | 216 | 0.832 | 19.995 | 26.611 | 1.00 | 28.68 | 7 | N |
| ATOM | 1669 | CA | GLU | A | 216 | 0.848 | 18.544 | 26.494 | 1.00 | 31.39 | 6 | C |
| ATOM | 1670 | C | GLU | A | 216 | −0.057 | 17.903 | 27.541 | 1.00 | 30.14 | 6 | C |
| ATOM | 1671 | O | GLU | A | 216 | −0.172 | 18.312 | 28.694 | 1.00 | 31.42 | 8 | O |
| ATOM | 1672 | CB | GLU | A | 216 | 2.259 | 17.965 | 26.543 | 1.00 | 33.58 | 6 | C |
| ATOM | 1673 | CG | GLU | A | 216 | 2.879 | 17.836 | 25.149 | 1.00 | 36.70 | 6 | C |
| ATOM | 1674 | CD | GLU | A | 216 | 4.086 | 16.921 | 25.209 | 1.00 | 39.41 | 6 | C |
| ATOM | 1675 | OE1 | GLU | A | 216 | 4.056 | 15.930 | 25.969 | 1.00 | 41.23 | 8 | O |
| ATOM | 1676 | OE2 | GLU | A | 216 | 5.064 | 17.209 | 24.501 | 1.00 | 41.73 | 8 | O |
| ATOM | 1677 | N | ILE | A | 217 | −0.679 | 16.795 | 27.170 | 1.00 | 30.29 | 7 | N |
| ATOM | 1678 | CA | ILE | A | 217 | −1.563 | 16.044 | 28.050 | 1.00 | 29.90 | 6 | C |
| ATOM | 1679 | C | ILE | A | 217 | −0.981 | 14.698 | 28.483 | 1.00 | 31.17 | 6 | C |
| ATOM | 1680 | O | ILE | A | 217 | −0.520 | 14.629 | 29.644 | 1.00 | 32.16 | 8 | O |
| ATOM | 1681 | CB | ILE | A | 217 | −2.910 | 15.785 | 27.350 | 1.00 | 28.86 | 6 | C |
| ATOM | 1682 | CG1 | ILE | A | 217 | −3.547 | 17.119 | 26.955 | 1.00 | 27.72 | 6 | C |
| ATOM | 1683 | CG2 | ILE | A | 217 | −3.852 | 14.953 | 28.209 | 1.00 | 28.09 | 6 | C |
| ATOM | 1684 | CD1 | ILE | A | 217 | −4.742 | 17.002 | 26.044 | 1.00 | 26.56 | 6 | C |
| ATOM | 1685 | OW0 | WAT | W | 1 | −0.910 | 21.471 | 19.936 | 1.00 | 12.44 | 8 | O |
| ATOM | 1686 | OW0 | WAT | W | 2 | 10.802 | 27.216 | 8.990 | 1.00 | 19.22 | 8 | O |
| ATOM | 1687 | OW0 | WAT | W | 3 | −10.377 | 30.344 | 15.881 | 1.00 | 15.32 | 8 | O |
| ATOM | 1688 | OW0 | WAT | W | 4 | −8.315 | 24.228 | 16.799 | 1.00 | 13.99 | 8 | O |
| ATOM | 1689 | OW0 | WAT | W | 5 | −10.290 | 34.128 | 11.523 | 1.00 | 19.93 | 8 | O |
| ATOM | 1690 | OW0 | WAT | W | 6 | 9.457 | 34.855 | 8.945 | 1.00 | 15.29 | 8 | O |
| ATOM | 1691 | OW0 | WAT | W | 7 | −6.530 | 33.715 | 17.567 | 1.00 | 15.58 | 8 | O |
| ATOM | 1692 | OW0 | WAT | W | 8 | 5.156 | 34.303 | 18.682 | 1.00 | 15.74 | 8 | O |
| ATOM | 1693 | OW0 | WAT | W | 9 | −13.026 | 27.960 | 21.503 | 1.00 | 18.03 | 8 | O |
| ATOM | 1694 | OW0 | WAT | W | 10 | −1.621 | 12.831 | 31.246 | 1.00 | 32.90 | 8 | O |
| ATOM | 1695 | OW0 | WAT | W | 11 | −6.079 | 23.010 | 16.980 | 1.00 | 23.03 | 8 | O |
| ATOM | 1696 | OW0 | WAT | W | 12 | −7.127 | 39.994 | 21.014 | 1.00 | 23.94 | 8 | O |
| ATOM | 1697 | OW0 | WAT | W | 13 | 0.336 | 43.084 | 10.717 | 1.00 | 23.78 | 8 | O |
| ATOM | 1698 | OW0 | WAT | W | 14 | −12.051 | 24.811 | 5.051 | 1.00 | 17.60 | 8 | O |
| ATOM | 1699 | OW0 | WAT | W | 15 | −10.134 | 18.120 | 20.608 | 1.00 | 13.60 | 8 | O |
| ATOM | 1700 | OW0 | WAT | W | 16 | 3.564 | 31.489 | 23.974 | 1.00 | 16.67 | 8 | O |
| ATOM | 1701 | OW0 | WAT | W | 17 | 1.919 | 33.122 | 25.543 | 1.00 | 19.17 | 8 | O |
| ATOM | 1702 | OW0 | WAT | W | 18 | −17.523 | 32.721 | 10.869 | 1.00 | 18.30 | 8 | O |
| ATOM | 1703 | OW0 | WAT | W | 19 | −8.645 | 36.781 | 31.864 | 1.00 | 17.00 | 8 | O |
| ATOM | 1704 | OW0 | WAT | W | 20 | −18.368 | 41.504 | 19.911 | 1.00 | 21.43 | 8 | O |
| ATOM | 1705 | OW0 | WAT | W | 21 | 1.374 | 43.515 | 23.204 | 1.00 | 17.28 | 8 | O |
| ATOM | 1706 | OW0 | WAT | W | 22 | −6.583 | 31.141 | 16.679 | 1.00 | 23.32 | 8 | O |
| ATOM | 1707 | OW0 | WAT | W | 23 | −4.936 | 43.220 | 1.608 | 1.00 | 19.07 | 8 | O |
| ATOM | 1708 | OW0 | WAT | W | 24 | −9.086 | 19.655 | 11.727 | 1.00 | 22.26 | 8 | O |
| ATOM | 1709 | OW0 | WAT | W | 25 | −9.747 | 28.261 | 17.677 | 1.00 | 14.07 | 8 | O |
| ATOM | 1710 | OW0 | WAT | W | 26 | −10.474 | 9.886 | 15.479 | 1.00 | 31.27 | 8 | O |
| ATOM | 1711 | OW0 | WAT | W | 27 | 5.019 | 35.857 | 9.836 | 1.00 | 18.07 | 8 | O |
| ATOM | 1712 | OW0 | WAT | W | 28 | −0.417 | 15.928 | 24.512 | 1.00 | 25.06 | 8 | O |
| ATOM | 1713 | OW0 | WAT | W | 29 | 14.152 | 33.685 | 11.654 | 1.00 | 26.61 | 8 | O |
| ATOM | 1714 | OW0 | WAT | W | 30 | −0.445 | 47.894 | 6.519 | 1.00 | 23.12 | 8 | O |
| ATOM | 1715 | OW0 | WAT | W | 31 | −13.212 | 21.438 | 12.066 | 1.00 | 25.71 | 8 | O |
| ATOM | 1716 | OW0 | WAT | W | 32 | 8.859 | 43.255 | 8.773 | 1.00 | 28.91 | 8 | O |
| ATOM | 1717 | OW0 | WAT | W | 33 | −8.078 | 28.138 | 4.697 | 1.00 | 27.00 | 8 | O |
| ATOM | 1718 | OW0 | WAT | W | 34 | −9.327 | 39.744 | 5.401 | 1.00 | 24.36 | 8 | O |
| ATOM | 1719 | OW0 | WAT | W | 35 | −15.238 | 29.329 | 5.938 | 1.00 | 37.48 | 8 | O |
| ATOM | 1720 | OW0 | WAT | W | 36 | −15.682 | 36.496 | 9.919 | 1.00 | 32.49 | 8 | O |
| ATOM | 1721 | OW0 | WAT | W | 37 | −20.329 | 31.853 | 29.441 | 1.00 | 18.84 | 8 | O |
| ATOM | 1722 | OW0 | WAT | W | 38 | −15.309 | 29.578 | 21.369 | 1.00 | 37.57 | 8 | O |
| ATOM | 1723 | OW0 | WAT | W | 39 | −20.112 | 35.889 | 21.955 | 1.00 | 25.41 | 8 | O |
| ATOM | 1724 | OW0 | WAT | W | 40 | 0.568 | 16.884 | 10.867 | 1.00 | 26.71 | 8 | O |
| ATOM | 1725 | OW0 | WAT | W | 41 | −11.276 | 27.509 | 37.922 | 1.00 | 24.48 | 8 | O |
| ATOM | 1726 | OW0 | WAT | W | 42 | 10.810 | 28.531 | 18.235 | 1.00 | 24.10 | 8 | O |
| ATOM | 1727 | OW0 | WAT | W | 43 | 4.428 | 20.883 | 26.323 | 1.00 | 29.31 | 8 | O |
| ATOM | 1728 | OW0 | WAT | W | 44 | −2.909 | 42.422 | 3.165 | 1.00 | 17.38 | 8 | O |
| ATOM | 1729 | OW0 | WAT | W | 45 | 15.034 | 33.199 | 9.073 | 1.00 | 38.01 | 8 | O |
| ATOM | 1730 | OW0 | WAT | W | 46 | 16.621 | 32.433 | 20.655 | 1.00 | 23.33 | 8 | O |
| ATOM | 1731 | OW0 | WAT | W | 47 | 14.849 | 31.179 | 12.633 | 1.00 | 28.83 | 8 | O |
| ATOM | 1732 | OW0 | WAT | W | 48 | −20.277 | 34.505 | 6.785 | 1.00 | 29.98 | 8 | O |
| ATOM | 1733 | OW0 | WAT | W | 49 | −20.371 | 36.073 | 9.026 | 1.00 | 57.53 | 8 | O |
| ATOM | 1734 | OW0 | WAT | W | 50 | 8.103 | 34.049 | 2.670 | 1.00 | 21.77 | 8 | O |
| ATOM | 1735 | OW0 | WAT | W | 51 | 12.130 | 25.137 | 5.917 | 1.00 | 34.30 | 8 | O |
| ATOM | 1736 | OW0 | WAT | W | 52 | −0.770 | 38.179 | 0.446 | 1.00 | 30.27 | 8 | O |
| ATOM | 1737 | OW0 | WAT | W | 53 | −11.606 | 27.014 | 19.422 | 1.00 | 17.83 | 8 | O |
| ATOM | 1738 | OW0 | WAT | W | 54 | 11.237 | 21.841 | 18.202 | 1.00 | 24.15 | 8 | O |
| ATOM | 1739 | OW0 | WAT | W | 55 | −10.850 | 25.169 | 10.852 | 1.00 | 27.41 | 8 | O |
| ATOM | 1740 | OW0 | WAT | W | 56 | 1.649 | 50.280 | 9.162 | 1.00 | 45.96 | 8 | O |
| ATOM | 1741 | OW0 | WAT | W | 57 | −2.818 | 22.460 | 3.361 | 1.00 | 30.19 | 8 | O |
| ATOM | 1742 | OW0 | WAT | W | 58 | −6.939 | 28.751 | 18.021 | 1.00 | 21.48 | 8 | O |
| ATOM | 1743 | OW0 | WAT | W | 59 | 6.711 | 40.295 | 21.129 | 1.00 | 25.28 | 8 | O |
| ATOM | 1744 | OW0 | WAT | W | 60 | 9.491 | 23.601 | 4.589 | 1.00 | 29.21 | 8 | O |
| ATOM | 1745 | OW0 | WAT | W | 61 | 4.732 | 22.968 | 32.183 | 1.00 | 25.54 | 8 | O |
| ATOM | 1746 | OW0 | WAT | W | 62 | 3.245 | 23.888 | 0.683 | 1.00 | 27.48 | 8 | O |
| ATOM | 1747 | OW0 | WAT | W | 63 | −4.549 | 28.827 | 3.443 | 1.00 | 41.84 | 8 | O |
| ATOM | 1748 | OW0 | WAT | W | 64 | −16.762 | 21.566 | 19.087 | 1.00 | 24.93 | 8 | O |
| ATOM | 1749 | OW0 | WAT | W | 65 | −16.006 | 29.628 | 25.485 | 1.00 | 23.61 | 8 | O |
| ATOM | 1750 | OW0 | WAT | W | 66 | −19.671 | 38.994 | 19.445 | 1.00 | 21.93 | 8 | O |
| ATOM | 1751 | OW0 | WAT | W | 67 | −12.770 | 34.587 | 4.211 | 1.00 | 27.77 | 8 | O |
| ATOM | 1752 | OW0 | WAT | W | 68 | 3.613 | 28.695 | 34.171 | 1.00 | 32.54 | 8 | O |
| ATOM | 1753 | OW0 | WAT | W | 69 | 2.143 | 46.146 | 22.337 | 1.00 | 21.99 | 8 | O |
| ATOM | 1754 | OW0 | WAT | W | 70 | −11.266 | 36.608 | −0.771 | 1.00 | 43.42 | 8 | O |
| ATOM | 1755 | OW0 | WAT | W | 71 | −1.389 | 40.584 | 1.897 | 1.00 | 25.69 | 8 | O |
| ATOM | 1756 | OW0 | WAT | W | 72 | −9.590 | 34.622 | 0.707 | 1.00 | 28.56 | 8 | O |
| ATOM | 1757 | OW0 | WAT | W | 73 | −15.973 | 28.933 | 11.336 | 1.00 | 29.95 | 8 | O |
| ATOM | 1758 | OW0 | WAT | W | 74 | −11.061 | 20.499 | 37.743 | 1.00 | 39.23 | 8 | O |
| ATOM | 1759 | OW0 | WAT | W | 75 | −23.739 | 35.262 | 23.970 | 1.00 | 45.10 | 8 | O |
| ATOM | 1760 | OW0 | WAT | W | 76 | 8.589 | 41.730 | 11.384 | 1.00 | 17.86 | 8 | O |
| ATOM | 1761 | OW0 | WAT | W | 77 | −17.541 | 31.771 | 17.294 | 1.00 | 34.03 | 8 | O |
| ATOM | 1762 | OW0 | WAT | W | 78 | 9.975 | 20.417 | 21.834 | 1.00 | 41.59 | 8 | O |
| ATOM | 1763 | OW0 | WAT | W | 79 | 11.434 | 19.893 | 5.251 | 1.00 | 30.44 | 8 | O |
| ATOM | 1764 | OW0 | WAT | W | 80 | −10.671 | 43.182 | 31.401 | 1.00 | 42.36 | 8 | O |
| ATOM | 1765 | OW0 | WAT | W | 81 | −15.785 | 42.694 | 28.939 | 1.00 | 43.90 | 8 | O |
| ATOM | 1766 | OW0 | WAT | W | 82 | 13.075 | 43.095 | 17.421 | 1.00 | 28.16 | 8 | O |
| ATOM | 1767 | OW0 | WAT | W | 83 | 14.757 | 21.720 | 18.178 | 1.00 | 51.85 | 8 | O |
| ATOM | 1768 | OW0 | WAT | W | 84 | −11.832 | 32.572 | 2.024 | 1.00 | 32.71 | 8 | O |
| ATOM | 1769 | OW0 | WAT | W | 85 | 2.496 | 45.558 | 19.654 | 1.00 | 24.86 | 8 | O |
| ATOM | 1770 | OW0 | WAT | W | 86 | 3.515 | 47.175 | 15.702 | 1.00 | 43.61 | 8 | O |
| ATOM | 1771 | OW0 | WAT | W | 87 | 0.695 | 28.516 | 39.132 | 1.00 | 39.24 | 8 | O |
| ATOM | 1772 | OW0 | WAT | W | 88 | −7.830 | 26.767 | 39.676 | 1.00 | 29.78 | 8 | O |
| ATOM | 1773 | OW0 | WAT | W | 89 | −0.474 | 41.511 | 32.438 | 1.00 | 23.76 | 8 | O |
| ATOM | 1774 | OW0 | WAT | W | 90 | 2.976 | 16.622 | 6.539 | 1.00 | 29.91 | 8 | O |
| ATOM | 1775 | OW0 | WAT | W | 91 | −21.948 | 33.748 | 10.159 | 1.00 | 60.45 | 8 | O |
| ATOM | 1776 | OW0 | WAT | W | 92 | 3.371 | 21.393 | 34.375 | 1.00 | 34.65 | 8 | O |
| ATOM | 1777 | OW0 | WAT | W | 93 | −22.603 | 33.166 | 31.218 | 1.00 | 32.80 | 8 | O |
| ATOM | 1778 | OW0 | WAT | W | 94 | −7.926 | 41.009 | 17.875 | 1.00 | 36.23 | 8 | O |
| ATOM | 1779 | OW0 | WAT | W | 95 | −18.439 | 36.474 | 19.412 | 1.00 | 31.61 | 8 | O |
| ATOM | 1780 | OW0 | WAT | W | 96 | −5.429 | 41.239 | 19.675 | 1.00 | 40.52 | 8 | O |
| ATOM | 1781 | OW0 | WAT | W | 97 | −20.582 | 37.836 | 35.568 | 1.00 | 30.88 | 8 | O |
| ATOM | 1782 | OW0 | WAT | W | 98 | −23.221 | 31.607 | 29.089 | 1.00 | 34.68 | 8 | O |
| ATOM | 1783 | OW0 | WAT | W | 99 | 12.146 | 33.846 | −1.004 | 1.00 | 48.25 | 8 | O |
| ATOM | 1784 | OW0 | WAT | W | 100 | 18.822 | 24.740 | 12.299 | 1.00 | 32.46 | 8 | O |
| ATOM | 1785 | OW0 | WAT | W | 101 | 1.391 | 41.076 | 1.890 | 1.00 | 24.08 | 8 | O |
| ATOM | 1786 | OW0 | WAT | W | 102 | −10.726 | 40.370 | 33.837 | 1.00 | 33.73 | 8 | O |
| ATOM | 1787 | OW0 | WAT | W | 103 | 9.632 | 19.629 | 18.745 | 1.00 | 41.29 | 8 | O |
| ATOM | 1788 | OW0 | WAT | W | 104 | −3.386 | 16.452 | 31.884 | 1.00 | 46.20 | 8 | O |
| ATOM | 1789 | OW0 | WAT | W | 105 | −12.058 | 13.329 | 24.837 | 1.00 | 33.28 | 8 | O |
| ATOM | 1790 | OW0 | WAT | W | 106 | −7.547 | 15.008 | 28.630 | 1.00 | 35.80 | 8 | O |
| ATOM | 1791 | OW0 | WAT | W | 107 | 20.373 | 24.734 | 17.211 | 1.00 | 36.71 | 8 | O |
| ATOM | 1792 | OW0 | WAT | W | 108 | 2.610 | 31.441 | 35.560 | 1.00 | 36.40 | 8 | O |
| ATOM | 1793 | OW0 | WAT | W | 109 | 9.251 | 25.600 | 28.675 | 1.00 | 32.16 | 8 | O |
| ATOM | 1794 | OW0 | WAT | W | 110 | 11.971 | 32.083 | 3.246 | 1.00 | 30.14 | 8 | O |
| ATOM | 1795 | OW0 | WAT | W | 111 | 6.002 | 31.109 | 25.234 | 1.00 | 39.01 | 8 | O |
| ATOM | 1796 | OW0 | WAT | W | 112 | 0.433 | 24.275 | 4.740 | 1.00 | 38.25 | 8 | O |
| ATOM | 1797 | OW0 | WAT | W | 113 | −18.126 | 28.175 | 19.158 | 1.00 | 36.69 | 8 | O |
| ATOM | 1798 | OW0 | WAT | W | 114 | −12.379 | 38.438 | 32.959 | 1.00 | 36.08 | 8 | O |
| ATOM | 1799 | OW0 | WAT | W | 115 | −19.054 | 36.626 | 12.854 | 1.00 | 40.25 | 8 | O |
| ATOM | 1800 | OW0 | WAT | W | 116 | 18.602 | 30.639 | 19.399 | 1.00 | 36.96 | 8 | O |
| ATOM | 1801 | OW0 | WAT | W | 117 | 10.769 | 44.333 | 16.426 | 1.00 | 48.24 | 8 | O |
| ATOM | 1802 | OW0 | WAT | W | 118 | 10.869 | 40.767 | 8.309 | 1.00 | 34.42 | 8 | O |
| ATOM | 1803 | OW0 | WAT | W | 119 | −15.068 | 17.935 | 41.396 | 1.00 | 48.39 | 8 | O |
| ATOM | 1804 | OW0 | WAT | W | 120 | 1.692 | 15.677 | 21.014 | 1.00 | 41.42 | 8 | O |
| ATOM | 1805 | OW0 | WAT | W | 121 | 2.954 | 23.320 | 36.284 | 1.00 | 31.78 | 8 | O |
| ATOM | 1806 | OW0 | WAT | W | 122 | −19.201 | 40.341 | 36.271 | 1.00 | 49.58 | 8 | O |
| ATOM | 1807 | OW0 | WAT | W | 123 | 4.898 | 20.057 | −0.086 | 1.00 | 47.55 | 8 | O |
| ATOM | 1808 | OW0 | WAT | W | 124 | 3.752 | 14.290 | 13.359 | 1.00 | 40.38 | 8 | O |
| ATOM | 1809 | OW0 | WAT | W | 125 | −11.800 | 44.163 | 24.455 | 1.00 | 39.50 | 8 | O |
| ATOM | 1810 | OW0 | WAT | W | 126 | 11.856 | 24.824 | 24.333 | 1.00 | 36.60 | 8 | O |
| ATOM | 1811 | OW0 | WAT | W | 127 | −10.353 | 19.516 | 9.105 | 1.00 | 36.56 | 8 | O |
| ATOM | 1812 | OW0 | WAT | W | 128 | −12.367 | 15.111 | 30.129 | 1.00 | 46.42 | 8 | O |
| ATOM | 1813 | OW0 | WAT | W | 129 | −5.315 | 20.165 | 33.763 | 1.00 | 30.09 | 8 | O |
| ATOM | 1814 | OW0 | WAT | W | 130 | 11.790 | 20.099 | 11.052 | 1.00 | 45.03 | 8 | O |
| ATOM | 1815 | OW0 | WAT | W | 131 | −6.630 | 31.565 | −1.492 | 1.00 | 41.58 | 8 | O |
| ATOM | 1816 | OW0 | WAT | W | 132 | 7.326 | 21.938 | 31.335 | 1.00 | 51.41 | 8 | O |
| ATOM | 1817 | OW0 | WAT | W | 133 | 2.363 | 35.159 | 23.944 | 1.00 | 44.24 | 8 | O |
| ATOM | 1818 | OW0 | WAT | W | 134 | −6.470 | 21.515 | 4.910 | 1.00 | 29.79 | 8 | O |
| ATOM | 1819 | OW0 | WAT | W | 135 | 7.467 | 18.227 | 24.247 | 1.00 | 46.37 | 8 | O |
| ATOM | 1820 | OW0 | WAT | W | 136 | −17.299 | 17.544 | 19.488 | 1.00 | 44.37 | 8 | O |
| ATOM | 1821 | OW0 | WAT | W | 137 | −17.328 | 14.850 | 19.960 | 1.00 | 54.52 | 8 | O |
| ATOM | 1822 | OW0 | WAT | W | 138 | −2.456 | 33.205 | −3.701 | 1.00 | 42.39 | 8 | O |
| ATOM | 1823 | OW0 | WAT | W | 139 | −22.647 | 37.826 | 31.682 | 1.00 | 52.60 | 8 | O |
| ATOM | 1824 | OW0 | WAT | W | 140 | 1.944 | 34.349 | −3.049 | 1.00 | 54.44 | 8 | O |
| ATOM | 1825 | OW0 | WAT | W | 141 | −9.481 | 19.911 | 41.810 | 1.00 | 38.57 | 8 | O |
| ATOM | 1826 | OW0 | WAT | W | 142 | −16.796 | 43.302 | 25.650 | 1.00 | 37.55 | 8 | O |
| ATOM | 1827 | OW0 | WAT | W | 143 | −17.581 | 22.451 | 22.538 | 1.00 | 30.74 | 8 | O |
| ATOM | 1828 | OW0 | WAT | W | 144 | −8.342 | 9.947 | 13.611 | 1.00 | 46.58 | 8 | O |
| ATOM | 1829 | OW0 | WAT | W | 145 | 13.561 | 26.135 | 8.679 | 1.00 | 40.48 | 8 | O |
| ATOM | 1830 | OW0 | WAT | W | 146 | 11.860 | 22.971 | 2.736 | 1.00 | 47.52 | 8 | O |
| ATOM | 1831 | OW0 | WAT | W | 147 | −7.907 | 50.707 | 2.759 | 1.00 | 50.48 | 8 | O |
| ATOM | 1832 | OW0 | WAT | W | 148 | −7.759 | 7.515 | 20.288 | 1.00 | 48.12 | 8 | O |
| ATOM | 1833 | OW0 | WAT | W | 149 | 10.028 | 32.584 | 1.398 | 1.00 | 48.03 | 8 | O |
| ATOM | 1834 | OW0 | WAT | W | 150 | −3.436 | 39.000 | 24.408 | 1.00 | 31.71 | 8 | O |
| ATOM | 1835 | OW0 | WAT | W | 151 | 16.914 | 23.112 | 15.212 | 1.00 | 50.74 | 8 | O |
| ATOM | 1836 | OW0 | WAT | W | 152 | 8.418 | 49.387 | 11.663 | 1.00 | 46.17 | 8 | O |
| ATOM | 1837 | OW0 | WAT | W | 153 | −15.339 | 36.641 | 26.632 | 1.00 | 50.78 | 8 | O |
| ATOM | 1838 | OW0 | WAT | W | 154 | 8.042 | 15.079 | 11.242 | 1.00 | 49.08 | 8 | O |
| ATOM | 1839 | OW0 | WAT | W | 155 | −22.392 | 24.495 | 35.273 | 1.00 | 50.96 | 8 | O |
| ATOM | 1840 | OW0 | WAT | W | 156 | −3.356 | 19.171 | 36.223 | 1.00 | 61.39 | 8 | O |
| ATOM | 1841 | OW0 | WAT | W | 157 | 3.986 | 34.421 | 26.342 | 1.00 | 43.04 | 8 | O |
| ATOM | 1842 | OW0 | WAT | W | 158 | −7.105 | 28.768 | 0.040 | 1.00 | 44.54 | 8 | O |
| ATOM | 1843 | OW0 | WAT | W | 159 | −21.138 | 26.977 | 32.703 | 1.00 | 60.31 | 8 | O |
| ATOM | 1844 | OW0 | WAT | W | 160 | 8.193 | 16.517 | 1.801 | 1.00 | 43.37 | 8 | O |
| ATOM | 1845 | OW0 | WAT | W | 161 | −2.675 | 46.231 | 24.144 | 1.00 | 46.14 | 8 | O |
| ATOM | 1846 | OW0 | WAT | W | 162 | −20.971 | 39.299 | 16.925 | 1.00 | 48.29 | 8 | O |
| ATOM | 1847 | OW0 | WAT | W | 163 | −9.185 | 32.433 | −0.852 | 1.00 | 41.71 | 8 | O |
| ATOM | 1848 | OW0 | WAT | W | 164 | −4.405 | 11.944 | 31.263 | 1.00 | 55.39 | 8 | O |
| ATOM | 1849 | OW0 | WAT | W | 165 | −4.770 | 27.107 | −0.375 | 1.00 | 54.17 | 8 | O |
| ATOM | 1850 | OW0 | WAT | W | 166 | 1.451 | 43.839 | 2.744 | 1.00 | 49.50 | 8 | O |
| ATOM | 1851 | OW0 | WAT | W | 167 | 14.061 | 38.714 | 5.956 | 1.00 | 44.09 | 8 | O |
| ATOM | 1852 | OW0 | WAT | W | 168 | −13.353 | 37.178 | 35.666 | 1.00 | 57.16 | 8 | O |
| ATOM | 1853 | OW0 | WAT | W | 169 | 1.601 | 14.137 | 24.788 | 1.00 | 47.99 | 8 | O |
| ATOM | 1854 | OW0 | WAT | W | 170 | −14.633 | 32.421 | 2.239 | 1.00 | 49.23 | 8 | O |
| ATOM | 1855 | OW0 | WAT | W | 171 | −19.385 | 29.831 | 22.003 | 1.00 | 47.08 | 8 | O |
| ATOM | 1856 | OW0 | WAT | W | 172 | 9.097 | 19.406 | 26.244 | 1.00 | 45.97 | 8 | O |
| ATOM | 1857 | OW0 | WAT | W | 173 | 13.974 | 18.605 | 8.106 | 1.00 | 55.12 | 8 | O |
| ATOM | 1858 | OW0 | WAT | W | 174 | −14.290 | 34.894 | 39.161 | 1.00 | 55.60 | 8 | O |
| ATOM | 1859 | OW0 | WAT | W | 175 | −8.495 | 23.075 | 4.682 | 1.00 | 39.35 | 8 | O |

We claim:

1. A crystalline cathepsin S polypeptide, free of any irreversible inhibitor bound thereto.

2. A crystalline cathepsin S polypeptide comprising the amino acid sequence of any of of SEQ ID NOs: 1-8, free of any inhibitor irreversibly bound thereto.

3. A substantially pure crystalline cathepsin S polypeptide comprising the amino acid sequence of any one of SEQ ID NOs: 1-8.

4. A substantially pure crystalline cathepsin S polypeptide comprising a variant of any one of SEQ ID NOs: 1-8, which variant does not possess the activity of cathepsin B, H, K or L.

5. A cathepsin S polypeptide comprising a variant of the amino acid sequence of any one of SEQ ID NOs: 1, 2, 5 and 6, wherein the Cys25 residue is replaced with a Ser residue.

6. The cathepsin S polypeptide according to claim 4 or 5, wherein said variant comprises all or part of the cathepsin S active site.

7. The cathepsin S polypeptide according to claim 5, which comprises the amino acid sequence of any one of SEQ ID NOs: 3, 4, 7 and 8.

8. A crystalizable composition comprising a cathepsin S polypeptide which is free of any irreversible inhibitor bound thereto.

9. The crystalizable composition according to claim 8, wherein the cathepsin S polypeptide comprises a variant of the amino acid sequence of any one of SEQ ID NO: 1, 2, 5 and 6, wherein the Cys25 residue is replaced with a Ser residue.

10. The crystalizable composition according to claim 8, wherein the cathepsin S polypeptide comprises the amino acid sequence of any one of SEQ ID NO: 3, 4, 7 and 8.

11. The crystalizable composition according to claim 8, wherein the cathepsin S polypeptide is a variant of any one of SEQ ID NO:1-8, which comprises all or part of the cathepsin S active site.

12. The crystalizable composition of claim 11, wherein said active site comprises binding pockets S₁, S₂, S₃, and S₁′.

13. The crystalizable composition of claim 11, wherein said variant is a fragment.

14. The crystalizable composition of claim 13, wherein said fragment comprises at least one member of the group consisting of binding pockets S₁, S₂, S₃, and S₁′.

15. The crystalizable composition of claim 12 or 14, wherein said S₁ binding pocket comprises Gln19.

16. The crystalizable composition of claim 12 or 14, wherein said S₂ binding pocket comprises Met71, Gly137, Val138, Val162, Asn163, Gly165 and Phe211.

17. The crystalizable composition of claim 12 or 14, wherein said S₃ binding pocket comprises Gly62, Asn63, Lys64, Asn67, Gly68 and Gly69.

18. The crystalizable composition of claim 12 or 14, wherein said S₁′ binding pocket comprises Trp186.

19. The composition of claim 13, wherein said fragment is fused to another polypeptide.

20. An apparatus for producing a three-dimensional representation of:
a) a molecule or molecular complex, wherein said molecule or molecular complex comprises a binding pocket defined by structure coordinates of cathepsin S amino acids Gly68, Gly69, Phe70, Gly62, Asn63 and Lys64 according to TABLE 3; or
b) a homologue of said molecule or molecular complex, wherein said homologue comprises a binding pocket that has a root mean square deviation from the backbone atoms of said amino acids of not more than 1.5 Å, wherein said apparatus comprises:
i) an input for accessing data that includes the structure coordinates of cathepsin S amino acids Gly68, Gly69, Phe70, Gly62, Asn63 and Lys64 according to TABLE 3;
ii) a processor for processing said data into said three-dimensional representation; and
iii) a display for displaying said three-dimensional representation generated by said processor.

21. The apparatus according to claim 20, wherein said apparatus produces a three-dimensional representation of:
a) a molecule or molecular complex, wherein said molecule or molecular complex comprises a binding pocket defined by structure coordinates of cathepsin S amino acids Gly68, Gly69, Phe70, Gly62, Asn63 and Lys64 according to TABLE 3; or
b) a homologue of said molecule or molecular complex, wherein said homologue comprises a binding pocket that has a root mean square deviation from the backbone atoms of said amino acids of not more than 1.5 Å,
wherein said data includes the structure coordinates of cathepsin S amino acids Gly68, Gly69, Phe70, Gly62, Asn63 and Lys64 according to TABLE 3.

22. An apparatus for producing a three-dimensional representation of:
a) a molecule or molecular complex, wherein said molecule or molecular complex comprises a binding pocket defined by structure coordinates of cathepsin S amino acids Met71, Gly137, Val138, Val162, Gly165 and Phe211 according to TABLE 3; or
b) a homologue of said molecule or molecular complex, wherein said homologue comprises a binding pocket that has a root mean square deviation from the backbone atoms of said amino acids of not more than 1.5 Å, said apparatus comprising:
i) an input for accessing data that includes the structure coordinates of cathepsin S amino acids Met71, Gly137, Val138, Val162, Gly165 and Phe211 according to TABLE 3;
ii) a processor for processing said data into said three-dimensional representation; and
iii) a display for displaying said three-dimensional representation generated by said processor.

23. The apparatus according to claim 22, wherein said apparatus produces a three-dimensional representation of:
a) a molecule or molecular complex, wherein said molecule or molecular complex comprises a binding pocket defined by structure coordinates of cathepsin S amino acids Met71, Gly137, Val138, Val162, Gly165 and Phe211 according to TABLE 3; or
b) a homologue of said molecule or molecular complex, wherein said homologue comprises a binding pocket that has a root mean square deviation from the backbone atoms of said amino acids of not more than 1.5 Å,
wherein said data includes the structure coordinates of cathepsin S amino acids Met71, Gly137, Val138, Val162, Gly165 and Phe211 according to TABLE 3.

24. An apparatus for producing a three-dimensional representation of:
a) a molecule or molecular complex, wherein said molecule or molecular complex comprises a binding pocket defined by structure coordinates of cathepsin S amino acids Gln19, Gly23 and Cys25 according to Table 3; or
c) a homologue of said molecule or molecular complex, wherein said homologue comprises a binding pocket that has a root mean square deviation from the backbone atoms of said amino acids of not more than 1.5 Å, said apparatus comprising:
i) an input for accessing data that includes the structure coordinates of cathepsin S amino Gln19, Gly23 and Cys2 according to TABLE 3;
ii) a processor for processing said data into said three-dimensional representation; and
iii) a display for displaying said three-dimensional representation generated by said processor.

25. The apparatus according to claim 24, wherein said apparatus produces a three-dimensional representation of:
a) a molecule or molecular complex, wherein said molecule or molecular complex comprises a binding pocket defined by structure coordinates of cathepsin S amino acids Gln19, Gly23 and Cys2 according to TABLE 3; or
b) a homologue of said molecule or molecular complex, wherein said homologue comprises a binding pocket that has a root mean square deviation from the backbone atoms of said amino acids of not more than 1.5 Å,
wherein said data includes the structure coordinates of cathepsin S amino acids Gln19, Gly23 and Cys2 according to TABLE 3.

26. An apparatus for determining at least a portion of the structure coordinates corresponding to X-ray diffraction data obtained from a molecule or molecular complex, wherein said apparatus comprises an input for accessing first data that includes at least a portion of the structural coordinates of cathepsin S according to TABLE 3 and;
a) second data that includes X-ray diffraction data obtained from said molecule or molecular complex;
b) a processor for performing a Fourier transform of said first data and said second data for processing said first data and said second data into structure coordinates; and
c) a display for displaying said structure coordinates of said molecule or molecular complex.

27. The apparatus according to claim 26, wherein said molecule or molecular complex comprises a polypeptide having cathepsin S activity.

28. A method for evaluating the potential of a chemical entity to associate with:
a) a molecule or molecular complex comprising a binding pocket defined by the coordinates of cathepsin S amino acids Gly68, Gly69, Phe70, Gly62, Asn63 and Lys64 according to TABLE 3; or
b) a homologue of said molecule or molecular complex, wherein said homologue comprises a binding pocket that has a root mean square deviation from the backbone atoms of said amino acids of not more than 1.5 Å, comprising the steps of:
i) employing computational means to perform a fitting operation between the chemical entity and a binding pocket defined by structure coordinates of cathepsin S amino acids Gly68, Gly69, Phe70, Gly62, Asn63 and Lys64 according to TABLE 3, plus or minus a root mean square deviation from the backbone atoms of said amino acids of not more than 1.54 Å, and
ii) analyzing the results of said fitting operation to quantify the association between the chemical entity and the binding pocket.

29. The method according to claim 28, wherein said method evaluates the potential of a chemical entity to associate with:
a) a molecule or molecular complex comprising a binding pocket defined by the coordinates of cathepsin S amino acids Gly68, Gly69, Phe70, Gly62, Asn63 and Lys64 according to TABLE 3; or
b) a homologue of said molecule or molecular complex, wherein said homologue comprises a binding pocket that has a root mean square deviation from the backbone atoms of said amino acids of not more than 1.5 Å

30. A method for evaluating the potential of a chemical entity to associate with:
a) a molecule or molecular complex comprising a binding pocket defined by the coordinates of cathepsin S amino acids Met71, Gly137, Val138, Val162, Gly165 and Phe211 according to TABLE 3; or
b) a homologue of said molecule or molecular complex, wherein said homologue comprises a binding pocket that has a root mean square deviation from the backbone atoms of said amino acids of not more than 1.5 Å, comprising the steps of:
i) employing computational means to perform a fitting operation between the chemical entity and a binding pocket defined by structure coordinates of cathepsin S amino acids Met71, Gly137, Val138, Val162, Gly165 and Phe211 according to TABLE 3, plus or minus a root mean square deviation from the backbone atoms of said amino acids of not more than 1.5 Å, and
ii) analyzing the results of said fitting operation to quantify the association between the chemical entity and the binding pocket.

31. The method according to claim 30, wherein said method evaluates the potential of a chemical entity to associate with:
a) a molecule or molecular complex comprising a binding pocket defined by the coordinates of cathepsin S amino acids Met71, Gly137, Val138, Val162, Gly165 and Phe211 according to TABLE 3; or
b) a homologue of said molecule or molecular complex, wherein said homologue comprises a binding pocket that has a root mean square deviation from the backbone atoms of said amino acids of not more than 1.5 Å.

32. A method for evaluating the potential of a chemical entity to associate with:
a) a molecule or molecular complex comprising a binding pocket defined by the coordinates of cathepsin S amino acids Met71, Gly137, Val138, Val162, Gly165 and Phe211 according to TABLE 3; or
b) a homologue of said molecule or molecular complex, wherein said homologue comprises a binding pocket that has a root mean square deviation from the backbone atoms of said amino acids of not more than 1.5 Å, comprising the steps of:
i) employing computational means to perform a fitting operation between the chemical entity and a binding pocket defined by structure coordinates of cathepsin S amino acids Gln19, Gly23 and Cys25 according to TABLE 3, plus or minus a root mean square deviation from the backbone atoms of said amino acids of not more than 1.5 Å, and
ii) analyzing the results of said fitting operation to quantify the association between the chemical entity and the binding pocket.

33. The method according to claim 30, wherein said method evaluates the potential of a chemical entity to associate with:
a) a molecule or molecular complex comprising a binding pocket defined by the coordinates of cathepsin S amino acids Gln19, Gly23 and Cys25 according to TABLE 3; or
b) a homologue of said molecule or molecular complex, wherein said homologue comprises a binding pocket that has a root mean square deviation from the backbone atoms of said amino acids of not more than 1.5 Å.

34. The method according to any one of claims 28, 30 and 32, wherein said method evaluates the potential of a chemical entity to associate with a molecule or molecular complex:
a) defined by structure coordinates of all of the cathepsin S amino acids, as set forth in TABLE 3, or
b) a homologue of said molecule or molecular complex having a root mean square deviation from the backbone atoms of said amino acids of not more than 1.5 Å

35. A method for identifying a potential catS inhibitor molecule comprising a cathepsin S S3-like binding pocket comprising the steps of:
a) using the atomic coordinates of Gly68, Gly69, Phe70, Gly62, Asn63 and Lys64, plus or minus a root mean square deviation from the backbone atoms of said amino acids of not more than 1.5 Å, to generate a three-dimensional structure of molecule comprising a cathepsin S S3-like binding pocket;
b) employing said three-dimensional structure to design or select said potential inhibitor;
c) synthesizing said inhibitor; and
d) contacting said inhibitor with said molecule to determine the ability of said potential agonist or antagonist to interact with said molecule.

36. A method for identifying a potential catS inhibitor molecule comprising a cathepsin S S2-like binding pocket comprising the steps of:
a) using the atomic coordinates of acids Met71, Gly137, Val138, Val162, Gly165 and Phe211, plus or minus a root mean square deviation from the backbone atoms of said amino acids of not more than 1.5 Å, to generate a three-dimensional structure of molecule comprising a cathepsin S S2-like binding pocket;
b) employing said three-dimensional structure to design or select said potential inhibitor;
c) synthesizing said inhibitor; and
d) contacting said inhibitor with said molecule to determine the ability of said potential inhibitor to interact with said molecule.

37. A method for identifying a potential catS inhibitor molecule comprising a cathepsin S S1-like binding pocket comprising the steps of:
a) using the atomic coordinates of acids Gln19, Gly23 and Cys25, plus or minus a root mean square deviation from the backbone atoms of said amino acids of not more than 1.5 Å, to generate a three-dimensional structure of molecule comprising a cathepsin S S1-like binding pocket;
b) employing said three-dimensional structure to design or select said potential inhibitor;
c) synthesizing said inhibitor; and
d) contacting said inhibitor with said molecule to determine the ability of said potential inhibitor to interact with said molecule.

38. The method according to any one of claims 35-37, wherein in step (a), the atomic coordinates of all the amino acids of cathepsin S according to TABLE 3, plus or minus a root mean square deviation from the backbone atoms of said amino acids of not more than 1.5 Å are used.

39. A method for making a catS inhibitor molecule comprising a cathepsin S S2-like binding pocket comprising the steps of:
a) using the atomic coordinates of acids Met71, Gly137, Val138, Val162, Gly165 and Phe211, plus or minus a root mean square deviation from the backbone atoms of said amino acids of not more than 1.5 Å, to generate a three-dimensionsal structure of molecule comprising a cathepsin S S2-like binding pocket;
b) employing said three-dimensional structure to design or select said potential inhibitor;
c) synthesizing said inhibitor.

40. The method according to claim 39, wherein in step (a), the atomic coordinates of all the amino acids of cathepsin S according to TABLE 3, plus or minus a root mean square deviation from the backbone atoms of said amino acids of not more than 1.5 Å are used.

41. A method for producing a potential catS inhibitor molecule comprising a cathepsin S S3-like binding pocket comprising the steps of:
a) using the atomic coordinates of Gly68, Gly69, Phe70, Gly62, Asn63 and Lys64, plus or minus a root mean square deviation from the backbone atoms of said amino acids of not more than 1.5 Å, to generate a three-dimensional structure of molecule comprising a cathepsin S S3-like binding pocket;
b) employing said three-dimensional structure to design or select said potential inhibitor;
c) synthesizing said inhibitor.

42. The method according to claim 41, wherein in step (a), the atomic coordinates of all the amino acids of cathepsin S according to TABLE 3, plus or minus a root mean square deviation from the backbone atoms of said amino acids of not more than 1.5 Å are used.

43. A method for producing a potential catS inhibitor molecule comprising a cathepsin S S1-like binding pocket comprising the steps of:
a) using the atomic coordinates of Gln19, Gly23 and Cys25, plus or minus a root mean square deviation from the backbone atoms of said amino acids of not more than 1.5 Å, to generate a three-dimensional structure of molecule comprising a cathepsin S S1-like binding pocket;
b) employing said three-dimensional structure to design or select said potential inhibitor;
c) synthesizing said inhibitor.

44. The method according to claim 43, wherein in step (a), the atomic coordinates of all the amino acids of cathepsin S according to TABLE 3, plus or minus a root mean square deviation from the backbone atoms of said amino acids of not more than 1.5 Å are used.
